(19) United States
(12) Patent Application Publication
(10) Pub. No.: US 2024/0066079 A1
(43) Pub. Date: Feb. 29, 2024

(54) COMPOSITIONS COMPRISING PROPIONIBACTERIUM ACNES BACTERIOPHAGES FOR TREATING ACNE

(71) Applicant: PHI THERAPEUTICS, INC., San Francisco, CA (US)

(72) Inventors: Yug VARMA, San Francisco, CA (US); Nancy VAN PROOYEN, San Francisco, CA (US)

(73) Assignee: PHI THERAPEUTICS, INC., San Francisco, CA (US)

(21) Appl. No.: 17/819,050

(22) Filed: Aug. 11, 2022

Related U.S. Application Data
(63) Continuation of application No. 16/606,158, filed on Sep. 24, 2021.
(60) Provisional application No. 62/488,326, filed on Apr. 21, 2017.

(51) Int. Cl. A61K 35/76; A61K 9/00; A61K 31/05; A61K 31/327; A61K 31/60; A61K 33/04; A61K 35/741; A61K 38/47; A61K 45/06; A61P 17/10

(52) U.S. Cl. CPC A61K 35/76; A61K 9/0014; A61K 31/05; A61K 31/327; A61K 31/60; A61K 33/04; A61K 35/741; A61K 38/47; A61K 45/06; A61P 17/10; C12Y 302/01052

(57) ABSTRACT

Provided herein are, inter alia, compositions, systems, and methods for preventing or treating acne. Included are compositions, combinations, systems, and methods comprising at least one Propionibacterium acnes bacteriophage, at least one anti-acne compound, and a pharmaceutically acceptable carrier. Also included are compositions, combinations, and systems comprising a Propionibacterium acnes bacteriophage and an enzyme. Methods for preventing or treating acne are also provided.

### CROSS-REFERENCE TO RELATED APPLICATIONS

This application claims the benefit of priority to U.S. Provisional Application No. 62/488,326, filed Apr. 21, 2017, which is hereby incorporated by reference in its entirety and or all purposes.

### GOVERNMENT SUPPORT

This invention was made with government support under Grant No. 1R43AR068172-01 awarded by the National Institutes of Health. The government has certain rights in the invention.

### INCORPORATION-BY-REFERENCE OF SEQUENCE LISTING

The content of the text file named “052004-503001WO_SequenceListing.TXT”, which was created on Apr. 20, 2018, and is 101,782 bytes in size, is hereby incorporated by reference in its entirety.

### BACKGROUND

Acne is a nearly universal condition that affects more than 80% of all people worldwide. This chronic skin condition is complex but the main etiological agent is Propionibacterium acnes whose overgrowth leads to inflammation that causes pimples. Despite a clear need for innovation, there has not been a novel acne drug in over 30 years. Current treatments including benzoyl peroxide and antibiotics are quite ineffective, and the most effective treatment—isotretinoin—is limited to a small set of patients due to dangerous side effects (including birth defects, liver damage, and suicide).

New methods and compositions for treating for acne are needed.

### BRIEF SUMMARY

Provided herein are, inter alia, compositions, combinations, systems, and methods for preventing or treating acne.

In an aspect, provided herein is a composition comprising, consisting essentially of, or consisting of at least one Propionibacterium acnes bacteriophage, at least one anti-acne compound, and a pharmaceutically acceptable carrier.

In an aspect, provided herein is a composition that includes at least one Propionibacterium acnes bacteriophage, no more than one anti-acne compound, and a pharmaceutically acceptable carrier.

In an aspect, provided herein is a composition that includes active ingredients consisting of at least one Propionibacterium acnes bacteriophage and no more than one anti-acne compound, and a pharmaceutically acceptable carrier.

In an aspect, provided herein is a composition that includes at least one Propionibacterium acnes bacteriophage, at least one anti-acne compound, and a pharmaceutically acceptable carrier, wherein the composition does not comprise a probiotic bacterium.

In an aspect, provided herein is a composition that includes a Propionibacterium acnes bacteriophage and an enzyme.

In an aspect, provided herein is a combination comprising, consisting essentially of, or consisting of at least one Propionibacterium acnes bacteriophage and at least one anti-acne compound, wherein each of the at least one Propionibacterium acnes bacteriophage and the at least one anti-acne compound is in a composition that further includes a pharmaceutically acceptable carrier.

In an aspect, provided herein is a combination that includes a Propionibacterium acnes bacteriophage and an enzyme.

In an aspect, provided herein is a method of preventing or treating acne in a subject in need thereof, the method including administering an effective amount of a composition or combination provided herein.

### BRIEF DESCRIPTION OF THE DRAWINGS

FIG. 1. P. acnes (acne-causing, left half plate) or P. granulosum (commensal, right half plate) bacteria was plated on RCM-agar petri dishes. Sterile half-pads soaked in either minocycline or PHIT-101 (10⁷ pfu/mL) were placed on each plate. After anaerobic incubation at 37° C. for 3 days, zones of killing (arrows) appear, indicating that minocycline kills both pathogenic and commensal bacteria while PHIT-101 kills the acne-causing bacteria without disturbing commensal P. granulosum.

FIG. 2. A synthetic skin microbiome that includes P. acnes, P. granulosum, and P. avidum was grown to confluence in a test tube. It was then incubated in the presence or absence of PHIT-101 for 48 hours. The relative proportions of the three species were quantified by NGS sequencing of the 16S amplicon of the washed bacterial pellets using the Illumina MiSeq platform. PHIT-101 was able to almost completely wipe out acne-causing P. acnes, without affecting the growth of the other two commensal species.

FIG. 3. Biofilm production amongst P. acnes strains is highly variable. 96 strains of P. acnes were grown in a 96-well polystyrene microtiter plate to stimulate biofilm production, and the biofilm produced by each strain was quantified. The variability demonstrated within this set of strains demonstrates the need to quantify biofilm formation under growth conditions more similar to those found in the human pore.

FIG. 4. A screen to select enzymes that can degrade P. acnes biofilms. P. acnes was grown in polystyrene microtiter plates to stimulate biofilm production. Enzymes were added at 0.01 mg/mL to the wells and incubated at 30° C. for 30 mins. The degraded biofilm was washed away with phosphate buffered saline (PBS), and the residual biofilm in each well was quantified by staining with crystal violet and recording absorbance at 590 nm. Proteases like proteinase K and subtilisin showed good activity, and dispersin was the best glycoside depolymerase amongst those tested.

FIG. 5. Enhancement of phage with biofilm degrading enzyme (BDE) greatly increases bacterial killing. Sessile P. acnes cells were incubated with PBS (untreated), PHIT-101, or PHIT-101 and Dispersin. Cell survival was measured using the CellTiter-Blue reagent, and fluorescence was recorded at 560_{Ex}/590_{Em}. PHIT-101 was unable to kill P. acnes as effectively as in liquid culture, but addition of the biofilm degrading enzyme Dispersin greatly increased the bacterial killing to levels similar to liquid culture.

FIG. 6. Probiotic strains produce low levels of lipase in adherent culture. Probiotic P. acnes strains with known genotypes were grown under biofilm conditions in a microtiter plate. After 72 hrs of growth, the culture supernatant was filter-sterilized and incubated with 4-MU palmitate at 37 C for 4 hours to determine extracellular lipase production. The lipase production of the probiotic strains (Pr #X) was very low in comparison to pathogen, indicating a lower inflammatory potential.

FIG. 7. Probiotic strains adhere significantly less to epithelial cells than pathogenic P. acnes. Select probiotic strains were incubated with confluent A-431 epithelial cells (MOI 10). After washing the wells, cells were lifted using 0.1% Tween 80 solution and plated on BHI plates. After anaerobic incubation for 72 hours, colonies were counted. The data show that probiotic strains showed significantly lower binding to epithelial cells (* p<0.05, ** p<0.005).

FIGS. 8A-8D. Lower inflammatory potential of probiotic strain in mouse ear inflammation model. CBA/J mice (5 mice per cohort) were injected with P. acnes strains, and cytokine analysis was performed at day 5. The probiotic strain Pr #C showed significantly lower levels (* p<0.05, ** p<0.01, *** p<0.0001) of inflammatory cytokines IL-1β (FIG. 8A), IL-6 (FIG. 8B), IL-17 (FIG. 8C), and TNFα (FIG. 8D) than the pathogenic strain. Pr-C has the ProII 16S sequence.

FIG. 9. P. acnes strains have different lipase profiles in planktonic and sessile cultures. A set of two pathogenic (Path-1, Path-2) and two probiotic (Pr-1 to Pr-6) P. acnes strains were evaluated for lipase production in planktonic (gray bars) and sessile (black bars) cultures. While the lipase production of probiotic strains was not significantly different from the pathogenic strains in liquid (planktonic) culture, their lipase output in adherent culture was consistently lower than corresponding pathogenic cultures. Interestingly, variability in lipase production amongst probiotic strains was observed. The strains with lowest lipase activity were selected.

FIG. 10. illustrates life-cycles of exemplary bacteriophages. Anticlockwise from bottom left: A phage particle recognizes and adsorbs onto the surface of the host bacterium. The phage genome is injected into the bacteria. In the lysogenic life cycle, this DNA gets integrated into the bacterial genome and replicates with it for several cycles. In the lytic life cycle, the genome does not integrate and proceeds to hijack the host machinery to replicate its genome and phage structural components. The fully assembled phage then lyses the cell, typically by producing endolysins and holins at the late stage of infection. The liberated phages are now free to seek out and infect a new host bacterium, initiating another lytic cycle.

FIG. 11 illustrates the formation of exemplary bacterial biofilms. Bacterial cells land and adhere to a surface with favorable conditions for growth. They replicate to form a colony, until a certain threshold of cell density (quorum) triggers biofilm formation. The biofilm includes a mixture of polysaccharides, proteins, DNA and lipids in varying proportions. The biofilm is a physical barrier that protects the bacterial colony from harsh external conditions and grants resistance to antibiotics, toxins and immune cells.

FIG. 12 illustrates an embodiment of three components act in concert; their effects are described sequentially for exposition. An inflamed comedone is typically clogged with the biofilm produced by overgrown P. acnes (A), along with commensal skin bacteria (B). The biofilm-degrading enzyme (bolts) breaks down the P. acnes biofilm to provide better access for the other components. The bacteriophage (hexagons) then edits or specifically kills the pathogenic P. acnes and clears the infection. Finally, the probiotic bacteria (C) colonize the pore and occupy the niche of the pathogen, preventing it from growing back and recalibrating the microbiome to a healthy state.

FIG. 13 is a cartoon of a non-limiting probiotic bacterium screening process.

FIG. 14 is a graph showing that the pathogenic strain produces significantly higher ear inflammation than PBS control, while the lead probiotic strain Pr-C induces ear inflammation not significantly different from PBS control.

FIG. 15 is a graph showing that a phage remains stable in the presence of low (0.5% w/v) and high (2% w/v) concentrations of salicylic acid.

FIG. 16 is a graph showing that a phage loses its viability in the presence of benzoyl peroxide over 60 days. The rate of loss of phage viability is greater at the higher concentration (10% w/v) compared to the lower concentration (2.5% w/v).

### DETAILED DESCRIPTION

Provided herein, are, inter alia, compositions, combinations, methods, and systems for treating and preventing acne.

Salicylic acid and benzoyl peroxide are the most commonly used anti-acne agents in over-the-counter (OTC) products. The stability of phages in combination with these anti-acne agents is unknown, especially since phages diverge widely in their stability and response to external physical and chemical factors. The redox properties of benzoyl peroxide and sulfur can potentially cause the degradation of the protein coat of the phage. Previous studies have shown that exposure to peroxide increases the rate of protein degradation by destabilizing the protein and increasing its susceptibility to proteolysis (Fligiel et al. Protein degradation following treatment with hydrogen peroxide. Am J Pathol 1984, 115 (3), 418-25; Kocha et al. Hydrogen peroxide-mediated degradation of protein: different oxidation modes of copper- and iron-dependent hydroxyl radicals on the degradation of albumin. Biochim Biophys Acta 1997, 1337 (2), 319-26). Salicylic acid is noted for its protein-binding ability (Lee et al. Protein binding of acetylsalicylic acid and salicylic acid in porcine and human serum. Vet Hum Toxicol 1995, 37 (3), 224-5; Verbeeck and Cardinal, Plasma protein binding of salicylic acid, phenytoin, chlorpromazine, propranolol and pethidine using equilibrium dialysis and ultracentrifugation. Arzneimittelforschung 1985, 35 (6), 903-6), and a high affinity for the protein coat of the capsid or the tail fibers would render the phage unviable.

Surprisingly, a Propionibacterium acnes bacteriophage was found to be stable in compositions that include salicylic acid. See, for example, FIG. 15. Thus, salicylic acid is shown to be well tolerated by the phage and is a suitable anti-acne agent for co-formulation. In embodiments, the anti-keratolytic activity of the salicylic acid complements phage activity by enabling deeper penetration of the phage, thereby increasing its killing efficiency. In embodiments, phages as described herein may be combined with salicylic acid in compositions for preventing and treating acne.

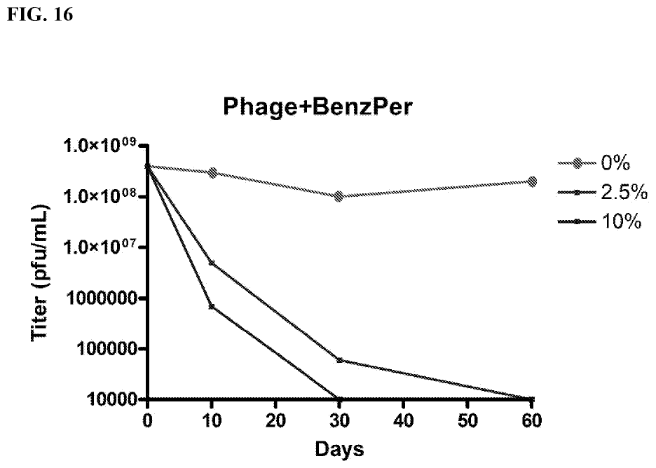

While benzoyl peroxide is not suitable for co-formulation with the phage tested (see FIG. 16) for formulations that will be stored for more than, e.g., a few days, benzoyl peroxide can be used along with a phage product as part of an anti-acne combination (e.g., a kit). In embodiments, the benzoyl peroxide is an active ingredient in a cleanser, which is applied to the skin and washed off prior to the application of a comprising the phage composition/formulation. In embodiments, the anti-keratolytic and transient antibacterial action of the benzoyl peroxide complements the specific deeper and targeted killing of P. acnes by the bacteriophage.

In embodiments, a Propionibacterium acnes bacteriophage and an anti-acne compound (such as salicylic acid and/or sulfur) are in a single composition that is topically administered to the skin of a subject. In embodiments, a kit that includes a Propionibacterium acnes bacteriophage and an anti-acne compound (e.g. in separate containers, such as bottles) is provided. In embodiments, a Propionibacterium acnes bacteriophage is in one composition and an anti-acne compound (such as benzoyl peroxide, salicylic acid, and/or sulfur) is in another composition, and each composition is topically administered to the skin of a subject. In embodiments, the Propionibacterium acnes bacteriophage is administered to the subject, and then the anti-acne compound is administered to the subject. In embodiments, the anti-acne compound is administered to the subject, and then the Propionibacterium acnes bacteriophage is administered to the subject. In embodiments, the subject's face is washed between when the anti-acne compound and the Propionibacterium acnes bacteriophage (in either order) are topically administered to the face of the subject.

In embodiments, the effective dose of the anti-acne compound (such as benzoyl peroxide, salicylic acid, or sulfur) when used in combination with the Propionibacterium acnes bacteriophage is less than would be required if the anti-acne compound was used alone. In embodiments, the effective dose of the anti-acne compound (such as benzoyl peroxide, salicylic acid, or sulfur) when used in combination with the Propionibacterium acnes bacteriophage is less than 90%, 80%, 70%, 60%, 50%, 40%, 30%, 20%, or 10% less than the dose that would be required if the anti-acne compound was used alone.

### Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure

As used herein a “Propionibacterium acnes bacteriophage” is a bacteriophage that infects, replicates within, and kills P. acnes cells. In embodiments, a P. acnes bacteriophage is a lytic P. acnes bacteriophage. In embodiments, a P. acnes bacteriophage is capable of lysing a P. acnes bacterium and incapable of lysing any bacterium which is not P. acnes. In embodiments, a P. acnes bacteriophage is incapable of sustaining lysogeny in a bacterium. In embodiments, the use of a bacteriophage that can lyse P. acnes but is incapable of sustaining lysogeny has the advantage that the bacteriophage cannot lie dormant within a bacterium, but must lyse the bacterium and hence kill it. In embodiments, a P. acnes bacteriophage lacks the ability to express at least one gene necessary for sustaining lysogeny. The term “lacks the ability to express at least one gene necessary for sustaining lysogeny” is intended to indicate that the P. acnes bacteriophage lacks the ability to produce a fully functional protein product necessary to sustain lysogeny, for example, as the result of one or more point mutations or full or partial deletions of the genome. In embodiments, the P. acnes bacteriophage has a genome that lacks all or part of at least one gene necessary for sustaining lysogeny (e.g., artificially or naturally, e.g., the strain is or is derived from a strain that lacks all or part of at least one gene necessary for sustaining lysogeny). In embodiments, the P. acnes bacteriophage may comprise defects (e.g. mutations, insertions or deletions) in the genome in non-coding regions that may, nonetheless, affect the ability of the phage to sustain lysogeny, for example defects in the genome integration site(s) (e.g. a /att/ site) or in a repressor binding site. In embodiments, a P. acnes bacteriophage is naturally occurring and isolated, with the added advantage that artificial mutations need not be introduced into the bacteriophage. In embodiments, a P. acnes bacteriophage is capable of lysing a plurality of strains of the P. acnes bacterium. In embodiments, a P. acnes bacteriophage is capable of lysing at least about 5, 10, 15, 20, 25, 30 or more strains of the P. acnes bacterium. Non-limiting examples of P. acnes bacteriophages are disclosed herein. In embodiments, the P. acnes bacteriophage has a genome having sequence identity of at least about 80%, 81%, 82%, 83%, 84%, 85%, 86%, 87%, 88%, 89%, 90%, 95%, 99%, 99.1%, 99.2%, 99.3%, 99.4%, 99.5%, 99.6%, 99.7%, 99.8%, 99.9% with SEQ ID NO: 1. In embodiments, a P. acnes bacteriophage has a genome having the sequence of SEQ ID NO: 1, or includes the sequence of SEQ ID NO: 1. In embodiments, the genome of the P. acnes bacteriophage has no insertions or deletions compared to SEQ ID NO: 1. In embodiments, the genome of the P. acnes bacteriophage has no insertions or deletions, and only conservative substitutions compared to SEQ ID NO: 1. In embodiments, the P. acnes bacteriophage is one of the following exemplary isolates of P. acnes bacteriophages that have been deposited under the terms of the Budapest Treaty at The National Collection of Industrial, Marine and Food Bacteria (NCIMB), Ferguson Building, Craibstone Estate, Bucksbum, Aberdeen, AB21 9YA, United Kingdom, under the following accession numbers: Accession no. NCIMB 41332 (isolate PA6); Accession no. NCIMB 41334 (isolate 1874); Accession no. NCIMB 41333 (isolate 1878); Accession no. NCIMB 41335 (isolate 1905); Accession no. NCIMB 41349 (isolate 1894); Accession no. NCIMB 41350 (isolate 103609); Accession no. NCIMB 41351 (isolate 103672). In embodiments, a non-limiting example of a host bacterium, P. acnes, AT1 has been deposited as NCIMB 41336. In embodiments, a P. acnes bacteriophage has a genome having sequence identity of at least about 80%, 81%, 82%, 83%, 84%, 85%, 86%, 87%, 88%, 89%, 90%, 95%, or 99% with the genome of the bacteriophage deposited under Accession No. NCIMB 41349. In embodiments, a P. acnes bacteriophage has a genome having sequence identity of at least 87% with the genome of the bacteriophage deposited under Accession No. NCIMB 41350. In embodiments, a P. acnes bacteriophage has a genome having sequence identity of at least 88% with the genome of the bacteriophage deposited under Accession No. NCIMB 41351. Additional non-limiting descriptions relating to P. acnes bacteriophages are provided in U.S. Pat. No. 9,068,159 B2, issued Jun. 30, 2015, the entire content of which is incorporated herein by reference. The terms “phage” and “bacteriophage” are used interchangeably herein.

As used herein, “degrading” a biofilm means cleaving a covalent bond of at least one compound that forms part of a biofilm (e.g., by enzymatic activity). Non-limiting examples of compounds that may form a part of a biofilm include polymers, glycosides, proteins, polysaccharides, and nucleic acids. As used herein, a “P. acnes biofilm degrading enzyme” is an enzyme that degrades at least one compound that forms part of a P. acnes biofilm.

The enzymes as provided herein include any naturally occurring forms, homologs, isoforms or variants that maintain the enzymatic activity (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

The term “isolated,” when applied to a bacterium or bacteriophage, refers to a bacterium or bacteriophage that has been (1) separated from at least some of the components with which it was associated when initially produced (whether in nature or in an experimental setting), and/or (2) produced, prepared, purified, and/or manufactured by the hand of man, e.g. using artificial culture conditions such as (but not limited to) growing on a plate and/or in a fermenter. Isolated bacteria include those bacteria that are cultured, even if such cultures are not monocultures. In embodiments, the isolated bacteria are bacteria that are cultured as a monoculture (e.g., on a plate or in liquid culture such as in a fermenter). Isolated bacteria and bacteriophages may be separated from at least about 10%, about 20%, about 30%, about 40%, about 50%, about 60%, about 70%, about 80%, about 90%, about 95%, about 96%, about 97%, about 99% or more of the other components with which they were initially associated (e.g., by weight). In embodiments, isolated bacteria are more than about 80%, about 85%, about 90%, about 91%, about 92%, about 93%, about 94%, about 95%, about 96%, about 97%, about 98%, about 99%, or more than about 99% pure (e.g., by weight). In embodiments, isolated bacteriophages are more than about 80%, about 85%, about 90%, about 91%, about 92%, about 93%, about 94%, about 95%, about 96%, about 97%, about 98%, about 99%, or more than about 99% pure (e.g., by weight). In embodiments, a composition provided herein includes one or more isolated bacteriophages. In embodiments, a composition provided herein includes an isolated bacteriophage. In embodiments, a bacteriophage that is administered is an isolated bacteriophage. In embodiments, a composition provided herein includes one or more isolated bacteria. In embodiments, a composition provided herein includes an isolated bacterium. In embodiments, a bacterium that is administered is an isolated bacterium.

A “control” sample or value refers to a sample that serves as a reference, usually a known reference, for comparison to a test sample. For example, a test sample can be taken from a test condition, e.g., in the presence of a test compound (e.g., enzyme) or phage, and compared to samples from known conditions, e.g., in the absence of the test compound, phage, or bacterium (negative control), or in the presence of a known compound, phage, or bacterium (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life, the degradation of a biofilm or a component thereof, or bacterial cell lysis) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Polynucleotides are polymers of any length, including longer lengths, e.g., 200, 300, 500, 1000, 2000, 3000, 5000, 7000, 10000, 20000, 30000, 40000 etc. Polynucleotides and oligonucleotides will generally contain phosphodiester bonds, although in some cases, nucleic acid analogs are included that may have alternate backbones, that include, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The term “bp” and the like refer, in the usual and customary sense, to the indicated number of base pairs.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual portions or domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. In embodiments, the identify exists over a region that is about or at least about 20, 50, 100, 1000, 2500, 5000, 7500, 10000, 15000, 20000, 25000, or 30000 amino acids or nucleotides in length to about, less than about, or at least about 31000, 32000, 33000, 34000 or 35000 amino acids or nucleotides in length. Optionally, the identity exists over a region that is at least about 10 to about 100, about 20 to about 75, about 30 to about 50 amino acids or nucleotides in length. Optionally, the identity exists over a region that is at least about 50 amino acids in length, or more preferably over a region that is 100 to 500 or 1000 or more amino acids in length. Included herein are phages comprising nucleic acids (e.g., a genome or a portion thereof) having sequences that are substantially identical to any of SEQ ID NOs: 1, 11, 13, 15, 17, 19, 21, 23, 25, or 27. Non-limiting examples of phages provided herein comprise genomes having sequences that are substantially identical to SEQ ID NO: 1.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions in which a sequence may be compared to a reference sequence of the same number of contiguous positions after the two sequences are optimally aligned. In embodiments, a comparison window includes about or at least about 20, 50, 100, 1000, 2500, 5000, 7500, 10000, 15000, 20000, 25000, or 30000 to about, less than about, or at least about 31000, 32000, 33000, 34000 or 35000 contiguous positions. In embodiments, a comparison window includes about or at least about 20 to about, less than about, or at least about 31000 contiguous positions. In embodiments, a comparison window includes about or at least about 25000 to about, less than about, or at least about 31000 contiguous positions. In embodiments, a comparison window includes about or at least about 26000 to about, less than about, or at least about 31000 contiguous positions. In embodiments, a comparison window includes about or at least about 27000 to about, less than about, or at least about 31000 contiguous positions. In embodiments, a comparison window includes about or at least about 28000 to about, less than about, or at least about 31000 contiguous positions. In embodiments, a comparison window includes about or at least about 29000 to about, less than about, or at least about 31000 contiguous positions. In embodiments, a comparison window includes about or at least about 30000 to about, less than about, or at least about 31000 contiguous positions. In embodiments, a comparison includes about 20 to about 600, about 50 to about 200, or about 100 to about 150 contiguous positions. In embodiments, the comparison window is the entire length of a reference sequence, such as the sequence of a bacteriophage genome. Methods of alignment of sequences for comparison are well-known in the art. In embodiments, optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information (NCBI). In embodiments, BLAST and BLAST 2.0 are used, with the parameters described herein, to determine percent sequence identity for the nucleic acids and proteins. In embodiments, a BLAST algorithm involves first identifying high scoring sequence pairs (HSPs) by identifying short words of length W in the query sequence, which either match or satisfy some positive-valued threshold score T when aligned with a word of the same length in a database sequence. In embodiments, T is referred to as the neighborhood word score threshold (Altschul et al., supra). In embodiments, these initial neighborhood word hits act as seeds for initiating searches to find longer HSPs containing them. In embodiments, the word hits are extended in both directions along each sequence for as far as the cumulative alignment score can be increased. In embodiments, cumulative scores are calculated using, for nucleotide sequences, the parameters M (reward score for a pair of matching residues; always >0) and N (penalty score for mismatching residues; always <0). In embodiments, for amino acid sequences, a scoring matrix is used to calculate the cumulative score. In embodiments, extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. In embodiments, the BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “disease” refers to any deviation from the normal health of a mammal and includes a state when disease symptoms are present, as well as conditions in which a deviation (e.g., dysbiosis, infection, gene mutation, genetic defect, etc.) has occurred, but symptoms are not yet manifested. In embodiments, the disease is acne. In embodiments, the disease includes dermal dysbiosis. In embodiments, methods, compositions, systems, phages, and probiotic bacteria provided herein are suitable for use in a subject that is a member of the Vertebrate class, Mammalia, including, without limitation, primates (such as humans), livestock, work animals, and domestic pets (e.g., a companion animal). In embodiments, a subject is a human subject. As used herein, a “symptom” of a disease includes and clinical or laboratory manifestation associated with the disease, and is not limited to what a subject can feel or observe.

As used herein, the term “dermal dysbiosis” means a difference in the skin microbiota compared to a healthy or general population. In embodiments, the dysbiosis is on the surface of the skin, within skin (e.g., within a skin region or layer of skin cells), within a gland, and/or within a pore of the skin. In embodiments, the dysbiosis is within sweat and/or sebum. In embodiments, the skin is on the face (e.g., the forehead, one or more cheeks, the nose, or the chin of a subject). In embodiments, the skin is on the shoulders, chest, or back. In embodiments, dermal dysbiosis includes a change in microbiota commensal species diversity as compared to a healthy or general population and may include decrease of beneficial microorganisms and/or increase of pathobionts (pathogenic or potentially pathogenic microorganisms) and/or decrease of overall microbiota species diversity. Many factors can lead to dysbiosis, including hormonal changes (e.g., during adolescence), infrequent washing, cosmetic use, antibiotic use, psychological and physical stress, radiation, and dietary changes.

In embodiments, compositions are administered to a subject suffering from acne in a “therapeutically effective dose.” Amounts effective for this use may depend upon the severity of the disease and the general state of the patient's health. Single or multiple administrations of the compositions may be administered depending on the dosage and frequency as required and tolerated by the patient. A “patient” or “subject” includes both humans and other animals, particularly mammals. Thus the methods are applicable to both human therapy and veterinary applications.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the bacteriophages, probiotic bacteria, and/or compounds of the invention. One of skill in the art will recognize that other pharmaceutical excipients are useful in the present invention.

The term “contacting” may include allowing two species to react, interact, or physically touch, wherein the two species may be, for example, an enzyme as described herein and a biofilm that includes a substrate of the enzyme. In another example, the two species may be a bacteriophage and a cell of a species that the bacteriophage infects. In embodiments contacting includes, for example, allowing a bacteriophage as described herein to interact with a P. acnes cell. In embodiments contacting includes, for example, allowing an enzyme as described herein to interact with a P. acnes biofilm.

“Patient” or “subject in need thereof” refers to a living member of the animal kingdom suffering from or who may suffer from the indicated disorder. In embodiments, the subject is a member of a species that includes individuals who naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

As used herein the abbreviation “sp.” for species means at least one species (e.g., 1, 2, 3, 4, 5, or more species) of the indicated genus. The abbreviation “spp.” for species means 2 or more species (e.g., 2, 3, 4, 5, 6, 7, 8, 9, 10 or more) of the indicated genus. In embodiments, methods and compositions provided herein comprise a single species within an indicated genus or indicated genera, or 2 or more (e.g., a plurality that includes more than 2) species within an indicated genus or indicated genera. In embodiments, 1, 2, 3, 4, 5, or more or all or the indicated species is or are isolated. In embodiments, the indicated species are administered together. In embodiments, each of the indicated species is present in a single composition that includes each of the species. In embodiments, each of the species is administered concurrently, e.g., within about 1, 2, 3, 4, 5, 6, 7, 8, 9, 10, 30, or 60, 1-5, 1-10, 1-30, 1-60, or 5-15 seconds or minutes of each other.

In this disclosure, “comprises,” “comprising,” “containing,” and “having” and the like can have the meaning ascribed to them in U.S. Patent law and can mean “includes,” “including,” and the like. Thus, the transitional term “comprising,” which is synonymous with “including,” “containing,” or “characterized by,” is inclusive or open-ended and does not exclude additional, unrecited features, integers, steps, operations, elements, and/or components. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. By contrast, the transitional phrase “consisting of” excludes any feature, integer, element, step, operation, component, and/or ingredient not specified.

As used herein, the term “about” in the context of a numerical value or range means ±10% of the numerical value or range recited or claimed, unless the context requires a more limited range.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” herein and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently. In the case of treating acne, the terms can refer to reducing, e.g., dermal dysbiosis and/or the number or size of cystic lesions, whiteheads (closed plugged pores), blackheads (open plugged pores—in which oil exposed to the air has a dark color, e.g., brown or black), mall red, tender bumps (papules), pimples (pustules; papules with pus at their tips), large, solid, painful lumps beneath the surface of the skin (nodules).

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. Treatment can refer to any delay in onset, amelioration of symptoms, improvement in patient skin appearance, etc. The effect of treatment can be compared to an individual or pool of individuals not receiving the treatment, or to the same patient prior to treatment or at a different time during treatment. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. In embodiments, treatment is effective to reduce at least one symptom of acne. In embodiments, treatment is effective to reduce the level of pimples (pustules) on the face, forehead, chest, back, and/or shoulders of the subject. In embodiments, treatment is effective to reduce the level of whiteheads (closed plugged pores) on the face, forehead, chest, back, and/or shoulders of the subject. In embodiments, treatment is effective to reduce the level of blackheads (open plugged pores) on the face, forehead, chest, back, and/or shoulders of the subject. In embodiments, treatment is effective to reduce the level of papules on the face, forehead, chest, back, and/or shoulders of the subject. In embodiments, treatment is effective to reduce the level of solid, painful lumps beneath the surface of the skin (nodules) on the face, forehead, chest, back, and/or shoulders of the subject. In embodiments, treatment is effective to reduce the level of cystic lesions on the face, forehead, chest, back, and/or shoulders of the subject. In embodiments, the level (e.g., number) is reduced compared to before treatment has begun. In embodiments, the level (e.g., number) is reduced compared to a corresponding subject who is afflicted with acnes but who has not received treatment. In embodiments, the level (e.g., number) is reduced compared to a corresponding subject who is afflicted with acnes but who has not received treatment comprising a bacteriophage.

The terms “effective amount,” “effective dose,” “therapeutically effective amount,” etc. refer to the amount of an agent that is sufficient to ameliorate a disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “diagnosis” refers to a relative probability a subject has a given metabolic disorder. Symptoms and diagnostic criteria are summarized herein. Similarly, the term “prognosis” refers to a relative probability that a certain future outcome may occur in the subject. For example, in the context of the present invention, prognosis can refer to the likelihood that an individual will develop acne. Prognosis can also refer to the likely severity of the disease (e.g., severity of symptoms, rate of functional decline, etc.). The terms are not intended to be absolute, as will be appreciated by any one of skill in the field of medical diagnostics.

### Compositions and Combinations Comprising Bacteriophages

In an aspect, provided herein is a composition comprising, consisting essentially of, or consisting of at least one P. acnes bacteriophage, at least one anti-acne compound, and a pharmaceutically acceptable carrier.

In an aspect, provided herein is a composition that includes at least one Propionibacterium acnes bacteriophage, no more than one anti-acne compound, and a pharmaceutically acceptable carrier.

In an aspect, provided herein is a composition that includes active ingredients consisting of at least one Propionibacterium acnes bacteriophage and no more than one anti-acne compound, and a pharmaceutically acceptable carrier.

In an aspect, provided herein is a composition that includes at least one P. acnes bacteriophage, at least one anti-acne compound, and a pharmaceutically acceptable carrier, wherein the composition does not comprise a probiotic bacterium.

In embodiments, the at least one anti-acne compound is benzoyl peroxide. In embodiments, the benzoyl peroxide is present at a concentration of 2.5% to 10% (weight/volume). In embodiments, the benzoyl peroxide is present at a concentration of less than 2.5% but greater than about 0.1%, 0.5%, 1%, 1.5%, or 2% (weight/volume). In embodiments, the benzoyl peroxide is present at a concentration of 2.5% to 10%, e.g., about 2.5%, 3%, 3.5%, 4%, 4.5%, 5%, 5.5%, 6%, 6.5%, 7%, 7.5%, 8%, 8.5%, 9%, 9.5% or 10% (weight/volume). In embodiments, the benzoyl peroxide is present at a concentration of less than 2.5% but greater than about 0.1%, 0.5%, 1%, 1.5%, or 2% (weight/volume).

In embodiments, the at least one anti-acne compound is salicylic acid. In embodiments, the salicylic acid is present at a concentration of 0.5% to 2% (weight/volume). In embodiments, the salicylic acid is present at a concentration of less than 0.5% but greater than about 0.1% (weight/volume). In embodiments, the salicylic acid is present at a concentration of 0.5% to 2%, e.g., about 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% (weight/volume). In embodiments, the salicylic acid is present at a concentration of less than 0.5% but greater than about 0.1% (weight/volume).

In embodiments, the at least one anti-acne compound is sulfur. In embodiments, the sulfur is present at a concentration of 3% to 10% (weight/volume). In embodiments, the sulfur is present at a concentration of less than 3% but greater than about 0.1%, 0.5%, 1%, 1.5%, 2%, or 2.5% (weight/volume). In embodiments, the sulfur is present at a concentration of 3% to 10%, e.g., about 3%, 3.5%, 4%, 4.5%, 5%, 5.5%, 6%, 6.5%, 7%, 7.5%, 8%, 8.5%, 9%, 9.5%, or 10% (weight/volume). In embodiments, the sulfur is present at a concentration of less than 3% but greater than about 0.1%, 0.5%, 1%, 1.5%, 2%, or 2.5% (weight/volume). In embodiments, resorcinol is present at a concentration of 2% and sulfur is present at a concentration of 3% to 8% (e.g., about 2.5%, 3%, 3.5%, 4%, 4.5%, 5%, 5.5%, 6%, 6.5%, 7%, 7.5%, or 8%) (weight/volume).

In embodiments, the at least one anti-acne compound is resorcinol and sulfur. In embodiments, the resorcinol is present at a concentration of 2% and sulfur is present at a concentration of 3% to 8% (weight/volume). In embodiments, resorcinol is present at a concentration of 2% and sulfur is present at a concentration of 3% to 8% (e.g., about 2.5%, 3%, 3.5%, 4%, 4.5%, 5%, 5.5%, 6%, 6.5%, 7%, 7.5%, or 8%) (weight/volume).

In embodiments, the at least one anti-acne compound includes resorcinol monoacetate and sulfur. In embodiments, the resorcinol monoacetate is present at a concentration of 3% and sulfur is present at a concentration of 3% to 8% (weight/volume). In embodiments, resorcinol monoacetate is present at a concentration of 3% and sulfur is present at a concentration of 3% to 8% (e.g., about 2.5%, 3%, 3.5%, 4%, 4.5%, 5%, 5.5%, 6%, 6.5%, 7%, 7.5%, or 8%) (weight/volume).

In embodiments, the P. acnes bacteriophage is present in an amount of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ plaque forming units (pfu). In embodiments, the P. acnes bacteriophage is present in an amount of about 1×10⁶ to 1×10¹¹ pfu. In embodiments, the P. acnes bacteriophage is present in an amount of about 1×10⁶ to 1×10⁸, about 1×10⁸ to 1×10⁹, about 1×10⁹ to 1×10¹⁰, about 1×10⁹ to 1×10¹¹ or about 1×10¹⁰ to 1×10¹¹ pfu.

In embodiments, a probiotic bacterium is present in an amount of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ colony forming units (cfu). In embodiments, the probiotic bacterium is present in an amount of about 1×10⁶ to 1×10¹¹ cfu. In embodiments, the probiotic bacterium is present in an amount of about 1×10⁶ to 1×10⁸, about 1×10⁸ to 1×10⁹, about 1×10⁹ to 1×10¹⁰, about 1×10⁹ to 1×10¹¹ or about 1×10¹⁰ to 1×10¹¹ cfu.

In embodiments, the anti-acne compound is an antibiotic, a retinoid, or an alpha-hydroxy acid.

In an aspect, provided herein is a composition that includes a P. acnes bacteriophage and an enzyme.

In an aspect, provided herein is a combination comprising, consisting essentially of, or consisting of at least one P. acnes bacteriophage, at least one anti-acne compound, wherein each of the at least one P. acnes bacteriophage and the at least one anti-acne compound is in a composition that further includes a pharmaceutically acceptable carrier.

In an aspect, provided herein is a combination that includes a P. acnes bacteriophage and an enzyme.

In embodiments, the P. acnes bacteriophage has a linear double stranded DNA genome.

In embodiments, the P. acnes bacteriophage is within the bacteriophage family Siphoviridae.

In embodiments, the bacteriophage is a wild-type bacteriophage. In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the genomic sequence of a wild-type P. acnes bacteriophage. A non-limiting example of an genomic sequence for a wild-type P. acnes bacteriophage is as follows:

| (SEQ ID NO: 1) | |
|---|---|
| 1 | AGTGAAATAC CTCCCTTTTG TGGTTTTGTC TGTTTGTCGA CTTTTTGTGT TGGTGGTGAG |
| 61 | TGTTGTGCAG CCTGAGCTTC CTGAGTCTCG TGAGTGGTGT GGGGAGACGC GTCGTTGGTG |
| 121 | GCGTGTGTGG GGTGAGGATA GTCGCGCGCC GTATGTGTCT GATGAGGAGT GGTTGTTTCT |
| 181 | TATGGATGCT GCGGTGATTC ATGATTGTGT GTGGCGTGAG GGTCGCGCGG ATTTGGTGGC |
| 241 | TTCGCTTCGT GCGCATGTGA AGGCTTTTAT GGGCATGTTG GATAGGTATT CGGTTGATGT |
| 301 | GGCGTCTGGT GGCCGTGGTG GGGGTTCTGC TGTGGCGATG ATTGACCGGT ATAGGAAGCG |
| 361 | TAGGGGGGCT TGAGTAGGTG TCTGGTGTTG TTGGGTCTCA GGTTCCTCGT CACCGTGTGG |
| 421 | CTGCGGCGTA TTCGGTGTCT GCTGGGGGTG ATGCTGGGGA GCTTGGTCGT GCGTATGGGT |
| 481 | TGACGCCTGA TCCGTGGCAG CAGCAGGTGT TGGATGATTG GCTGGCTGTC GGTAGCAATG |
| 541 | GCAGGCTTGC TTCTGGTGTG TGTGGGGTGT TTGTTCCGCG GCAGAATGGC AAGAATGCTA |
| 601 | TTTTGGAGAT TGTGGAGTTG TTTAAGGCGA CTATTCAGGG TCGCCGTATT TTGCATACGG |
| 661 | CTCACGAGTT GAAGTCGGCT CGTAAGGCGT TTATGCGGTT GAGGTCGTTT TTTGAGAATG |
| 721 | AGCGGCAGTT TCCTGACTTG TATCGTATGG TGAAGTCGAT TCGTGCGACG AATGGTCAGG |
| 781 | AGGCTATTGT GTTGCATCAT CCGGATTGTG CCACTTTTGA GAAGAAGTGT GGCTGCAGCG |
| 841 | GTTGGGGTTC GGTTGAGTTT GTGGCTCGTA GCCGGGGTTC GGCTCGCGGG TTTACGGTTG |
| 901 | ATGATTTGGT GTGTGATGAG GCTCAGGAGT TGTCGGATGA GCAGTTGGAG GCTTTGCTTC |
| 961 | CTACGGTAAG TGCTGCCCCG TCTGGTGATC CGCAGCAGAT TTTCCTTGGT ACGCCGCCTG |
| 1021 | GGCCGTTGGC TGATGGTTCT GTGGTGTTGC GTTTGCGTGG GCAGGCGCTT GGTGGCGGTA |
| 1081 | AAAGGTTTGC GTGGACGGAG TTTTCGATTC CTGACGAGTC TGATCCGGAT GATGTGTCGC |
| 1141 | GGCAGTGGCG GAAGTTGGCG GGGGATACGA ATCCGGCGTT GGGGCGTCGC CTGAATTTTG |
| 1201 | GGACCGTAAG CGATGAGCAT GAGTCGATGT CTGCTGCCGG TTTTGCTCGG GAGCGGCTTG |
| 1261 | GCTGGTGGGA TCGTGGCCAG TCTGCTGCGT CTGTGGTTCC TGCTGATAAG TGGGCTCAGT |
| 1321 | CTGCGGTGGA TGAGGCGAGT CTGGTTGGCG GGAAAGTGTT TGGTGTCTCG TTTTCTCGTT |
| 1381 | CTGGGGATCG GGTTGCTTTG GCGGGTGCCG GCAAGACTGA TGCTGGGGTT CATGTTGAGG |
| 1441 | TTATTGATGG GCTGTCGGGA ACGATTGTTG ATGGTGTGGG CCGGTTGGCT GACTGGTTGG |
| 1501 | CGGTTCGTTG GGGTGATACT GACCGGATCA TGGTTGCCGG GTCTGGTGCG GTGTTGTTGC |
| 1561 | AGAAGGCGTT GACGGATCGT GGTATTCCGG GCCGTGGCGT GGTGGTTGCT GATACTGGCG |
| 1621 | TTTATGTGGA GGCTTGTCAG GCGTTTCTTG AGGGTGTCAG GTCGGGTGTG ATCAGTCATC |
| 1681 | CTCGTGCTGA TTCTCGCCGT GACATGTTGG ATATTGCTGT GAGGTCGGCT GTGCAGAAGC |
| 1741 | GTAAGGGGTC TGCGTGGGGT TGGGGTTCCT CGTTTAAGGA TGGTTCTGAG GTTCCTTTGG |
| 1801 | AGGCTGTGTC TTTGGCGTTT TTGGGGGCTA AACGTGTTCG TCGTGGCCGT CGGGAGCGTA |
| 1861 | GTGGTAGGAA GCGGGTGTCT GTGGTATGAA CTCGGATGAG TTGGCTCTGA TTGAGGGCAT |
| 1921 | GTACGATCGT ATCCAAAGGT TGTCTTCGTG GCATTGTTGT ATTGAGGGCT ACTATGAGGG |
| 1981 | CTCTAATCGG GTGCGTGACC TTGGTGTGGC TATTCCGCCG GAGTTGCAGC GTGTGCAGAC |
| 2041 | TGTGGTGTCG TGGCCTGGTA TAGCTGTGGA TGCTTTGGAG GAGCGTCTGG ATTGGCTTGG |
| 2101 | CTGGACTAAT GGTGACGGCT ACGGCCTTGA TGGTGTGTAT GCTGCGAATC GGCTTGCTAC |
| 2161 | GGCGTCGTGT GATGTGCATT TGGATGCGCT GATTTTTGGG TTGTCGTTTG TTGCGATCAT |
| 2221 | TCCTCATGGT GATGGTACGG TGTCGGTTCG TCCGCAGTCA CCAAAGAATT GTACGGGCAA |
| 2281 | GTTTTCGGCT GACGGGTCTC GTTTGGATGC GGGTTTGGTG GTGCAGCAGA CGTGTGATCC |
| 2341 | TGAGGTTGTT GAGGCTGAGC TTTTGCTTCC TGATGTGATT GTTCAGGTGG AGCGGCGGGG |
| 2401 | TTCGCGTGAA TGGGTTGAGG TGGATCGTAT ACCGAATGTG TTGGGTGCGG TTCCGTTGGT |
| 2461 | GCCTATTGTG AATCGTCGCC GTACTTCTAG GATTGATGGC CGTTCGGAGA TTACGAGGTC |
| 2521 | TATTAGGGCT TACACGGATG AGGCTGTGCG CACACTGTTG GGGCAGTCTG TGAATCGTGA |
| 2581 | TTTTTATGCG TATCCTCAGC GTTGGGTGAC TGGCGTGAGC GCGGATGAGT TTTCGCAGCC |
| 2641 | TGGCTGGGTC CTGTCGATGG CTTCTGTGTG GGCTGTGGAT AAGGATGATG ACGGTGACAC |
| 2701 | TCCGAATGTG GGGTCGTTTC CTGTCAATAG TCCTACACCG TATTCGGATC AGATGAGACT |
| 2761 | GTTGGCGCAG TTGACTGCGG GTGAGGCGGC TGTTCCGGAA CGCTATTTCG GGTTTATCAC |
| 2821 | GTCTAACCCA CCTAGTGGGG AGGCTTTGGC TGCCGAGGAA TCTCGGCTTG TGAAGCGTGC |
| 2881 | TGAGCGGCGT CAAACGTCGT TTGGTCAGGG TTGGCTGTCG GTTGGTTTTT TGGCTGCCAA |
| 2941 | GGCGTTGGAT TCTCGTGTTG ATGAGGCCGA TTTTTTTGGT GATGTTGGTT TGCGTTGGCG |
| 3001 | TGATGCTTCG ACGCCTACCC GGGCGGCTAC GGCTGATGCT GTGACGAAGC TTGTTGGTGC |
| 3061 | CGGTATTTTG CCTGCTGATT CTCGTACGGT GTTGGAGATG TTGGGGCTTG ATGATGTGCA |
| 3121 | GGTTGAGGCT GTGATGCGTC ATCGTGCTGA GTCGTCTGAC CCGTTGGCGG TGCTTGCTGG |
| 3181 | GGCTATATCG CGTCAAACTA ACGAGGTATG ATAGGCGATG GCTTCGGGGG TTGAGGCGAG |
| 3241 | GCTTGCGGCG ACTGAGTATC AGCGTGAGGC GGTCAGGTTT GCTGGGAAGT ATGCGGGCTA |
| 3301 | TTATTCTGAG CTTGGTCGTT TGTGGCGTGC CGGCAGGATG AGTGACACGC AGTATGTGCG |
| 3361 | TTTGTGTGTG GAGTTGGAGC GTGCCGGCCA TGATGGTTCG GCATCGTTGG CTGCCAGGTT |
| 3421 | TGTGTCGGAT TTTCGCCGGT TGAATGGTGT GGATCCGGGT TTGATTGTGT ATGACGAGTT |
| 3481 | TGATGCTGCG GCGGCTTTGG CTAGGTCTAT TTCGACCACG AAGATTCTTG AGAGTGACCC |
| 3541 | GGATAGGGCG AATGACACGA TTGATGCGAT GGCGGCGGGT TTTGATCGGG CTGTTATGAA |
| 3601 | TGCTGGCCGT GACACGGTTG AGTGGTCTGC GGGTGCGCAG GGTAGGTCGT GGCGTCGGGT |
| 3661 | GACGGATGGT GATCCGTGTG CTTTTTGTGC CATGTTGGCT ACGAGGTCGG ATTATACGAC |
| 3721 | AAAAGAGAGG GCACTTACTA CTGGACATAC TCGGCGTCAT AAGCGTGGTG GTAAGCGTCC |
| 3781 | GTTTGGTTCG AAGTATCATG ATCATTGTGG TTGTACGGTG GTTGAGGTTG TTGGCCCTTG |
| 3841 | GGAACCAAAT AGGGCTGATG CCGAGTATCA GAGGACGTAT GAGAAGGCCT GTGAGTGGGT |
| 3901 | TGATGATCAT GGGTTGCAGC AATCGCCTGG CAATATTTTG AAGGCTATGC GTACTGTTGG |
| 3961 | CGACATGAGA TAATTTGATG TGGTTTCCGG TTGTGCGCCG CCGGTTATTG GTGCACAGGG |
| 4021 | TTGTCTCCCG CACGGGGGTC AACAATATTG TGTTGTTTTC CGCAAGGAGT GTAGGGTTAG |
| 4081 | GCTATGGCCG ATCAGAGTGT TGAGGAACAG AATGTTGACA ATGATGTTGT GGAGTCCGGA |
| 4141 | AAGGATAACG GCATTGTTGA TACAGTAAAA GACGATGGCG GGCAGGAGGT AGCCGACAAT |
| 4201 | CAGTTGAAGA ATGAAGGCGA GGGTAAATCG CCGGGGACTG ATTGGAAGGC TGAGGCCCGT |
| 4261 | AAGTGGGAGT CTCGTGCTAA AAGTAATTTT GCCGAGTTGG AGAAGCTTCG CGCCTCGGAT |
| 4321 | GGTGATGCGG GGTCTACGAT TGATGAGCTT CGCCGCAAGA ATGAGGAACT CGAAGACCGG |
| 4381 | ATCAATGGGT TTGTTCTTGA GGGTGTGAAG CGCGAGGTGG CTGCCGAGTG TGGCCTGTCG |
| 4441 | GGTGATGCTG TCGCTTTCTT GTCGGGTGGC GATAAGGAGT CGCTTGCCGA GTCTGCGAAA |
| 4501 | GCTTTGAAGG GTTTGATCGA CCATAGTAGT GGTGGCGCGG GTGTGCGCCG TCTTGCGGGG |
| 4561 | AGTGCCCCCG TTGATGATGT TAAACGACGT GAGGGTGTCG CGTTTGTGGA TGCTCTTGTC |
| 4621 | AATAATTCTA GGAGATGATT TGTGATGGCT GACGATTTTC TTTCTGCAGG GAAGCTTGAG |
| 4681 | CTTCCTGGTT CTATGATTGG TGCGGTTCGT GACCGTGCTA TCGATTCTGG TGTTTTGGCG |
| 4741 | AAGCTTTCGC CGGAGCAGCC GACTATTTTC GGGCCTGTGA AGGGTGCCGT GTTTAGTGGT |
| 4801 | GTTCCTCGCG CCAAGATTGT TGGTGAGGGC GAGGTTAAGC CTTCCGCGTC TGTTGATGTT |
| 4861 | TCGGCGTTTA CTGCGCAGCC TATCAAGGTT GTGACTCAGC AGCGTGTCTC GGATGAGTTT |
| 4921 | ATGTGGGCTG ATGCTGATTA CCGTCTGGGT GTGCTTCAGG ATCTGATTTC CCCGGCTCTT |
| 4981 | GGTGCTTCGA TTGGTCGCGC CGTGGATCTG ATTGCTTTCC ATGGTATTGA TCCTGCCACT |
| 5041 | GGTAAAGCGG CTTCCGCTGT GCATACTTCG CTGAATAAGA CGAAGAATAT TGTTGATGCC |
| 5101 | ACGGATTCTG CTACGGCTGA TCTTGTTAAG GCTGTCGGCC TGATTGCTGG TGCTGGTTTG |
| 5161 | CAGGTTCCTA ACGGGGTTGC TTTGGATCCG GCGTTCTCGT TTGCGCTGTC TACTGAGGTG |
| 5221 | TATCCGAAGG GGTCTCCGCT TGCCGGTCAG CCTATGTATC CTGCCGCCGG GTTTGCCGGT |
| 5281 | TTGGATAATT GGCGCGGGCT GAATGTTGGT GCTTCTTCGA CTGTTTCTGG CGCCCCGGAG |
| 5341 | ATGTCGCCTG CCTCTGGCGT TAAGGCTATT GTTGGTGATT TCTCTCGTGT TCATTGGGGT |
| 5401 | TTCCAGCGTA ACTTCCCGAT CGAGCTTATC GAGTATGGTG ACCCGGATCA GACTGGGCGT |
| 5461 | GACTTGAAGG GCCATAATGA GGTTATGGTT CGTGCCGAGG CTGTCCTGTA TGTTGCGATT |
| 5521 | GAGTCGCTTG ATTCGTTTGC TGTTGTGAAG GAGAAGGCTG CCCCGAAGCC TAATCCGCCG |
| 5581 | GCCGAGAACT GATTCATTTG TTGCGGTGAT GTTTTCTATG TGCAGGGGGT GGTGTTGATG |
| 5641 | GGTATCATTT TGAAGCCTGA GGATATTGAG CCTTTCGCCG ATATTCCTAG AGAGAAGCTT |
| 5701 | GAGGCGATGA TTGCCGATGT GGAGGCTGTG GCTGTCAGTG TCGCCCCCTG TATCGCTAAA |
| 5761 | CCGGATTTCA AATACAAGGA TGCCGCTAAG GCTATTCTGC GCAGGGCCCT GTTGCGCTGG |
| 5821 | AATGATACCG GGGTTTCGGG TCAGGTGCAG TACGAGTCTG CGGGCCCGTT TGCTCAGACT |
| 5881 | ACACGGTCGA ATACTCCCAC GAATTTGTTG TGGCCTTCTG AGATTGCCGC GTTGAAGAAG |
| 5941 | TTGTGTGAGG GTGATGGTGG GGCTGGTAAA GCGTTCACTA TTACACCGAC CATGAGGAGT |
| 6001 | AGTGTGAATC ATTCTGAGGT GTGTTCCACG GTGTGGGGTG AGGGTTGCTC GTGCGGATCT |
| 6061 | GATATTAACG GCTATGCTGG CCCTTTGTGG GAGATATGAT ATGACCGGTT TTCCTTACGG |
| 6121 | TGAAACGGTT GTGATGCTTC AACCGACTGT TCGTGTCGAT GATCTTGGCG ACAAGGTGGA |
| 6181 | AGACTGGTCT AAGCCTGTCG AGACTGTGTA CCATAACGTG GCCATCTATG CTTCCGTTTC |
| 6241 | GCAGGAGGAT GAGGCTGCCG GCCGTGACTC TGACTATGAG CATTGGTCGA TGCTTTTCAA |
| 6301 | GCAGCCTGTT GTGGGTGCCG GTTATCGTTG CCGGTGGCGT ATTCGGGGTG TGGTTTGGGA |
| 6361 | GGCGGACGGG TCTCCTATCG TGTGGCATCA TCCGATGTCT GGTTGGGATG CTGGTACGCA |
| 6421 | GGTTAATGTG AAGCGTAAGA AGGGCTGATG GGTTGTGGCT CAGGATGTGA ATGTGAAGCT |
| 6481 | GAACTTGCCG GGTATTCGTG AGGTGTTGAA GTCTTCTGGG GTGCAGTCGA TGTTGGCTGA |
| 6541 | GCGTGGCGAG CGGGTGAGGC GTGCGGCTTC GGCGAATGTT GGCGGTAATG CTTTTGATAG |
| 6601 | GGCCCAATAC CGTAGTGGTT TGTCGTCGGA GGTGCAGGTT CACCGTGTGG AGGCTGTGGC |
| 6661 | GAGGATTGGC ACCACCTATA AGGGTGGGAA GCGTATTGAG GCGAAGCATG GCACGTTGGC |
| 6721 | GAGGTCGATT GGGGCTGCGT CGTGATCGTT TACGGTGATC CGCGTGTGTG GGCTAAACGT |
| 6781 | GTGCTCAAGG ATGATGGCTG GCTGTCCGAT ATACCCTGTG TGGGGACGGT GCCTGACGAT |
| 6841 | TTCAGCGGTG ACCTGATTTG GTTGGCGTTG GATGGCGGCC CACAGTTGCA TGTTCGCGAG |
| 6901 | CAGGTGTTTT TGCGGGTGAA CGTGTTTTCT GATATGCCTG ATCGTGCCAT GTCGCTAGCC |
| 6961 | AGGCGGGTTG AGGCTGTCCT TGTAGACGGT GTGGACGGTG ACCCGGTGGT GTTTTGTCGA |
| 7021 | CGGTCTACTG GCCCTGATTT GCTGGTTGAT GGTGCACGTT TTGATGTGTA TTCGCTGTTT |
| 7081 | GAGCTGATAT GCAGGCCTGT CGAATCCGAG TAAACGTTTT GTTTTGATAT TGTTGTTTGT |
| 7141 | TTTTTGTTTG ATATTGTTTT TGGGGGTTAT GATGGCTGGA ACACGTAAAG CGTCTAATGT |
| 7201 | TCGTTCCGCG GTTACGGGTG ACGTCTATAT TGGTAAAGCT CATGCCGGTG ACACTATTGA |
| 7261 | TGGTGTGAAG ACGGTTCCTG ACGGGCTTAC AGCTTTAGGG TATCTGTCTG ATGACGGGTT |
| 7321 | TAAGATTAAA CCGGAGCGTA AAACGGATGA TTTGAAGGCT TGGCAGAATG CGGATGTTGT |
| 7381 | TCGCACTGTG GCTACGGAAT CGTCTATCGA GATTTCTTTC CAGCTGATCG AGTCTAAGAA |
| 7441 | GGAGGTTATC GAGCTGTTTT GGCAGTCGAA GGTTACTGCC GGAGCCGATT CGGGTTCGTT |
| 7501 | CGATATTTCT CCTGGTGCCA CGACGGGTGT TCATGCCCTG TTGATGGATA TTGTTGATGG |
| 7561 | CGATCAGGTT ATTCGCTACT ATTTCCCTGA GGTTGAGTTG ATCGATCGTG ACGAGATTAA |
| 7621 | GGGTAAGAAT GGCGAGGTGT ATGGGTATGG TGTGACGTTG AAGGCGTATC CTGCCCAGAT |
| 7681 | TAATAAGAAG GGTGATGCGG TGTCTGGTCG GGGGTGGATG ACGGCTTTAA AAGCTGATAC |
| 7741 | TCCTCCGACT CCTCCTCCGG CCCCGAATCC TCCGAAGCCT GAGCCGGATC CGAATCCGCC |
| 7801 | GTCTAATAAC TGATACACAT AGTTTGAGGG ATTGTTGATA GATGAGTGAC ACGGGTTACA |
| 7861 | CGTTGAAGAT TGGTGACCGT AGCTGGGTGT TGGCGGATGC GGAGGAGACG GCTCAGGCTG |
| 7921 | TTCCTGCCCG CGTTTTCCGT CGTGCTGCTA AGATTGCCCA GTCGGGTGAG TCTGCGGATT |
| 7981 | TCGCCCAGGT TGAGGTGATG TTTTCTATGT TGGAGGCTGC CGCCCCGGCT GACGCGGTGG |
| 8041 | AGGCCCTGGA GGGGCTTCCT ATGGTTCGTG TGGCCGAGAT TTTCCGCCAG TGGATGGAAT |
| 8101 | ACAAGCCTGA CGGTAAGGGT GCCTCGCTGG GGGAATAGTT TGGCTCCACG GCCTGATTGA |
| 8161 | TGATTATCGT GGGGCCATCG AATACGATTT CCGCACCAAG TTTGGTGTTT CTGTTTATAG |
| 8221 | TGTTGGTGGC CCGCAGATGT GTTGGGGTGA GGCTGTCCGG CTGGCTGGCG TGTTGTGTAC |
| 8281 | CGATACGTCT AGCCAGTTGG CGGCCCACCT GAATGGTTGG AAGCGCCCGT TTGAGTGGTG |
| 8341 | CGAGTGGGCT GTGTTGGACA TGCTGGATCA TTACAGGTCT GCTAATAGTG AGGGGCAGCC |
| 8401 | GGAGCCTGTG GCGAGGCCTA CGGATGAGCG TAGGGCCCGG TTTACGTCTG GGCAGGTGGA |
| 8461 | CGATATTTTG GCGCGTGTTC GTGCTGGTGG CGGGGTGTCT CGCGAGATTA ATATTATGGG |
| 8521 | GTGAATAGTG TATGTCTGGT GAGATTGCTT CCGCATATGT GTCGTTGTAT ACGAAGATGC |
| 8581 | CTGGTTTGAA GGCGGATGTT GGTAAACAGC TTTCTGGGGT GATGCCTGCT GAGGGTCAGC |
| 8641 | GTTCGGGTAG TTTGTTTGCT AAGGGAATGA AGTTGGCTCT TGGTGGTGCG GCGATGATGG |
| 8701 | GTGCCATCAA TGTTGCTAAG AAGGGCCTCA AGTCGATTTA TGATGTGACT ATTGGTGGCG |
| 8761 | GTATTGCTAG GGCGATGGCT ATTGATGAGG CTCAGGCTAA GTTGACTGGT TTGGGTCATA |
| 8821 | CGTCTTCTGA CACGTCTTCG ATTATGAATT CGGCTATTGA GGCTGTTACT GGTACGTCGT |
| 8881 | ATGCGTTGGG GGATGCGGCG TCTACGGCTG CGGCGTTGTC TGCTTCGGGT GTGAAGTCTG |
| 8941 | GCGGGCAGAT GACGGATGTG TTGAAGACTG TCGCCGATGT GTCTTATATT TCGGGTAAGT |
| 9001 | CGTTTCAGGA TACGGGCGCT ATTTTTACGT CTGTGATGGC TCGCGGTAAG TTGCAGGGCG |
| 9061 | ATGACATGTT GCAGCTTACT ATGGCGGGTG TTCCTGTCCT GTCTTTGCTT GCCAGGCAGA |
| 9121 | CTGGTAAAAC GTCTGCTGAG GTGTCGCAGA TGGTGTCAAA GGGGCAGATT GATTTTAACA |
| 9181 | CGTTTGCGGC TGCGATGAAG CTTGGCATGG GTGGTGCTGC GCAGGCGTCT GGTAAGACGT |
| 9241 | TTGAGGGCGC TATGAAGAAT GTTAAGGGCG CCCTGGGTTA TCTTGGTGCT ACGGCTATGG |
| 9301 | CCCCGTTTCT TAACGGGTTG CGGCAGATTT TTGTTGCGTT GAATCCGGTT ATCAAGTCTG |
| 9361 | TCACGGATTC CGTGAAGCCG ATGTTTGCTG CCGTCGATGC TGGTATTCAG CGTATGATGC |
| 9421 | CGTCTATTTT GGCGTGGATT AACCGTATGC CGGCTATGAT CACTCGAATG AATGCACAGA |
| 9481 | TGCGCGCCAA GGTGGAGCAG TTGAAGGGCG TTTTTGCAAG GTTGCATTTG CCTGTTCCTA |
| 9541 | AGGTGAATTT GGGTGCCATG TTTGCTGGCG GCACCGCAGT GTTCGGTATT GTTGCTGCGG |
| 9601 | GTGTTGGGAA GCTTGTCGCG GGGTTTGCCC CGTTGGCGGT GTCGTTGAAG AATCTGTTGC |
| 9661 | CGTCGTTTGG TGCTTTGAGG GGTGCCGCCG GGGGGCTTGG TGGCGTGTTT CGCGCCTTGG |
| 9721 | GTGGCCCTGT TGGTATTGTG ATCGGCTTGT TTGCTGCCAT GTTTGCTACG AACGCCCAGT |
| 9781 | TCCGTGCCGC TGTTATGCAG CTTGTGGGGG TGGTTGGCCG GGCTTTGGGG CAGATTATGG |
| 9841 | TCGCCTTGCA GCCATTGTTC GGGATTGTTG CTGGCGTGGT TGCCAGGTTG GCTCCCGTTT |
| 9901 | TTGGCCAGAT TATTGGTATG GTTGCTGGTT TGGCTGCCCG GCTGGTGCCT GTTATTGGTA |
| 9961 | TGCTTATTGC CCGGCTGGTT CCTGTTATCA CCCAGATTAT TGGTATGGTA ACCCAGGTTG |
| 10021 | CTGCCATGTT GTTGCCTATG CTGATGCCGG TTATTCAGGC TGTTGTTGCT GTGATACGGC |
| 10081 | AGGTTATTGG TGTGGTCATG CAGTTGATAC CTGTTTTGAT GCCGGTTGTG CAGCAGATTT |
| 10141 | TGGGTGCTGT CATGTCTGTT TTGCCGCCGA TTGTTGGTTT GATACGGTCG CTGATACCGG |
| 10201 | TGATCATGTC GATTATGCGT GTGGTGGTGC AGGTTGTTGG TGCCGTGCTA CAGGTGGTGG |
| 10261 | CCCGTATTAT TCCGGTTGTT ATGCCGATTT ATGTTTCGGT GATTGGATTC ATTGCCAAGA |
| 10321 | TTTATGCTGC GGTTATCGTT TTTGAGGCTA AGGTTATTGG CGCTATTCTT CGTACTATTA |
| 10381 | CGTGGATTGT GAATCATTCA GTGTCTGGCG TGAGGTCTAT GGGCACGGCC ATCCAGAATG |
| 10441 | GCTGGAATCA TATCAAATCG TTTACGTCGG CGTTTATTAA CGGTTTCAAG TCGATCATTT |
| 10501 | CTGCCGGTGT TGCCGCGGTT GTGGGGTTTT TTACGCGGCT TGGTTTGTCG GTTGCCTCCC |
| 10561 | ATGTGAGGTC TGGTTTTAAC GCGGCCCGTG GTGCTGTTTC TTCTGCGATG AATGCTATTC |
| 10621 | GGAGTGTTGT GTCTTCGGTG GCGTCTGCTG TTGGCGGGTT TTTCGGGTCG ATGGCGTCTA |
| 10681 | GGGTTCGTAG TGGTGCTGTG CGCGGGTTTA ATGGTGCCCG GAGTGCGGCT TCTTCTGCTA |
| 10741 | TGCATGCTAT GGGGTCTGCG GTGTCTAACG GTGTGCATGG TGTGCTGGGG TTTTTCCGGA |
| 10801 | ATTTGCCTGG CAATATTAGG GGCGCCTTGG GTAGTATGGG GTCCCTGTTG GTGTCGGCTG |
| 10861 | GCCGTGATGT GGTGTCTGGT TTGGGTAACG GTATCCGGAA TGCTTTGAGT GGCCTGTTGG |
| 10921 | ATACGGTGCG TAACATGGGT TCCCAGATTG CGAACGCGGC GAAGTCTGCG CTGGGTATTC |
| 10981 | ATTCCCCGTC TCGGGTGTTT CGTGACGAGG TTGGCCGTCA GGTTGTTGCC GGTTTGGCTG |
| 11041 | AGGGGATCAC CGGGAATGCT GGTTTGGCGT TGGATGCGAT GTCTGGTGTG GCTGGCCGTC |
| 11101 | TTCCGGATGC TGTGGATGCC CGGTTTGGTG TGCGATCGTC TGTGGGCTCG TTTACCCCGT |
| 11161 | ACGACCGGTA TCGGCGTGCG AACGAGAAGA GTGTTGTGGT GAATGTGAAC GGACCCACGT |
| 11221 | ATGGGGATCC TGCCGAGTTT GCGAAGCGGA TTGAGCGTCA GCAGCGTGAC GCTTTGAATG |
| 11281 | CGTTGGCTTA CGTGTGATCG AGGGGGTGTT GTGCATGTTT ATTCCTGACC CGTCTGATCG |
| 11341 | TGCCGGTTTG ACTGTGGATT GGACTATGTT TCCGTTGGTG GGTAATGCTC CGGAGCGTGT |
| 11401 | GCTTCATTTG ACGGATTATA CGGGGTCGTC TCCGGTCATG TTGTTGAATG ATTCGTTGCG |
| 11461 | CGGCCTGGGT ATGCCTGAGG TGGAGCAGTT TTCTCAAACG CATGTTGGTG TGCATGGTTC |
| 11521 | GGAGTGGCGC GGGTTTAATG TGAAGCCTCG CGAGGTGACT TTGCCGGTGT TGGTGTCGGG |
| 11581 | TGTTGACCCG GATCCGGTGG GCGGGTTTCG TGACGGTTTT TTGAAGGCGT ATGACGCGTT |
| 11641 | GTGGTCTGCG TTTCCTCCGG GCGAGGTGGG GGAGTTGTCT GTGAAGACTC CTGCCGGTCG |
| 11701 | TGAGCGTGTG TTGAAGTGCC GGTTTGATTC GGCTGATGAC ACGTTTACGG TTGATCCGGT |
| 11761 | GAACCGTGGC TATGCGCGCT ATCTGTTGCA TTTGACAGCT TATGATCCGT TTTGGTATGG |
| 11821 | GGATGAGCAA AAGTTTCGTT TTAGTAACGC GAAGTTGCAG GATTGGTTGG GTGGCGGCCC |
| 11881 | TGTCGGCAAG AAGGGTACCG CGTTTCCTGT GGTGTTAACA CCGGGTGTGG GCTCGGGCTG |
| 11941 | GGATAACCTG TCTAATAAGG GTGATGTGCC TGCGTGGCCT GTGATTCGTG TTGAGGGTCC |
| 12001 | TTTGGAGTCG TGGTCTGTGC AGATTGATGG TTTGCGTGTG TCTTCGGACT ATCCGGTCGA |
| 12061 | GGAGTTTGAT TGGATCACTA TTGATACGGA TCCTCGCCAG CAGTCTGCGT TGTTGAACGG |
| 12121 | GTTTGAGGAT GTGATGGATC GTTTGACAGA GTGGGAGTTT GCGCCTATCC CGCCTGGCGG |
| 12181 | TTCTAAGAGT GTGAATATTG AGATGGTTGG TTTGGGTGCT ATTGTTGTGT CGGTGCAGTA |
| 12241 | CAGGTTTTTG AGGGCTTGGT GAATAGTTGA TGGCTGGTCT TGTTCCGCAT GTAACATTGT |
| 12301 | TTACACCTGA TTATCGCCGT GTGGCGCCTA TCAATTTTTT TGAGTCGTTG AAGTTGTCGT |
| 12361 | TGAAGTGGAA TGGTTTGTCG ACTTTGGAGT TGGTGGTGTC GGGGGATCAT TCGAGGCTTG |
| 12421 | ACGGGTTGAC GAAGCCGGGT GCGCGGCTGG TTGTTGATTA TGGTGGTGGC CAGATTTTTT |
| 12481 | CTGGGCCTGT GCGTAAAGTG CATGGTGTGG GTCCGTGGCG TTCTTCCCGT GTGACTATAA |
| 12541 | CGTGTGAGGA TGATATTCGG CTGTTGTGGC GTATGTTGAT GTGGCCTGTG AATTATCGTC |
| 12601 | CTGGTTTGGT TGGTATGGAG TGGCGTGCGG ACAGGGATTA TGCCCACTAT TCGGGTGCGG |
| 12661 | CTGAGTCGGT TGCTAAGCAG GTGTTGGGGG ATAATGCTTG GCGTTTTCCG CCTGGTTTGT |
| 12721 | TTATGAACGA TGATGAGAGT CGTGGCCGCT ATATTAAGGA TTTTCAGGTG CGGTTTCACG |
| 12781 | TGTTTGCCGA TAAGTTGTTG CCGGTGTTGT CGTGGGCTCG GATGACTGTC ACGGTGAACC |
| 12841 | AGTTTGAGAA TGCGAAGTTT GATCAGCGTG GTTTGTTGTT TGATTGTGTG CCTGCTGTGA |
| 12901 | CCCGGACGCA TGTGTTGACT GCCGAGTCTG GTTCGATTGT GTCGTGGGAG TATGTGCGTG |
| 12961 | ACGCCCCGAA GGCTACTTCG GTGGTGGTTG GTGGCCGCGG CGAGGGCAAA GATCGGCTGT |
| 13021 | TTTGCGAGGA TGTTGATTCG ATGGCCGAGG ATGACTGGTT TGATCGTGTC GAGGTGTTTA |
| 13081 | AGGATGCCCG TAACACGGAT TCCGAGAATG TGCATCTTAT TGATGAGGCT GAGCGGGTGT |
| 13141 | TGTCCGAGTC GGGGGCTACG TCGGGGTTTA AGATCGAGTT GGCTGAGTCG GATGTGTTGC |
| 13201 | GGTTTGGGCC TGGCCGCCTG ATGCCGGGTG ATCTTATCTA TGTGGATGTG GGCTCGGGGC |
| 13261 | CTATTGCGGA GATTGTGCGC CAGATTGATG TGGAGTGTGA TTCGCCTGGT GATGGGTGGA |
| 13321 | CGAAGGTGAC TCCGGTTGCT GGGGATTATG AGGATAATCC GTCGGCGCTG TTGGCTCGCC |
| 13381 | GTGTGGCTGG TTTGGCTGCG GGTGTGCGGG ATTTGCAAAA ATTCTAATTG TTAGGGGTTT |
| 13441 | GTTGTGGGTA TTGTGTGTAA AGGGTTTGAT GGTGTGTTGA CCGAGTATGA TTGGGCTCAA |
| 13501 | ATGTCTGGTC TGATGGGTAA TATGCCGTCC GTGAAAGGGC CGGATGATTT TCGTGTCGGC |
| 13561 | ACTACGATTC AGGGTTCCAC GGTGTTGTGT GAGGTCCTGC CGGGGCAGGC TTGGGCTCAC |
| 13621 | GGGGTGATGT GCACGTCGAA TGCTGTTGAG ACGGTGACAG GTCAGCTTCC GGGCCCGGGT |
| 13681 | GAGACCCGCT ACGACTATGT TGTCCTGTCG CGGGATTGGC AGGAGAATAC GGCCAAGTTG |
| 13741 | GAGATTGTTC CTGGGGGGCG TGCGGAGCGT GCCCGTGACG TGTTGCGTGC GGAGCCTGGC |
| 13801 | GTGTACCATC AGCAGTTGTT GGCTACTTTG GTGGTGTCGT CTAACGGGTT GCAGCAGCAG |
| 13861 | CTTGACAGGA GGGCTATAGC GGCCCGTGTG GCGTTTGGGG AGTCTACTGC ATGTGATCCT |
| 13921 | ACCCCTGTGG AGGGTGACCG GGTGATGGTG CCTTCTGGGG CTGTGTTGGC TAATCATGCT |
| 13981 | AACGAGTGGA TGCTGTTGTC TCCGCGGATT GAGACGGGCA CTAAGTCGAT CATGTTTGGC |
| 14041 | GGGTCTGCTG TGTATGCTTA CACGATTCCG TTTGATCGCC AGTTTGCTAG TCCGCCTGTT |
| 14101 | GTGGTGGCGT CTATGGCTAC GGCGGCTGGG GGCACGACCC AGATTGATGT GAAAGCCTAC |
| 14161 | AATGTGACTG CCCAAAATTT TAGTTTGGCG TTTATTACGA ATGATGGTTC GAAGCCGAAT |
| 14221 | GGTGTGCCTG CGGTGGCTAA TTGGATTGCT GTCGGCGTGT GACTGTACAG GTGTTGTGGC |
| 14281 | GGATGGTGTG ATGTTGGGGG GCTGTGGTGT CGTGGTTTAC TCCTGCACTG GTGGCCTCTA |
| 14341 | TTTGTACCGC GTTGGCCACG GTTTTGGGTT CTGTTCAGGC TGTCACGTCT AAATCTAGGA |
| 14401 | GGCGTTTGCG CCGCCTGTCG GCGCAGGTGG ATGCGATGGA AGAGTATACG TGGGGTGTGC |
| 14461 | GGCGCGAGGT GCGAAGGTTT AACGCCGGGC TTCCTGACGA GGTGGAGCCT ATGCATCTCC |
| 14521 | CTGATTTGCC CGAGTTTTTG AAAGATACTG TTGATGGTGG AGGTGAGTAG GGTTGAGGGA |
| 14581 | GTTGGAGGAG GAGAAGCGGC AGCGCCGCAA TTTTGAGAAG GCTTCACTGG TGTTGCTGTT |
| 14641 | TTTGTCGCTT GTGTTATTGG CTGTGGTTGC TGCGGGTGCT TTGCGTTTCG GGGCTGTATC |
| 14701 | CTCTGAGCGG GATTCGGAGC AGGCGAGGGC CCAGTCGAAT GGTACAGCCG CCAAGGGTTT |
| 14761 | AGCCAGCAGT GTGCGGCAGG TGTGTGCTCA GGGTGGACGG GAGTCTGTGC GGCTTCACCA |
| 14821 | GTCTGGTTTG TGTGTGGATG CTCAGCGTGT TGAGCGTAGT GTGCAGGGTG TGCCGGGTCC |
| 14881 | TGCCGGTGAG CGCGGCCCGC AAGGCCCGGC AGGTGTGGAC GGCCGGGATG GTGTTAATGG |
| 14941 | TTCGGCTGGG CTGGTTGGCC CTGTGGGTCC GCAGGGGTCC CCGGGTTTGA ATGGTGTGAA |
| 15001 | AGGTCCTGAC GGGTTGCCTG GCGCTAACGG TTCGGATGGC CGTGATGGTG TGGACGGTGT |
| 15061 | GAACGGCAAT GATGGCGCTG ATGGTCGGGA TGGTTCGGCC GGTGAGCGCG GTGATGTGGG |
| 15121 | CCCCTCAGGT CCTGCCGGCC CGCAAGGTGC ACAGGGTGAA CGGGGTGAGC GCGGCCCCGC |
| 15181 | CGGTGCGAAT GGCACGAATG GCAAGGACGG TAAGGATGGT GCCGACGGCC GTGATGGGCG |
| 15241 | TTCGGTTGTG TCTGTGTACT GTTTCGGTGG CCTGCCAGGG TGTGAAACCA TCACCTGTGG |
| 15301 | TTACCGTGTC ATCCCGTAAA TAGAAGAAGA GGGAAGGGTG TTACTAGTGT TGATTGTGGT |
| 15361 | TTTTGGTGGT GGTGTGTGGT GAGATACATT CCTGCAGCGC ATCACTCTGC CGGCTCTAAT |
| 15421 | AATCCGGTGA ACAGGGTTGT GATTCATGCA ACATGCCCGG ATGTGGGGTT TCCGTCCGCC |
| 15481 | TCACGTAAGG GGCGGGCGGT GTCTACAGCA AACTATTTCG CTTCCCCATC GTCTGGTGGT |
| 15541 | TCGGCGCATT ATGTGTGTGA TATTGGGGAG ACGGTGCAAT GCTTGTCGGA GTCTACGATT |
| 15601 | GGTTGGCATG CCCCGCCGAA TCCGCATTCT TTGGGTATCG AGATTTGCGC GGATGGGGGT |
| 15661 | TCGCATGCCT CGTTCCGTGT GCCGGGGCAT GCTTACACTC GGGAGCAGTG GCTTGATCCG |
| 15721 | CAGGTGTGGC CTGCCGTTGA GAGGGCGGCG GTGCTGTGTA GACGTTTGTG TGACAAATAT |
| 15781 | AATGTTCCGA AAAGGAAACT GTCGGCTGCC GATTTGAAGG CTGGCAGGCG GGGTGTGTGT |
| 15841 | GGCCATGTGG ATGTTACGGA TGCGTGGCAT CAGTCGGATC ATGACGATCC TGGGCCGTGG |
| 15901 | TTTCCGTGGG ACAAATTTAT GGCCGTCGTC AACGGCGGCA GTGGAGATAG TGGGGAGTTA |
| 15961 | ACTGTGGCTG ATGTGAAAGC CTTGCATGAT CAGATTAAAC AATTGTCTGC TCAGCTTACT |
| 16021 | GGTTCGGTGA ATAAGCTGCA CCATGATGTT GGTGTGGTTC AGGTTCAGAA TGGTGATTTG |
| 16081 | GGTAAACGTG TTGATGCCTT GTCGTGGGTG AAGAATCCTG TGACGGGGAA GCTGTGGCGC |
| 16141 | ACTAAGGATG CCCTGTGGAG TGTCTGGTAT TACGTGTTGG AGTGTCGTAG CCGTCTTGAC |
| 16201 | AGGCTCGAGT CTGCTGTCAA CGATTTGAAA AAGTGATGGT GGTTTGTTGT GGGTAAACAG |
| 16261 | TTTTGGTTAG GTTTGCTAGA GCGGGCGGCT AAGACTTTTG TGCAAACGTT TGTTGCTGTG |
| 16321 | TTGGGGGTGA CGGCGGGTGT CACGTATACG GCGGAGTCGT TTCGTGGTTT GCCGTGGGAG |
| 16381 | TCTGCGTTGA TTACGGCTAC GGTTGCTGCG GTCCTGTCGG TGGCTACCTC GTTTGGTAGC |
| 16441 | CCGTCGTTTG TGGCTGGTAA GCCGAAAACC ACGCCTGTGG ATGCGGGTTT GGTTCCGCCG |
| 16501 | GATGATCCCG GAATAGTGGA GCCTCACATG GTGGATGTGT CGGATCCTGG CATGATCGAG |
| 16561 | CCTGCAGATG ATGTGGATCT TGGTGTAGGC TATGTGCCGA AACATGCTGC CGAGTCGGAG |
| 16621 | GTTGGCACGG TAGAGTCGAC TGTTGCATAA GTGAATATAG ATGTGTGCCC CAGCGGTGCT |
| 16681 | GCCACGATTG TGTGGTGGTT GCCGCTGGGG CACTATTTTT GTATATTGCG GTGTGGCTAT |
| 16741 | GATTCGTTGC TGTCGATGGT GTCTTCGAGC ATCTGGTACA GGTGGAGGCA GGTAGAGATA |
| 16801 | GTTTCGCTGG CCTGGTCGAG AACGTTCCGG CCGATAACAT TTTTGTTGTT GTCGCGGTGG |
| 16861 | CGGATGATAG ACCACATGAT CTCGTCGGCT GCCGCCTGCA ATAGTTTTGC CTGGTATGCG |
| 16921 | ATTCCAGCGA GCCAGTCTAG TGCTTCCTGG CTTGCATAGG GTGTCTGGTC CTCGCTGTTG |
| 16981 | CTTGTGGGGT GTCCTGCACT GTCGCATAGC CACAGGATTT CGCTGCACTC GTCTAGCGTG |
| 17041 | TCCTGGTCTA TAGCGAGATC GTCGAGGCTG ACATTGTTGA CGGTAAGGTT CACGTTGTCG |
| 17101 | AGGGAGATGG GTACACCGTA CTGGTTTTCG ACACCGTCAA CAATGTTTTC CAATTGCTGC |
| 17161 | ATGTTGGTGG GCTGTTGTTG GACGATACGG TGTATCGCTG TGTTGAGGGT GGTGTAGGTG |
| 17221 | ATATTGTGTG TGTTGTTCAT CGTGTTATGC CATTCCTTCG TTATCGTCTG GCCTGTAGTA |
| 17281 | TGTGCTGTTT GCGTACTCGG TTAACGTCAT CAGTGTTTGG TCTGCCCACT GTTTCACAGT |
| 17341 | CTGCCTTGTC ACTCCGAGTC GTTGGGCGGC TGTGGCGTAG GTTTGGTCAT ACCCGTATAC |
| 17401 | TTCCCTGAAT GCTGCCAACC GTGCCAAATG TTTTCGCTGT TTGGATGGCT GGCAGGCGAG |
| 17461 | GGTGTAGTCG TCGATGGCTA GCTGTAGATC GATCATGGTG GCAATGTTGT TGCCGTGGTG |
| 17521 | TTGTGGCGCG GTTGGTGGGG GTGGCATTCC TGGCTCCACA CTGGGTTTCC ATGGGCCTCC |
| 17581 | GTTCCAGATC CATTGGGCGG CTTGGATGAT GTCTGCGGTG GTGTAGGTTC GGTTCACTGG |
| 17641 | TCATCCCCTG AACAGGTTGT CTGGGTTGCT GGTGCGGATT GTGTCGAATC GTCCGACGCA |
| 17701 | GTGGCAGTAG TCGTACATGA GTTTGATAAT GTGTTGGTGG TCTCCCAAAT AGGTGTTTCC |
| 17761 | GCTGATGCTG TAGGTGGCTG TGCCGTCTTT ACTAATAGTG TATTTGGCGG TGATGGTTTC |
| 17821 | GGGGTTTTCG GTGTCGGTGA TGATGGCTGT GGTGGTGGTG CCTACGGTTT GGAGCACGGT |
| 17881 | GGTTTGGGTT CCGTCGTCGA TGGTGGTTTT AACCATGAGG TGTGTTCTCC CTTTGTGTTA |
| 17941 | GTTGCTGGTT TGGTTGTCGG CTAGATGAAT GATGTCGGGT AAGGGTTTCG GCTGGTCTAA |
| 18001 | ATGTTGTGTG GTTTTGTTGG CTAGCCGTTT GGCTACCCTG TAGCACATTT TGGTGTAGTG |
| 18061 | TTTGTTGTCT AGGTTGTGGT ATTGTTCCCG CACCGCAATA TATAGCAGGG AGTCTTGGTA |
| 18121 | CAGGTCGTCT GCATTGATTG CGGGGTAGTG TGCGGCTGTT TTAGTGCATG CCCGGTTGAG |
| 18181 | TGTGCGTAGA TGATGGTCTG TGGCCCACAC CCACGATGCG GTGGTGGCTA GGTCGGCTTT |
| 18241 | TGTTGGTCGT CGGCTCATGG CATCTCTTTC ATCTGGCTAT CTGGTAGTTG TTTGGTGTTT |
| 18301 | TGTTGTTGAT AGTGTAGCAC ACGAGTCCGG GGTTTCCGGT GGTGCCCGTC TTGTGCCGGT |
| 18361 | ACCATGTGGA TTCGCCTTCC ATGGATGGGC ATTGGATGAA GGTGCGTTGT CCTTGTTCGG |
| 18421 | AGATTTCTAG GTGGTGCCTG TGTCCGGCCA TGAGGATGTG GGATGTGGTG CCGTTGTGGA |
| 18481 | ATTCTTGTCC GCGCCACCAA TCATAGTGTT TGCCGGTGCG CCATTGGTGG CCGTGGGCGT |
| 18541 | GTAGTATCCG TGTGCCGGCT ACTTCGACGG TGGTGGTCAT TTCGTCTCGG CTGGGGAAAT |
| 18601 | AAAAGTGTAG GTTGGGGTAT TGGTTGGTGA GCTGGTAGGC TTCTGCGATG GCGCGGCAGC |
| 18661 | AGTCTACGTC GAAGGAGTCG TCGTAGGTGG TGACTCCTTT GCCGAAGCGT ACGGCTTCTC |
| 18721 | CGTGGTTGCC GGGGATGGAT GTGATGGTCA CGTTTTTGCA GTGGTCGAAC ATGTGGATGA |
| 18781 | GTTGCATCAT GGCCATGCGG GTGAGCCTGA TTTGTTCCGT CAAGGGGGTT TGTGTGCGCC |
| 18841 | AGGCGTTGTT GCCTCCTTGT GACACGTATC CTTCGATCAT GTCGCCGAGG AATGCGATGT |
| 18901 | GGACTCGTTC GGGTTTGCCT GCCTGCTGCC AGTAGTGTTT AGCTGATGTG AGGGAGCGCA |
| 18961 | GGTAGTCGTC GGCGAAGTGT GATGTTTCCC CGCCGGGGAT GCCTTTGCCG ATTTGGAAGT |
| 19021 | CGCCTGCCCC GATGACGAAG GCCGCAGTGC TGTAGTCGGT GCGGGTGTCC TGTTCGGGTT |
| 19081 | TTGGGGGTGT CCATTCGGCT AGTTTATCGA CGAGTTCGTC TACAGGGTAG GGGTTTGTTG |
| 19141 | CGGGTTGGTG GTCGATGATT TTTTGTACGG ATCTGCCTGT TTCTCCGTTG GGGAGTGTCC |
| 19201 | ATTCGGAGAT GCGTGTGCGG CGTACGGTGC CGTTTGCGAG ATCATCGCAG ATGGTGTCTG |
| 19261 | CTTCGCTATC GTGGTTGGCT AGCTGGGTGA GTAGCCGGTC TATGTTGTCT ATCACTGGGT |
| 19321 | ATCCTCTTCT TGCGGGGTGG TGTTGGCTTG TTTGCGGCGG TAGTCTTTTA TAACGGTGGC |
| 19381 | GGAGATGGGG TATCCTGCCT GGGTGAGCTG TTTTGCTAGC CATGAGGCGG GGATGGTTTT |
| 19441 | GTCGGCGAGC ACGTCGGCAG CCTTGTTGCC GTAGCGTTGG ATGAGTGTTT CAGTTTTGGT |
| 19501 | TGCCATGGTG TCCTATCGGT TGTGTGGTGG GCTGCCATCC TGTGCGGCAG TCGCCGTCGT |
| 19561 | GGCCTGGTTT GCGTGTGCAC CACGATACGG TTCTGTCTGT GTGGTTGAGT GTTTTGCCGC |
| 19621 | ACATGACGTT TTGTAGATGC TCTGGCAGTG CGCCGTCACC CTGGTTGCTG GTTTGTGTGT |
| 19681 | CGAAGAGTGT TTTCTGGTTG GTGAAATGCT CGGACACGGT GCCATTATGT ACGGGTAGTA |
| 19741 | TCCATGTTTT CCATTGTTGT TGTAGCCGGG TGTTCCAGTG GAATTGTTTT GCTGCGTTCG |
| 19801 | TGGCTTGTTT GATGGTTTTG TAGTAGCCGA CGAGGATGCG CTGGTGTTCA CTGTCGGGAG |
| 19861 | GGTTTTGGCC TCGCCAGTAT TGTGCCGCCA CGGCGTAGCG GTTGCTGGCT GTGAAGGCGT |
| 19921 | CCCAGCAGTA TTCAATAATG TGTTGTAGTA CACTATCGGG CATGTCTCGT ACTTGGTTTT |
| 19981 | CGTCGAGCCA CGCGTCGACA ATGATGTTGC GTATGGCGCG TTTGTCTTTG GTGGTGGGTT |
| 20041 | TGAATGCGAT GCTCACAGTA CGGGCCTGTC GTCTTGCATG AAATCATTAA AGGATGATTC |
| 20101 | GCTTGCGCGG CGTGCTTGTG TGATTTGCTG GTCAGACCAG TCGGGGTGTT GCTGTTTCAG |
| 20161 | ATAGTACCAG TGGCACGCAT TGTAGGTTTC GTCTTGTAGC CGGGTGAGAT GGTTTTCGGT |
| 20221 | GATGATTTGT TTCCACATAG TCCATGACAC GTCGAGCCGG TCCAATATTT CCATTGCTGG |
| 20281 | AATGTTGAAC TGGTTCAGGA AGAGTATTTC GTGGGTGTAG TATTCCTTCT CGTACTGGTC |
| 20341 | CCATCCACTT CGGTGCCTGT TGGGCTGGTT TTTGGGGTAG GCTTCCCGGC ATACTTTGTG |
| 20401 | CAAATGTTTG GCCATGTCGT CGGGTAGTTT AATGTCAGGG TTGGCGCGGA TCATGGATCG |
| 20461 | CATCCCATCA TAGGTGGTGC CCCAGGTGTG CATGATGTAG GTGGGGTCTT CACCATCAGC |
| 20521 | CCATTTTTCT GCACAGATGG CGAGGCGGAT GCGTCTCCTG GCTGATTGGC TGGTGTTGCG |
| 20581 | CCGGTTGGGG ATGGGGCACG TGTCGAGGGG ATCCATGATG TTTTGGTGTA CCTTTCTTGG |
| 20641 | TTTAGGTTGC TTGTGTGGTT TTATTGTAGC ACTGTGTCTA GTGCTTGTGT CAACCCTGTT |
| 20701 | TTGCCGGCCT GAAGGTAGGT GTCTGTGACA TCCCCCAGGG TGAGGGGCAC ATGGGTGGCT |
| 20761 | TGGGGGAGTG CGGCCTGGAG TGTTTGGGCC ATCTGGTGGC CCGCCTTGTC TGGGTCTGAC |
| 20821 | CAGATGTAGA TGTGGTCGTA GCCTTCAAAA AATTTGGTCC AAAAAGTTTG CCACGAGGTT |
| 20881 | GCGCCGGGTA GGGCTACGGC TGGCCATCCG CATTGTTCGA GGATCATGGA GTCGAATTCG |
| 20941 | CCTTCGCAAA TGTGCATTTC GGCTGCCGGG TTGGCCATGG CGGCCATGTT GTAGATGGAG |
| 21001 | CCTGTGTCTC CTGCCGGGGT TAGATATTTG GGGTGGTTGT GGGTTTTGCA ATCATGTTGG |
| 21061 | AGTGAGCAGC GGAAACGCAT TTTTCGTATT TCGGCTGGCC CTTCCCAGAC GGGGTACATG |
| 21121 | TATGGGATGG TGATGCACTG GTTGTAGTTT TCGTGGCCTT GGATGGGGTC ATTGTCGATG |
| 21181 | TATCCAAGGT GGTGGTAGCG GGCTGTTTCT TCGCTGATGC CTCTTGCCGA GAGCAGGTCG |
| 21241 | AGTATGTTTT CGAGGTGGGT TTCGTAGCGG GCTGAGGCTT TCTGGATTCG GCGGCGTTCC |
| 21301 | GCAATGTTGT AGGGGCGTAT GCTGTCGTAC ATTCGGGTTT TCTTCCTCTA ATCGTTGTTT |
| 21361 | CAGTTTGTGG AGTCCGCCTC CGATACCGCA TGTGTGGCAG TACCAGACGC CCTTGTCGAG |
| 21421 | GTTGATGCTC ATGGAGGGCT GGTGGTCGTC GTGGAACGGG CAGAGGATGT GTTGCTCGTT |
| 21481 | CCGTGACGGG TTGTAGCGTA TCTGGTGGGC GTCTAGGAGG CGGCAGGTGT CAGAGGTGTG |
| 21541 | GGAGGAGCTC GTTGAGGGTT GATACCACAT AGGCTTCGCT CCAGGGTTTG TTGCGCTGTT |
| 21601 | TCATGATGAC GAGTCCGATG GTGGATTGGT TTTCGCGGTT TCGGTGTGTT TCGTAGTTGC |
| 21661 | GTGCCTCCCG GCTGGCTTGT TTCACGAATT CGGCTAGGTG TGCCTGTCCT GCTTTGGCTT |
| 21721 | CGATCACATA GGTTTTGTTG CCGGTTGTGA GGATGAGGTC GCCTTCGTCT TCTTTACCGT |
| 21781 | TGAGGTGGAG GCGTTCTATA TCATAGCCGG TGTCGCGTAG CTGGTGGAGG AGTCTTGTTT |
| 21841 | CCCATTCGGC GCCGGCTCGG CGGTTGCGTG CCTGTTGTGT TGACATGATA GTCCTTTATG |
| 21901 | TTCTTGTGTC ATGTTCCAGG GCTGTTTTTC TACTAGGGGC CCGAAGAATG TGTATTCGGG |
| 21961 | GTAGGCTCGT AGTCGTTCGT ATTTTGTTCC GTCTGGGCTG GATTTGCCGG TTCTCTGTTT |
| 22021 | CAGGACGGCG ATGCGTGCCT CGGCGGGGAT GGTGAGGCCG TTGCCGTTGT CTTCGCCACC |
| 22081 | ATACAGGGAG ACTCCCAATA TGAGTTGTGG TTTTTCGGAG AGGCCGTTTT TGATTTCCCG |
| 22141 | CCTAGCTGGG GGGTGTTCGA TGTCGGTGCC GGTTTTGTCG GTTGCGTGGT GGGTGACGAT |
| 22201 | GATGGTGGAG CCAGTATCTC TACCTAAGGC TGTGATCCAT TGCATGGCTT CTTGCTGTGC |
| 22261 | CTGATAGTCG GATTCGCAGT CTTGGATGTC CATCAGGTTG TCTATAACAA TAATGGGTGG |
| 22321 | GAAGGTGTTC CACATTTCCA TGTAGGCTTG CAGTTCCATG GTGATGTCTG TCCATGTGAT |
| 22381 | GGGTGACTGG AATGAGAAGG TGATGTGTCC GCCGTGGTGG ATGCTGTCTC GATAGTATTC |
| 22441 | TGGCCCGTAG TTGTCGATGT TGTGTTGTAT CTGTTGGGTG GTGTGTTGGG TGTTGAGTGA |
| 22501 | GATGATTCGT GTGGAGGCCT CCCAGGGTGT CATGTCCCCT GATATGTAGA GGGCTGGCTG |
| 22561 | GTTGAGCATC GCGGTGATGA ACATGGCTAG CCCTGATTTT TGGCTGCCGG ACCGCCCCGC |
| 22621 | GATCATGACC AAATCCCCTT TGTGGATGTG CATGTCCAGG TTGTCATACA AGGGTGCTAG |
| 22681 | TTGGGGTATG CGGGGCAGTT CGGCGGCTGT TTGGGAGGCC CTCTCGAAGG ATCTTTGGAG |
| 22741 | AGAGAGCATC GGGACCTTAA TCTATCTGTT GGTTGGGTGT GTTTTGGTGG TCAGATGGAG |
| 22801 | TCGATGTCGA TGTCAGCATC GGCGGGGGCT GTGGTGTCGT CTAGCTGGCC GTTGTCGCGT |
| 22861 | TTGTCTACAT ATTCGGCAAC CTTATCGTAG ATGGCGTCGT CGAGGGGTTT GAGGACGACC |
| 22921 | GCGTTGAACC CGTTTTTGGT GCGCACGGTG GCAAGTTTGA AGGCTTGTTC TTCGCCGAGA |
| 22981 | TATGCTTCTA GGTCGCGGAT CATGGAGTGT GGGCGGTCGT TGTTGCCGCG TGCTTTTTCG |
| 23041 | ATGATGGCGT TGGGGATGGT TTCTGGGGTG CCGTTGTTGA GATCCTGGAG GGTGTGGAAG |
| 23101 | ATTGTGACAT CAGCGTAGAT GCGGTCTGCG ACCTGTCCAC CGTAGCCTTC GGTGTTGTGT |
| 23161 | TCTACGTCGC GGATTTTGAA GGCGATGGCG GTGGCGTCCT GGTTTCGGGA GGGGTTGAAG |
| 23221 | AAGGTGCTGT TGCTGTTGTT GTGGTAGTTG GCGAGTGCCA TGATTGTGTT ATCCTTTACT |
| 23281 | GTTGTGTCTG TTTTTGTTGT CTTATATTGG TTTATCGGGT GAGGCTGTTT CGTTTGCTGC |
| 23341 | GGAAAGCCTC GGAAACGTCA CTGTTACTGG TGATGGTCTT CTTGTACTGT TTGAGTAGGT |
| 23401 | CTGCTAGCTG TGTCTTGCTG GTGGCTTTGT TTATCCGGTC GATGATGATG TCGTTTTCCT |
| 23461 | GTGATGCGAT TTTGTTGACG TAGTCTTTGG CGGCTTTATC GTATCGGTCT TGAAGCAGGA |
| 23521 | TTGCTGCGCT AGCGATGAGG GTTGCGAGAT CCCAGTCTTT GGATACGGTT TCGTCTTTCA |
| 23581 | ATCCTCCTAG CAGATCAATA ATGGATTGTT TGATGTCTTC TGCGGTGTCT CCGCGGATGA |
| 23641 | CTGTCCATGG GGCGGCATAG TCGCCACCGT ATTTGAGTGT GATAGTTAGT TTTCCGCTGT |
| 23701 | CTGTGGTGTG CTCGTCGGTC ACGTGTTTTC CTTTTCGTTG TTTTCGGCTT CTGGTGGCTG |
| 23761 | TACGGTGGTT TCTATCGGGT ATCTGTAGGC GTCTTTCCCG TTGACGGCCC AGCAGGCGTC |
| 23821 | CTTGACGGGG CATCCTTTGC AGAGTGTGGT GACGTGGGGT ACGAAGATGC CTTGGCTGAT |
| 23881 | TCCTTTCATT GCTTGACTGT ACATGGATGA TACATGCCGG TAGGTGTTGT TGTCAAGATC |
| 23941 | AATGAGTTCG GTTGCTGTGC CCTGCTCGAC TGATTGCTCG TCTCCCTTGG TGGTGGCGGG |
| 24001 | TGTCCAAAAC ATGCCTTTCG TCACATGGAT GCCGTGTTGG GCGAGCATGT ACCGGTATGT |
| 24061 | GTGCAGCTGC ATACTGTCTG CGGGTAGGCG TCCGGTTTTG AGGTCCAAAA TGAAGGTTTC |
| 24121 | GCCGGTGTCG GTGTCGGTGA ATACCCGGTC AATATATCCG ACTATTTTTG TGTCATCGTC |
| 24181 | GAGGGTGGTT TCTACCGGGT ATTCGATGCC TGGCTGGCCG TCAATAACAG CGGTGGCGTA |
| 24241 | TTCTGGGTGG TTGCGCCTCC ATGTTTTCCA GCGGTCCACA AAGGTGGGGC CGTACATCAT |
| 24301 | CCACCAATTG TAGTCTTTCT TGTGTGGCCC GCCTGACTCG CACATGTTTT TGCATATTCT |
| 24361 | GCCGGAGGGC TTTATGTTTG TGCCTTCGGA TTCGGCGAGG GCGATTTGGG TGTCGAAAAT |
| 24421 | GTTTGTGAAG GATGAGAGTT TGTCTGGCAG TGCAGGGTAT TCGGCGGGGT TGTACAGGTG |
| 24481 | TAGGTCGTAT TGTTCGGTGA TGTGGTGTAT GGCGCTTCCG GCGATGGTGG CGTACCAGGT |
| 24541 | GTGGTGTTGG GCGTGGTAGC CGTGTGCTAG GCGCCATTTT TCGCCGCATT CGGCCCACTG |
| 24601 | TGTGAGTGAA CTGTAGGAGA TGTGGCCTGG ATGGTTGATG GTTTTCGGGT ATTGTGCTAG |
| 24661 | GGGCATTACT TGTCGCCTTT GTGGGTGTTC CATGGGTTGC GGGTGTCTTT GCCGGCGTGG |
| 24721 | TGTTGCTGGT AGGCGAGGAG TGCGAGGCAG TGCCAGGCAG CGTGTGCCAG ATGCGGCAAA |
| 24781 | TGTGATTCGT TGTCGAGGTT GTTGCCTTGC TGCCATGATA ACAGGTGCCG GTAGAGGGCG |
| 24841 | TCGACACTGT GGCTCCACGG GTATCCTCCG GTCCAGTTGT TGTCGCCGTA CTTGGTGGCA |
| 24901 | CCGTAGCCTG CCACGGAGCC TAGGGCGTGC AAGGCTGCGG GGTCGATGAG GGAGAGCCTG |
| 24961 | CAGAGTTTCA ATTCTTTTCG GGCACCGCTG TTGGGGTCGG TGTACATGCT GGTGGGCTCA |
| 25021 | TCCATGGTGT GTGTGCTCCT TAAGCGTGGG TTACTGGTTA TTGTCGTGGG CGAGTGCTAC |
| 25081 | GGCGAGAATA ATGATGGCGA GGGTTTCAGC GATCAGTATG GGTGTTGTGA TCATTTAGTG |
| 25141 | TCTCGGGGAT TATTGGTGAG TGTTGATGCA CCTAGGAGGG TGGCGAGGGC GCATGCGGCG |
| 25201 | ATGGTGGCGA GGGCTGCCTT GTGTGGGGTG CCGGTTGCGT ACATCCATGT GATGATGCCG |
| 25261 | CCTTGGATCC AGGCTAGACT GGTGAAGAAC GTTTCGTAAC TGTGTAGCTC AATGTTGTTG |
| 25321 | TTGGGTGTGT TCATGCTTGC TCCTGAAGAA TGGTGTTGAT GGTTTTATAA ATGTTGTACA |
| 25381 | GGTCGGTTTC GATAGATAAC AGTTGGTTGA TTTGGTGGTC GAGATCAATG TCTGGGTTGA |
| 25441 | GGGTGTCGAT GCGGGCGGCG ATATCGGTGG CGGTGCGTAG GCTTACTGCT GCACCGTGGA |
| 25501 | TGATGTGGCA CATGTCGGTG AGGCCGACTT TGGCGATATA GTGTGACATG AGAGGCATAA |
| 25561 | TAGGTGTGCT GTCTTTCTGG TCAGCGTGAA GGGTTGATGG ACATATCCTC TACCTGTGGT |
| 25621 | TTGTCTTCGG TGCCGGAGAC TTGGCAGAAG ACTTTCACAT GCGTCTTGGA TGCTCCGGCC |
| 25681 | TGTTTGGCGG TGGCACCGTA GGCGATAGTA AAGGTGTCTT TGTGGGCGCC GATGACTTTG |
| 25741 | TGTAGGAAGA GGTCGATGTC GGGGTTGCCG TTCCATTTGA CACCGTTTTC TGCGGCTGTC |
| 25801 | TGGGTGGCTT TCTGATTGCA GGCGTGTGCG GCGGTGATCA TGGTGAGACC CTTGCTGGTT |
| 25861 | TCTTCACCCC TTGCTTGGGC TTGCCGGTGG GCTTTGGCCT GCTCGGCTTG TAGGGAGCGG |
| 25921 | ACTGCTGCGG CCTGGCGGGC CTTCTTCTCA GCCTTGCGCT GCTGGACGGT TTTGGGTGTC |
| 25981 | CATTCGGTGT TGGCTGTGGT TACCTGTGGT GCGGGTTGTG AGGCGAGTGG CGGATTGTCG |
| 26041 | TCTGGGGCTG GCATGAAGGA TGCTGCGGCA ATAATGGCGA CTGTGGCGCC TGCGATGGTG |
| 26101 | TAGCCTGTTT TCTTGTTCAT GATTTTATGT TCCCCTTTCC GGGGTGTTGT TCGTTGCTGA |
| 26161 | CATGGTTAAT ACTTTCAGCG GCTGGGCCCA CTGTCAAGGC TGCGCTCAGT TTGTGTGAGC |
| 26221 | GTTTCTTGTG TGGCTAGGGG TGATGGCTTC TTTCGCCCAA TAGGATGTGC CACCGCTGGT |
| 26281 | CCAGTATCCG AGTTTGTTGC GCTGCATGCC CTTGGCGTCC ATCTCGTCGA TAGTGAGGCA |
| 26341 | CCTGCGGCGA TTGGGGCCTG TCTTGACCCC GTGGTCGCCT GTCCGGTGCA TGTCGCCTGA |
| 26401 | GGTGGTACTC GTGAATGTTT CATGGCAGAT GGTACAGTGC TCTGGTCGAT ATCCGGTGAT |
| 26461 | TGTGCTATCG CACTTGTGGC ATGTCCATTC CATGATTGCT CCTATTTTCC ATTATAAGAC |
| 26521 | TTCCTGTAGT GCCATTTTAG CGCCTTGCGG GTCTTGGGGG TACAACTATA TAGGTCAGGT |
| 26581 | GTTTCTAGGC GATTCTAGGC TCATTGTGTG TGGCTGGGGT TTTATCGGGC ACACAGGGTG |
| 26641 | AGCAGGTGGC CAACATTGAT GCGGGTCACA TTCCAGTAGA GTTGCGTGGC TTCCCCACTG |
| 26701 | GTGAGCGGCT TCCACTCGTC ATGGCTGAAC ACGGTGCCAT CGGATGCGAT GAACGTGTTG |
| 26761 | GGGCGTAGCT TGTGGAGTTC GGCTTCCACG CTCTGCCGGT AGGCTTCGGC GAGGCCCTCA |
| 26821 | AAATCCATGT GGTCGCAGGG GAGGTTTTCG AGGCGTGTCA GGTCGAAGGG TGTGGGGCAG |
| 26881 | TCGTAGCTGG CGGGGGTGTA GAGCTGGGTG AAGTGGTTGG CGATCTTCTG CATCATGATT |
| 26941 | CCTTTTCTGA TGATGGTGTG TTGAGGGTTT ATCGGGTGGA TGCGACAAGG ATGGCGTCTA |
| 27001 | CATCGATCAT GTCGATGAGA TCGTGGAGTT CCTCGGCCTC GTTCTCAGTG AGTGGCTGCC |
| 27061 | AGGCGTAGTC GCCGTATACG GCGCCGTCGA GGGTGACAGT CCACGGGGGC CGGATGAGTC |
| 27121 | GTATGGCTTC TTGTACTTTA GCGTGGTACA TGCGGCGCAC CATATCCAGA TCGATGTCGT |
| 27181 | CTGAATGGTT TCCGGTGAGG CTGTGGAGGC TGAGCGGGTC GATGTCTGTC TGCCTGTAGA |
| 27241 | GGGATGTGAA GGATGGGGTG ATGAGTGTGC CATCCATGAG TGTGCTCCTT TCGGTGGTTG |
| 27301 | TAGGGGTTGT TGTGGTTTCT AGAGTGTGCG GGCTGCGACC CCACAGTCAA GGTGTCGCTC |
| 27361 | AAACTCAGTG AGCGTTTCAT ATGGGTGTGT TGGGTGTGAC AGATGTCACT TAAGCCTTGA |
| 27421 | TGGCCTCTCT CAGCGCCTCA AATCTTCTAG GGGTAGGATT ATGAAGGGTT GGCCCTGCTG |
| 27481 | ATCGATTCTA GGCCCCATAC AGGGCGTCTG AGGGGTGTGT CTGAGTGATA GTGGGTGTGG |
| 27541 | CAGATGATCT AGCGAGTCAA GGTGCCGAGC TGAGACATAA GATCTATCAT CTAGGTGTGT |
| 27601 | GAGATGTATC ACATCCTCCC GGCTTGGTGT GCACCCTCAA GGCCACCCAG TCGATCTGAC |
| 27661 | GTGGAGGGTG TAGCCCAGAA ATACTGTTTA AAGCCTTCAC ACGGCGCCTA GGAGCGCCTT |
| 27721 | ACAGGGTGGG GGCTAGGTAT TTATACCCCC AGCACATTCT GATCGATTCT AGACGCCTAC |
| 27781 | AGGAGCCCGA TACACGATCA GCCATCCAGA CGCAGATCAT CAGCACCTAT CATGGTTAGC |
| 27841 | TAAGCCTCAA CTATGTGGAC AGTGTTGGTT ACTGTGGGGG AAGAAGGACA CGGTAAAAGA |
| 27901 | AAGAGGGGGA GTATCAGCTT TAAAGCCTTA AGGTCTTAGC GCTTAGCACC GATGGTCTTA |
| 27961 | GCAGTTAGCA CCGAGCCCCC TCAAGGGCTC GGCATCAGCC CGAACAGGCA CAGCCATGAA |
| 28021 | AGGAGTACAC GCCATCAGGG AAGGCTTTCG AGTACGAGGA GCCTCAGCGA CGAGTACTCG |
| 28081 | AAAGCCTGAG GGAACACCCA TCAGCACTGA TGAGCCTAGC GTATTCGGAA AGGACACAAG |
| 28141 | AGTGAAGTGT GACAGCTGTC CGGGAGTGAA CCCCGTTCTG ACTAGGGGTT TCAGCCTTAA |
| 28201 | CCACCCTCAA AGGTTACAAG ACTCTAAGAA AATTTAAGGA AAAGTTTAGG TTTAATTTTT |
| 28261 | GGACCTTTAC TACCAAAAAC ACCCGTTTAC AGCCCTCAAA CCCGCCTATA GAGCCAAAAC |
| 28321 | CACCAGTTTG ACTCATCCCA GGTGGGGTAT GATAGGCTGG ACAGGTAGCC AGCTGGACGC |
| 28381 | AAGGCCGGAA AGTGCTAACG CACTTTCCAA CCTCGCTTAC CATCAGTCTA CCAAACACTT |
| 28441 | AAAGACCTAA GGGCTTAGCG CTAAGGTGCT GATAGCTTAG CACCGAGCCC CCTCAAGGGC |
| 28501 | TCGGCATCAG TCTTAAAGCC TTAAATACTT AAAGTAACTA TAAAACTTTA AAAGCTTAAC |
| 28561 | ACTTAAGGAT ATAAACTTTA CATCAGTGTT TAAGACTTAA AAACTTAAAA TAACTATTAA |
| 28621 | GACTTAAAGT AACTATAAAA CATTAAAGAC CTTAAGTACT TAAAGTTAAC CATCAGTCTT |
| 28681 | AAACTTTACT ATGATAACCT ATAAGTCTTA AAGCTTATAG GTATAATAAT ATAATATAAG |
| 28741 | TATTAAAGCT TATAAGTTAT AAAAGTTTTA GAAGAGTTAA AGGGTTAACT TCTTTACTTC |
| 28801 | TCTTCTCTCT TTGGTTCTTT CTCTCTTCTC TTCTTTTCTT CATCGGGGGA GAAGAGGAAC |
| 28861 | CTTTAACGTC AACGCTGATG GACTTTTCGC CGTGTGTCTC GTGTGCTTCT GGTCGCAAGC |
| 28921 | TCCCATCGCA CACTCCCCAC ACTCTTTCAC CTGTGTCCCT TTCAGGCTTA GCGTGTTCAG |
| 28981 | CTGAAGGCGT ACAGCGTGTC ACGCTTAAAC CCTTAACACC AGGTAAGACT TAAAGTGCAT |
| 29041 | ATTATAAGTA GAAGACTTTA AAACCTTAAG GGTGTTCCTG CTTAGCCTGT GTCCTTTAAC |
| 29101 | GCTAGGCGCT AAGCCGTGAA ACGTGAACAC CCATCCACCC CTCTTCTTTT TACCGTGTCC |
| 29161 | TTCTTCTTTT GACACCGCTG GGGGGCGATG TGATCTTTTT AACATGCCAG GGGGTGCGGG |
| 29221 | TAGAAAACAA CCACCCCACC ACAAACAGAA CACCCCCTCA AACGCACAAA ACAGCCCCCA |
| 29281 | GGATCGATGA ACAGGGCAAG GGCAAGGTAT TCATACCCCC AGACGATTCC AGGCCGTTAG |
| 29341 | AGAGGCAAAT AAGACCCGTA CAGGGCTAGG TGAGGAATAG ACACATCATG GCACGCACCA |
| 29401 | ATCGCACAGC TAGCCAAGCC CACCGACGCT GGCGGCAACG ACTCATCACC CAAGCCCAAC |
| 29461 | AACAAGGCCA AACCGAATGC CCACTCTGCG GAGTCACCAT CACCTGGGAC ACACACGACC |
| 29521 | TACCAACCAG CCCCGAAGCC GACCACATCA CACCCGTCAG CAGGGGAGGA CTCAACACCC |
| 29581 | TCGACAACGG GCAAATCATC TGCAGAACAT GCAACAGAAG CAAAGGCAAT CGCAGCGAAC |
| 29641 | CAAACATCAA ATTCCAACAA CAAACCACAA AAACATTGAT TCCATGGTGA CAAACCCGCC |
| 29701 | AACCCCCACC GGGGACACCC CCTGCACAGG CGTGCAAGAC CTCGTACGGC TT |

In embodiments, the bacteriophage is a bacteriophage as deposited under Accession No. NCIMB 41349, 41350, or 41351. In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the genome of the bacteriophage deposited under Accession No. NCIMB 41349. In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the genome of the bacteriophage deposited under Accession No. NCIMB 41350. In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the genome of the bacteriophage deposited under Accession No. NCIMB 41351.

In embodiments, the bacteriophage has a genome with a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the nucleotide sequence of SEQ ID NO: 1. In embodiments, the bacteriophage has a genome with a nucleotide sequence that is identical to the nucleotide sequence of SEQ ID NO: 1.

In embodiments, the genome of the bacteriophage encodes, from the 5′ to the 3′ end, a small terminase, a large terminase, a portal protein, gp4, a scaffold protein, a major head protein, gp7, gp8, gp9, gp10, a major tail protein, gp12, gp13, a tape measure protein, a minor tail subunit, optionally a protease, gp17, gp18, a tail protein, an amidase, a holin, gp22, gp23, a sigma factor, gp25, gp26, gp27, gp28, gp29, gp30, a DNA primase, a DNA primase 2, gp33, a DNA helicase, gp35, gp36, an exonuclease, gp38, gp39, gp40, gp41, gp42, gp43, gp44, gp45, gp46, gp47, and gp48.

In embodiments, the composition further includes a P. acnes biofilm degrading enzyme.

In embodiments, the enzyme is an anti-aging enzyme. In embodiments, the anti-aging enzyme is a superoxide dismutase or a peroxidase.

In embodiments, the enzyme is a P. acnes biofilm degrading enzyme. In embodiments, the enzyme is a glycosidase, a protease, a DNAse, or a restriction endonuclease. In embodiments, the enzyme is a glycosidase. In embodiments, the glycosidase is a glycoside hydrolase. In embodiments, the enzyme catalyzes the hydrolysis of linear polymers of N-acetyl-D-glucosamines. In embodiments, the enzyme is a β-hexosaminidase. In embodiments, the enzyme hydrolyzes β-1,6-glycosidic linkages of acetylglucosamine polymers. In embodiments, the enzyme is a DNAse I, a restriction endonuclease, papain, bromelain, Trypsin, Proteinase K, Subtilisin, serratiopeptidase, dispersin, alginate lyase, amylase, or cellulase. In embodiments, the enzyme is Dispersin B. In embodiments, the enzyme is a protease, and the protease is proteinase K or subtilisin.

In embodiments, the enzyme is a dispersin. In embodiments, the enzyme is Dispersin B. In embodiments, the enzyme is a naturally occurring form, homolog, isoform or variant of a dispersin (such as Dispersin B) that maintains the enzymatic activity (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes Dispersin B is as follows:

(SEQ ID NO: 11)
ATGAATTGTTGCGTAAAAGGCAATTCCATATATCCGCAAAAAACAAGTAC
CAAGCAGACCGGATTAATGCTGGACATCGCCCGACATTTTTATTCACCCG
AGGTGATTAAATCCTTTATTGATACCATCAGCCTTTCCGGCGGTAATTTT
CTGCACCTGCATTTTTCCGACCATGAAAACTATGCGATAGAAAGCCATTT
ACTTAATCAACGTGCGGAAAATGCCGTGCAGGGCAAAGACGGTATTTATA
TTAATCCTTATACCGGAAAGCCATTCTTGAGTTATCGGCAACTTGACGAT
ATCAAAGCCTATGCTAAGGCAAAAGGCATTGAGTTGATTCCCGAACTTGA
CAGCCCGAATCACATGACGGCGATCTTTAAACTGGTGCAAAAAGACAGAG
GGGTCAAGTACCTTCAAGGATTAAAATCACGCCAGGTAGATGATGAAATT
GATATTACTAATGCTGACAGTATTACTTTTATGCAATCTTTAATGAGTGA
GGTTATTGATATTTTTGGCGACACGAGTCAGCATTTTCATATTGGTGGCG
ATGAATTTGGTTATTCTGTGGAAAGTAATCATGAGTTTATTACGTATGCC
AATAAACTATCCTACTTTTTAGAGAAAAAAGGGTTGAAAACCCGAATGTG
GAATGACGGATTAATTAAAAATACTTTTGAGCAAATCAACCCGAATATTG
AAATTACTTATTGGAGCTATGATGGCGATACGCAGGACAAAAATGAAGCT
GCCGAGCGCCGTGATATGCGGGTCAGTTTGCCGGAGTTGCTGGCGAAAGG
CTTTACTGTCCTGAACTATAATTCCTATTATCTTTACATTGTTCCGAAAG
CTTCACCAACCTTCTCGCAAGATGCCGCCTTTGCCGCCAAAGATGTTATA
AAAAATTGGGATCTTGGTGTTTGGGATGGACGAAACACCAAAAACCGCGT
ACAAAATACTCATGAAATAGCCGGCGCAGCATTATCGATCTGGGGAGAAG
ATGCAAAAGCGCTGAAAGACGAAACAATTCAGAAAAACACGAAAAGTTTA
TTGGAAGCGGTGATTCATAAGACGAATGGGGATGAGTGA

A non-limiting example of a Dispersin B amino acid sequence is as follows:

(SEQ ID NO: 12)
MNCCVKGNSIYPQKTSTKQTGLMLDIARHFYSPEVIKSFIDTISLSGGNF
LHLHFSDHENYAIESHLLNQRAENAVQGKDGIYINPYTGKPFLSYRQLDD
IKAYAKAKGIELIPELDSPNHMTAIFKLVQKDRGVKYLQGLKSRQVDDEI
DITNADSITFMQSLMSEVIDIFGDTSQHFHIGGDEFGYSVESNHEFITYA
NKLSYFLEKKGLKTRMWNDGLIKNTFEQINPNIEITYWSYDGDTQDKNEA
AERRDMRVSLPELLAKGFTVLNYNSYYLYIVPKASPTFSQDAAFAAKDVI
KNWDLGVWDGRNTKNRVQNTHEIAGAALSIWGEDAKALKDETIQKNTKSL
LEAVIHKTNGDE

In embodiments, the enzyme is an alginate lyase. In embodiments, the enzyme is a naturally occurring form, a homolog, an isoform or a variant of an alginate lyase that maintains the enzymatic activity of the alginate lyase (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes an alginate lyase is as follows:

(SEQ ID NO: 13)
ATGAAAACGTCCCACCTGATCCGTATCGCCCTGCCCGGTGCCCTCGCCGC
GGCATTGCTCGCCAGCCAGGTCAGCCAGGCCGCCGACCTGGTACCCCCGC
CCGGCTACTACGCGGCGGTCGGCGAGCGCAAGGGCAGCGCCGGCAGCTGC
CCCGCGGTGCCGCCGCCGTATACCGGCAGCCTGGTCTTCACCAGCAAGTA
CGAAGGCTCCGATTCGGCGCGGGCGACCCTCAACGTCAAGGCGGAGAAGA
CCTTCCGCTCGCAGATCAAGGACATCACCGACATGGAGCGCGGCGCCACC
AAGCTGGTCACCCAGTACATGCGCAGCGGCCGCGACGGCGACCTGGCCTG
CGCACTGAACTGGATGAGCGCCTGGGCCCGCGCCGGCGCCCTGCAGAGCG
ACGACTTCAACCACACCGGCAAGTCCATGCGCAAATGGGCGCTGGGCAGC
CTCTCCGGCGCCTACATGCGCCTGAAGTTCTCCAGCTCGCGGCCGCTCGC
GGCCCACGCCGAGCAGAGCCGGGAAATCGAGGACTGGTTCGCCCGGCTCG
GCACCCAGGTAGTCCGCGACTGGAGCGGCCTGCCGCTGAAGAAGATCAAC
AACCATTCCTACTGGGCGGCCTGGTCGGTGATGTCCACCGCGGTGGTGAC
CAACCGCCGCGACCTCTTCGACTGGGCGGTGAGCGAGTTCAAGGTCGCCG
CCAACCAGGTCGACGAGCAGGGCTTCCTGCCCAACGAACTCAAGCGCCGC
CAGCGCGCCCTCGCCTACCACAACTATGCGCTGCCACCGCTGGCGATGAT
CGCCGCGTTCGCCCAGGTCAACGGCGTCGACCTGCGCCAGGAGAACCACG
GCGCCCTGCAGCGCCTGGCCGAGCGGGTGATGAAGGGAGTCGACGACGAG
GAAACCTTCGAGGAGAAGACCGGCGAGGACCAGGACATGACCGACCTCAA
GGTCGACAACAAGTACGCCTGGCTGGAGCCCTACTGCGCCCTCTACCGCT
GCGAGCCGAAGATGCTCGAGGCGAAGAAGGACCGCGAGCCGTTCAACAGT
TTCCGCCTCGGCGGCGAAGTGACGCGGGTGTTCAGCCGCGAAGGGGGAAG
TTG

A non-limiting example of an alginate lyase amino acid sequence is as follows:

(SEQ ID NO: 14)
MKTSHLIRIALPGALAAALLASQVSQAADLVPPPGYYAAVGERKGSAGSC
PAVPPPYTGSLVFTSKYEGSDSARATLNVKAEKTFRSQIKDITDMERGAT
KLVTQYMRSGRDGDLACALNWMSAWARAGALQSDDFNHTGKSMRKWALGS
LSGAYMRLKFSSSRPLAAHAEQSREIEDWFARLGTQVVRDWSGLPLKKIN
NHSYWAAWSVMSTAVVTNRRDLFDWAVSEFKVAANQVDEQGFLPNELKRR
QRALAYHNYALPPLAMIAAFAQVNGVDLRQENHGALQRLAERVMKGVDDE
ETFEEKTGEDQDMTDLKVDNKYAWLEPYCALYRCEPKMLEAKKDREPFNS
FRLGGEVTRVFSREGGS

In embodiments, the enzyme is an amylase. In embodiments, enzyme is a naturally occurring form, a homolog, an isoform or a variant of an amylase that maintains the enzymatic activity of the amylase (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes an amylase is as follows:

(SEQ ID NO: 15)
ATGAAACAACAAAAACGGCTTTACGCCCGATTGCTGACGCTGTTATTTGC
GCTCATCTTCTTGCTGCCTCATTCTGCAGCAGCGGCGGCAAATCTTAATG
GGACGCTGATGCAGTATTTTGAATGGTACATGCCCAATGACGGCCAACAT
TGGAAGCGTTTGCAAAACGACTCGGCATATTTGGCTGAACACGGTATTAC
TGCCGTCTGGATTCCCCCGGCATATAAGGGAACGAGCCAAGCGGATGTGG
GCTACGGTGCTTACGACCTTTATGATTTAGGGGAGTTTCATCAAAAAGGG
ACGGTTCGGACAAAGTACGGCACAAAAGGAGAGCTGCAATCTGCGATCAA
AAGTCTTCATTCCCGCGACATTAACGTTTACGGGGATGTGGTCATCAACC
ACAAAGGCGGCGCTGATGCGACCGAAGATGTAACCGCGGTTGAAGTCGAT
CCCGCTGACCGCAACCGCGTAATTTCAGGAGAACACCTAATTAAAGCCTG
GACACATTTTCATTTTCCGGGGCGCGGCAGCACATACAGCGATTTTAAAT
GGCATTGGTACCATTTTGACGGAACCGATTGGGACGAGTCCCGAAAGCTG
AACCGCATCTATAAGTTTCAAGGAAAGGCTTGGGATTGGGAAGTTTCCAA
TGAAAACGGCAACTATGATTATTTGATGTATGCCGACATCGATTATGACC
ATCCTGATGTCGCAGCAGAAATTAAGAGATGGGGCACTTGGTATGCCAAT
GAACTGCAATTGGACGGTTTCCGTCTTGATGCTGTCAAACACATTAAATT
TTCTTTTTTGCGGGATTGGGTTAATCATGTCAGGGAAAAAACGGGGAAGG
AAATGTTTACGGTAGCTGAATATTGGCAGAATGACTTGGGCGCGCTGGAA
AACTATTTGAACAAAACAAATTTTAATCATTCAGTGTTTGACGTGCCGCT
TCATTATCAGTTCCATGCTGCATCGACACAGGGAGGCGGCTATGATATGA
GGAAATTGCTGAACGGTACGGTCGTTTCCAAGCATCCGTTGAAATCGGTT
ACATTTGTCGATAACCATGATACACAGCCGGGGCAATCGCTTGAGTCGAC
TGTCCAAACATGGTTTAAGCCGCTTGCTTACGCTTTTATTCTCACAAGGG
AATCTGGATACCCTCAGGTTTTCTACGGGGATATGTACGGGACGAAAGGA
GACTCCCAGCGCGAAATTCCTGCCTTGAAACACAAAATTGAACCGATCTT
AAAAGCGAGAAAACAGTATGCGTACGGAGCACAGCATGATTATTTCGACC
ACCATGACATTGTCGGCTGGACAAGGGAAGGCGACAGCTCGGTTGCAAAT
TCAGGTTTGGCGGCATTAATAACAGACGGACCCGGTGGGGCAAAGCGAAT
GTATGTCGGCCGGCAAAACGCCGGTGAGACATGGCATGACATTACCGGAA
ACCGTTCGGAGCCGGTTGTCATCAATTCGGAAGGCTGGGGAGAGTTTCAC
GTAAACGGCGGGTCGGTTTCAATTTATGTTCAAAGATAG

A non-limiting example of an amylase amino acid sequence is as follows:

(SEQ ID NO: 16)
MKQQKRLYARLLTLLFALIFLLPHSAAAAANLNGTLMQYFEWYMPNDGQH
WKRLQNDSAYLAEHGITAVWIPPAYKGTSQADVGYGAYDLYDLGEFHQKG
TVRTKYGTKGELQSAIKSLHSRDINVYGDVVINHKGGADATEDVTAVEVD
PADRNRVISGEHLIKAWTHFHFPGRGSTYSDFKWHWYHFDGTDWDESRKL
NRIYKFQGKAWDWEVSNENGNYDYLMYADIDYDHPDVAAEIKRWGTWYAN
ELQLDGFRLDAVKHIKFSFLRDWVNHVREKTGKEMFTVAEYWQNDLGALE
NYLNKTNFNHSVFDVPLHYQFHAASTQGGGYDMRKLLNGTVVSKHPLKSV
TFVDNHDTQPGQSLESTVQTWFKPLAYAFILTRESGYPQVFYGDMYGTKG
DSQREIPALKHKIEPILKARKQYAYGAQHDYFDHHDIVGWTREGDSSVAN
SGLAALITDGPGGAKRMYVGRQNAGETWHDITGNRSEPVVINSEGWGEFH
VNGGSVSIYVQR

In embodiments, the enzyme is a cellulase. In embodiments, enzyme is a naturally occurring form, a homolog, an isoform or a variant of a cellulase that maintains the enzymatic activity of the cellulase (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes a cellulase is as follows:

(SEQ ID NO: 17)
ATGAAGTTTCAGAGCACTTTGCTTCTTGCCGCCGCGGCTGGTTCCGCGTT
GGCTGTGCCTCATGGCTCCGGACATAAGAAGAGGGCGTCTGTGTTTGAAT
GGTTCGGATCGAACGAGTCTGGTGCTGAATTTGGGACCAATATCCCAGGC
GTCTGGGGAACCGACTACATCTTCCCCGACCCCTCGACCATCTCTACGTT
GATTGGCAAGGGAATGAACTTCTTCCGCGTCCAGTTCATGATGGAGAGGT
TGCTTCCTGACTCGATGACTGGTTCATACGACGAGGAGTATCTGGCCAAC
TTGACGACTGTGGTGAAAGCGGTCACGGATGGAGGCGCGCATGCGCTCAT
CGACCCTCATAACTATGGCAGATACAACGGGGAGATCATCTCCAGTACAT
CGGATTTCCAGACTTTCTGGCAGAATCTGGCGGGCCAGTACAAAGATAAC
GACTTGGTCATGTTTGATACCAACAACGAATACTACGACATGGACCAGGA
TCTCGTGCTGAATCTCAACCAAGCAGCCATTAACGGCATCCGCGCTGCAG
GTGCAAGCCAGTACATTTTCGTCGAAGGCAACTCCTGGACCGGAGCTTGG
ACATGGGTCGATGTCAACGATAATATGAAGAATTTGACCGACCCAGAAGA
CAAGATCGTCTATGAAATGCACCAGTACCTAGACTCCGACGGTTCCGGCA
CTTCGGAGACCTGTGTCTCCGGGACAATCGGAAAGGAGCGGATCACTGAT
GCTACACAGTGGCTCAAGGACAATAAGAAGGTCGGCTTCATCGGCGAATA
TGCCGGGGGGTCCAATGATGTGTGTCGGAGTGCCGTGTCCGGGATGCTAG
AGTACATGGCGAACAACACCGACGTATGGAAGGGTGCGTCGTGGTGGGCA
GCCGGGCCATGGTGGGGAGACTACATTTTCAGCCTGGAGCCCCCAGATGG
AACTGCTTACACGGGTATGCTGGATATCCTGGAGACGTATCTCTGA

A non-limiting example of a cellulase amino acid sequence is as follows:

(SEQ ID NO: 18)
MKFQSTLLLAAAAGSALAVPHGSGHKKRASVFEWFGSNESGAEFGTNIPG
VWGTDYIFPDPSTISTLIGKGMNFFRVQFMMERLLPDSMTGSYDEEYLAN
LTTVVKAVTDGGAHALIDPHNYGRYNGEIISSTSDFQTFWQNLAGQYKDN
DLVMFDTNNEYYDMDQDLVLNLNQAAINGIRAAGASQYIFVEGNSWTGAW
TWVDVNDNMKNLTDPEDKIVYEMHQYLDSDGSGTSETCVSGTIGKERITD
ATQWLKDNKKVGFIGEYAGGSNDVCRSAVSGMLEYMANNTDVWKGASWWA
AGPWWGDYIFSLEPPDGTAYTGMLDILETYL

In embodiments, the enzyme is proteinase K. In embodiments, the enzyme is a naturally occurring form, a homolog, an isoform or a variant of proteinase K that maintains the enzymatic activity of proteinase K (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes proteinase K is as follows:

(SEQ ID NO: 19)
ATGCGTTTGTCTGTTCTTCTGAGTCTTCTTCCCCTCGCTCTCGGCGCTCC
TGCCGTTGAGCAGCGCTCCGAGGCTGCTCCTCTGATCGAGGCCCGCGGCG
AGATGGTTGCCAACAAGTACATTGTCAAGTTCAAGGAGGGTAGCGCTCTT
TCTGCTCTCGATGCTGCCATGGAGAAGATTTCTGGCAAGCCCGACCACGT
CTACAAGAACGTCTTCAGTGGTTTCGCTGCGACCCTTGACGAGAACATGG
TTCGGGTTCTCCGCGCCCATCCCGATGTTGAGTACATTGAGCAGGATGCT
GTTGTCACCATCAACGCTGCGCAGACCAACGCTCCCTGGGGCCTTGCTCG
CATCTCCAGCACCAGCCCCGGTACCTCTACTTACTACTATGACGAATCTG
CCGGCCAAGGCTCCTGCGTCTACGTGATTGACACCGGTATCGAGGCATCG
CACCCCGAGTTTGAGGGTCGTGCCCAGATGGTCAAGACCTACTACTACTC
CAGTCGCGACGGTAACGGTCACGGCACTCACTGCGCTGGTACCGTTGGCT
CCCGAACCTACGGTGTCGCCAAGAAGACCCAGCTCTTTGGTGTCAAGGTC
CTCGATGACAACGGCAGTGGCCAGTACTCCACCATCATCGCCGGTATGGA
CTTTGTTGCCAGCGACAAGAACAACCGCAACTGCCCCAAAGGTGTCGTTG
CCTCCTTGTCCCTTGGCGGTGGTTACTCCTCCTCCGTGAACAGCGCCGCT
GCCAGGCTCCAGAGCTCTGGTGTCATGGTCGCCGTCGCTGCCGGTAACAA
CAACGCTGACGCCCGCAACTACTCCCCTGCTTCTGAGCCCTCGGTCTGCA
CTGTCGGTGCTTCTGACCGCTACGACAGACGCTCCAGCTTCTCCAACTAC
GGCAGCGTTTTGGACATCTTTGGCCCTGGTACCAGCATTCTCTCCACCTG
GATCGGCGGCAGCACCCGCTCCATCTCTGGAACTTCCATGGCTACTCCCC
ACGTTGCCGGTCTCGCTGCCTACCTCATGACTCTTGGAAAGACTACCGCC
GCCAGCGCTTGCCGATACATTGCCGACACCGCCAACAAGGGCGACTTGAG
CAACATTCCCTTCGGCACTGTCAACCTGCTTGCCTACAACAACTACCAGG
CTTAA

A non-limiting example of a proteinase K amino acid sequence is as follows:

(SEQ ID NO: 20)
MRLSVLLSLLPLALGAPAVEQRSEAAPLIEARGEMVANKYIVKFKEGSAL
SALDAAMEKISGKPDHVYKNVFSGFAATLDENMVRVLRAHPDVEYIEQDA
VVTINAAQTNAPWGLARISSTSPGTSTYYYDESAGQGSCVYVIDTGIEAS
HPEFEGRAQMVKTYYYSSRDGNGHGTHCAGTVGSRTYGVAKKTQLFGVKV
LDDNGSGQYSTIIAGMDFVASDKNNRNCPKGVVASLSLGGGYSSSVNSAA
ARLQSSGVMVAVAAGNNNADARNYSPASEPSVCTVGASDRYDRRSSFSNY
GSVLDIFGPGTSILSTWIGGSTRSISGTSMATPHVAGLAAYLMTLGKTTA
ASACRYIADTANKGDLSNIPFGTVNLLAYNNYQA

In embodiments, the enzyme is subtilisin. In embodiments, the enzyme is a naturally occurring form, a homolog, an isoform or a variant of subtilisin that maintains the enzymatic activity of subtilisin (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes subtilisin is as follows:

(SEQ ID NO: 21)
ATGATGAGGAAAAAGAGTTTTTGGCTTGGGATGCTGACGGCCTTCATGCT
CGTGTTCACGATGGCATTCAGCGATTCCGCTTCTGCTGCTCAACCGGCGA
AAAATGTTGAAAAGGATTATATTGTCGGATTTAAGTCAGGAGTGAAAACC
GCATCTGTCAAAAAGGACATCATCAAAGAGAGCGGCGGAAAAGTGGACAA
GCAGTTTAGAATCATCAACGCGGCAAAAGCGAAGCTAGACAAAGAAGCGC
TTAAGGAAGTCAAAAATGATCCGGATGTCGCTTATGTGGAAGAGGATCAT
GTGGCCCATGCCTTGGCGCAAACCGTTCCTTACGGCATTCCTCTCATTAA
AGCGGACAAAGTGCAGGCTCAAGGCTTTAAGGGAGCGAATGTAAAAGTAG
CCGTCCTGGATACAGGAATCCAAGCTTCTCATCCGGACTTGAACGTAGTC
GGCGGAGCAAGCTTTGTGGCTGGCGAAGCTTATAACACCGACGGCAACGG
ACACGGCACACATGTTGCCGGTACAGTAGCTGCGCTTGACAATACAACGG
GTGTATTAGGCGTTGCGCCAAGCGTATCCTTGTACGCGGTTAAAGTACTG
AATTCAAGCGGAAGCGGAACTTACAGCGGCATTGTAAGCGGAATCGAGTG
GGCGACGACAAACGGCATGGATGTTATCAACATGAGTCTTGGAGGACCAT
CAGGCTCAACAGCGATGAAACAGGCGGTTGACAATGCATATGCAAGAGGG
GTTGTCGTTGTGGCGGCTGCTGGGAACAGCGGATCTTCAGGAAACACGAA
TACAATCGGCTATCCTGCGAAATACGACTCTGTCATCGCAGTTGGCGCGG
TAGACTCTAACAGCAACAGAGCTTCATTTTCCAGCGTCGGAGCAGAGCTT
GAAGTCATGGCTCCTGGCGCAGGCGTGTACAGCACTTACCCAACCAGCAC
TTATGCAACATTGAACGGAACGTCAATGGCTTCTCCTCATGTAGCGGGAG
CAGCAGCTTTGATCTTGTCAAAACATCCGAACCTTTCAGCTTCACAAGTC
CGCAACCGTCTCTCCAGTACGGCGACTTATTTGGGAAGCTCCTTCTACTA
TGGAAAAGGTCTGATCAATGTCGAAGCTGCCGCTCAATAA

A non-limiting example of a subtilisin amino acid sequence is as follows:

(SEQ ID NO: 22)
MMRKKSFWLGMLTAFMLVFTMAFSDSASAAQPAKNVEKDYIVGFKSGVKT
ASVKKDIIKESGGKVDKQFRIINAAKAKLDKEALKEVKNDPDVAYVEEDH
VAHALAQTVPYGIPLIKADKVQAQGFKGANVKVAVLDTGIQASHPDLNVV
GGASFVAGEAYNTDGNGHGTHVAGTVAALDNTTGVLGVAPSVSLYAVKVL
NSSGSGSYSGIVSGIEWATTNGMDVINMSLGGASGSTAMKQAVDNAYAKG
VVVVAAAGNSGSSGNTNTIGYPAKYDSVIAVGAVDSNSNRASFSSVGAEL
EVMAPGAGVYSTYPTNTYATLNGTSMASPHVAGAAALILSKHPNLSASQV
RNRLSSTATYLGSSFYYGKGLINVEAAAQ

In embodiments, the enzyme is trypsin. In embodiments, the enzyme is a naturally occurring form, a homolog, an isoform or a variant of trypsin that maintains the enzymatic activity of trypsin (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes trypsin is as follows:

(SEQ ID NO: 23)
ATCGTCGGGGGCTACACCTGCGCAGAGAATTCCGTCCCTTACCAGGTGTC
CCTGAATGCTGGCTACCACTTCTGCGGGGGCTCCCTCATCAATGACCAGT
GGGTGGTGTCCGCGGCTCACTGCTACCAGTACCACATCCAGGTGAGGCTG
GGAGAATACAACATTGATGTCTTGGAGGGTGGTGAGCAGTTCATCGATGC
GTCCAAGATCATCCGCCACCCCAAGTACAGCAGCTGGACTCTGGACAATG
ACATCCTGCTGATCAAACTCTCCACGCCTGCGGTCATCAATGCCCGGGTG
TCCACCTTGCTGCTGCCCAGTGCCTGTGCTTCCGCAGGCACAGAGTGCCT
CATCTCCGGCTGGGGCAACACCCTGAGCAGTGGCGTCAACTACCCGGACC
TGCTGCAATGCCTGGTGGCCCCGCTGCTGAGCCACGCCGACTGTGAAGCC
TCATACCCTGGACAGATCACTAACAACATGATCTGCGCTGGCTTCCTGGA
AGGAGGCAAGGATTCCTGCCAGGGTGACTCTGGCGGCCCTGTGGCTTGCA
ACGGACAGCTCCAGGGCATTGTGTCCTGGGGCTACGGCTGTGCCCAGAAG
GGCAAGCCTGGGGTCTACACCAAGGTCTGCAACTACGTGGACTGGATTCA
GGAGACCATCGCCGCCAAC

A non-limiting example of a trypsin amino acid sequence is as follows:

(SEQ ID NO: 24)
IVGGYTCGANTVPYQVSLNSGYHFCGGSLINSQWVVSAAHCYKSGIQVRL
GEDNINVVEGNEQFISASKSIVHPSYNSNTLNNDIMLIKLKSAASLNSRV
ASISLPTSCASAGTQCLISGWGNTKSSGTSYPDVLKCLKAPILSDSSCKS
AYPGQITSNMFCAGYLEGGKDSCQGDSGGPVVCSGKLQGIVSWGSGCAQK
NKPGVYTKVCNYVSWIKQTIASN

In embodiments, the enzyme is serratiopeptidase. In embodiments, the enzyme is a naturally occurring form, a homolog, an isoform or a variant of serratiopeptidase that maintains the enzymatic activity of serratiopeptidase (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. A non-limiting example of a DNA sequence that encodes serratiopeptidase is as follows:

(SEQ ID NO: 25)
ATGCAATCTACTAAAAAGGCAATTGAAATTACTGAATCCAGCCTCGCTGC
CGCGACAACCGGTTACGATGCTGTAGACGACCTGCTGCATTATCATGAGC
GGGGTAACGGGATTCAGATTAATGGCAAGGATTCATTTTCTAACGAGCAA
GCTGGGCTGTTTATTACCCGTGAGAACCAAACCTGGAACGGTTACAAGGT
ATTTGGCCAGCCGGTCAAATTAACCTTCTCGTTCCCGGACTATAAGTTCT
CTTCCACCAACGTCGCCGGCGACACCGGGCTGAGCAAGTTCAGCGCGGAA
CAGCAGCAGCAGGCTAAGCTGTCGCTGCAGTCCTGGGCCGACGTCGCCAA
TATCACCTTCACCGAAGTGGCGGCCGGTCAAAAGGCCAATATCACCTTCG
GCAACTACAGCCAGGATCGTCCCGGCCACTATGATTACGGCACCCAGGCC
TACGCCTTCCTGCCGAACACCATTTGGCAGGGCCAGGATTTGGGCGGCCA
GACTTGGTACAACGTAAACCAATCCAACGTGAAGCATCCGGCGACCGAAG
ACTACGGCCGCCAGACGTTCACCCATGAGATTGGCCATGCGCTGGGCCTG
AGCCACCCGGGCGACTACAACGCCGGTGAGGGCAACCCGACCTATAGAGA
TGTCACCTATGCGGAAGATACCCGCCAGTTCAGCCTGATGAGCTACTGGA
GTGAAACCAATACCGGTGGCGACAACGGCGGTCACTATGCCGCGGCTCCG
CTGCTGGATGACATTGCCGCCATTCAGCATCTGTATGGCGCCAACCTGTC
GACCCGCACCGGCGACACCGTGTACGGCTTTAACTCCAATACCGGTCGTG
ACTTCCTCAGCACCACCAGCAACTCGCAGAAAGTGATCTTTGCGGCCTGG
GATGCGGGCGGCAACGATACCTTCGACTTCTCCGGTTACACCGCTAACCA
GCGCATCAACCTGAACGAGAAATGGTTCTCCGACGTGGGCGGCCTGAAGG
GCAACGTGTCGATCGCCGCCGGTGTGACCATTGAGAACGCCATTGGCGGT
TCCGGCAACGACGTGATCGTCGGCAACGCGGCCAATAACGTGCTGAAAGG
CGGCGCGGGTAACGACGTGCTGTTCGGCGGCGGCGGGGCGGATGAATTGT
GGGGCGGTGCCGGCAAAGACATCTTCGTGTTCTCTGCCGCCAGCGATTCC
GCACCGGGCGCTTCAGACTGGATCCGCGACTTCCAGAAAGGGATCGACAA
GATCGACCTGTCGTTCTTCAATAAAGAAGCGCAGAGCAGCGATTTCATTC
ACTTCGTCGATCACTTCAGCGGCACGGCCGGTGAGGCGCTGCTGAGCTAC
AACGCGTCCAGCAACGTGACCGATTTGTCGGTGAACATCGGTGGGCATCA
GGCGCCGGACTTCCTGGTGAAAATCGTCGGCCAGGTAGACGTCGCCACGG
ACTTTATCGTGTAA

A non-limiting example of a serratiopeptidase amino acid sequence is as follows:

(SEQ ID NO: 26)
MQSTKKAIEITESSLAAATTGYDAVDDLLHYHERGNGIQINGKDSFSNEQ
AGLFITRENQTWNGYKVFGQPVKLTFSFPDYKFSSTNVAGDTGLSKFSAE
QQQQAKLSLQSWADVANITFTEVAAGQKANITFGNYSQDRPGHYDYGTQA
YAFLPNTIWQGQDLGGQTWYNVNQSNVKHPATEDYGRQTFTHEIGHALGL
SHPGDYNAGEGNPTYRDVTYAEDTRQFSLMSYWSETNTGGDNGGHYAAAP
LLDDIAAIQHLYGANLSTRTGDTVYGFNSNTGRDFLSTTSNSQKVIFAAW
DAGGNDTFDFSGYTANQRINLNEKWFSDVGGLKGNVSIAAGVTIENAIGG
SGNDVIVGNAANNVLKGGAGNDVLFGGGGADELWGGAGKDIFVFSAASDS
APGASDWIRDFQKGIDKIDLSFFNKEAQSSDFIHFVDHFSGTAGEALLSY
NASSNVTDLSVNIGGHQAPDFLVKIVGQVDVATDFIV

In embodiments, the enzyme is a DNAse. In embodiments, the enzyme is a naturally occurring form, a homolog, an isoform or a variant of a DNAse that maintains the enzymatic activity of the DNAse (e.g., within at least 50%, 80%, 90%, 95%, 96%, 97%, 98%, 99% or 100% activity compared to the native protein). In embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the enzyme is a DNAse I. In embodiments, the DNAse I is bovine pancreatic DNAse I. A non-limiting example of a DNA sequence that encodes a bovine pancreatic DNAse I is as follows:

(SEQ ID NO: 27)
TTGAAGATTGCTGCTTTCAACATTAGAACTTTCGGTGAAACTAAAATGTC
TAACGCTACTTTGGCATCTTACATCGTTAGAATTGTCAGAAGATATGATA
TCGTTTTAATTCAAGAAGTTAGAGACTCTCACTTGGTTGCAGTTGGTAAA
TTGTTAGACTACTTGAACCAAGATGACCCAAACACTTACCACTACGTTGT
TTCTGAACCATTGGGTAGAAACTCTTACAAAGAAAGATACTTATTCTTGT
TCAGACCAAACAAAGTTTCAGTTTTGGATACTTACCAATACGACGACGGT
TGCGAATCTTGTGGTAACGATTCTTTCTCCAGAGAACCTGCTGTTGTTAA
ATTCTCATCACACTCTACCAAGGTTAAAGAGTTCGCTATCGTTGCTTTGC
ATTCTGCTCCTTCTGACGCTGTTGCTGAAATTAACTCTTTGTACGACGTT
TACTTAGATGTTCAACAGAAATGGCACTTGAACGACGTCATGTTGATGGG
TGACTTTAACGCTGATTGCTCTTATGTTACTTCTTCTCAATGGTCTTCAA
TTAGATTGAGAACATCTTCAACTTTCCAATGGTTAATTCCTGATTCCGCT
GATACCACTGCTACTAGTACCAACTGTGCTTACGATAGAATCGTTGTTGC
TGGATCATTATTGCAATCTTCTGTTGTCCCAGGTTCAGCGGCCCCTTTCG
ATTTCCAAGCTGCATATGGTTTGTCTAATGAAATGGCTTTAGCCATTTCT
GATCACTACCCAGTTGAAGTCACATTGACATAA

A non-limiting example of a bovine pancreatic DNAse I amino acid sequence is as follows:

(SEQ ID NO: 28)
LKIAAFNIRTFGETKMSNATLASYIVRIVRRYDIVLIQEVRDSHLVAVGK
LLDYLNQDDPNTYHYVVSEPLGRNSYKERYLFLFRPNKVSVLDTYQYDDG
CESCGNDSFSREPAVVKFSSHSTKVKEFAIVALHSAPSDAVAEINSLYDV
YLDVQQKWHLNDVMLMGDFNADCSYVTSSQWSSIRLRTSSTFQWLIPDSA
DTTATSTNCAYDRIVVAGSLLQSSVVPGSAAPFDFQAAYGLSNEMALAIS
DHYPVEVTLT

In embodiments, the composition or combination includes a probiotic bacterium.

In embodiments, the probiotic bacterium is a probiotic a P. sp., Staphylococcus sp., and/or Corynebacterium sp. bacterium.

In embodiments, the probiotic bacterium is a bacterium within the class Betaproteobacteria.

In embodiments, the probiotic bacterium is a probiotic P. acnes bacterium.

In embodiments, the P. acnes bacterium (a) has a 16S ribosomal DNA (rDNA) sequence with a T992C mutation compared to the KPA171202 type strain 16S rDNA sequence set forth as SEQ ID NO: 2; (b) has a 16S rDNA sequence with a T838C mutation compared to the KPA171202 type strain 16S rDNA sequence set forth as SEQ ID NO: 2; (c) has a 16S rDNA sequence with a C1322T mutation compared to the KPA171202 type strain 16S rDNA sequence set forth as SEQ ID NO: 2; (d) has a 16S rDNA sequence with a C986T mutation compared to the KPA171202 type strain 16S rDNA sequence set forth as SEQ ID NO: 2; (e) has a 16S rDNA sequence that is identical to the sequence of SEQ ID NO: 3; (f) has a 16S rDNA sequence that is identical to the sequence of SEQ ID NO: 4; (g) does not comprise a linear plasmid; (h) does not comprise a plasmid that has a virulence factor; and/or (i) does not have a plasmid that encodes an extrachromosomal lipase and/or a tight adhesion virulence factor.

In embodiments, the P. acnes bacterium (a) produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in a planktonic culture; (b) produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in an adherent culture; (c) adheres to epithelial cells at least 50% less than a pathogenic P. acnes strain; and/or (d) is less inflammatory than a pathogenic P. acnes strain.

In embodiments, the combination or composition includes at least one additional probiotic bacterium. In embodiments, the at least one additional probiotic bacterium includes Propionibacterium granulosum and/or Propionibacterium avidum.

In embodiments, a pathogenic P. acnes strain (a) has a 16S rDNA sequence with a G1058C mutation compared to the KPA171202 type strain 16S rDNA sequence set forth as SEQ ID NO: 2; (b) has a 16S rDNA sequence with a G1058C and an A1201C mutation compared to the KPA171202 type strain 16S rDNA sequence set forth as SEQ ID NO: 2; (c) has a 16S rDNA sequence with a G529A mutation compared to the KPA171202 type strain 16S rDNA sequence set forth as SEQ ID NO: 2; (d) has a 16S rDNA sequence with a G1004A and a T1007C mutation compared to the KPA171202 type strain 16S rDNA sequence set forth as SEQ ID NO: 2; (e) has a 16S rDNA sequence with a G1268A mutation compared to the KPA171202 type strain 16S rDNA sequence set forth as SEQ ID NO: 2; (f) has a 16S rDNA sequence with a T554C and a G1058C mutation compared to the KPA171202 type strain 16S rDNA sequence set forth as SEQ ID NO: 2; (g) has a 16S rDNA sequence that is identical to the sequence of SEQ ID NO: 5; (h) has a 16S rDNA sequence that is identical to the sequence of SEQ ID NO: 6; (i) has a 16S rDNA sequence that is identical to the sequence of SEQ ID NO: 7; (j) has a 16S rDNA sequence that is identical to the sequence of SEQ ID NO: 8; (k) has a 16S rDNA sequence that is identical to the sequence of SEQ ID NO: 9; and/or (1) has a 16S rDNA sequence that is identical to the sequence of SEQ ID NO: 10.

In embodiments, the combination or composition further includes at least one additional P. acnes bacteriophage.

In embodiments, the composition or combination includes a pharmaceutically acceptable carrier. In embodiments, the pharmaceutically acceptable carrier includes an emulsion. In embodiments, the emulsion is an oil-in-water emulsion or a water-in-oil emulsion. In embodiments, a combination or combination includes or is in the form of a cream, lotion, suspension, or aqueous solution.

In embodiments, a composition that includes a bacteriophage is provided. In embodiments, the composition is formulated for topical application to the skin (i.e., the composition is a topical composition). In embodiments, the composition is a pharmaceutical composition.

In an aspect, there is provided a pharmaceutical composition including a wild-type P. acnes bacteriophage and an isolated probiotic P. acnes bacterium. In embodiments, the composition further includes a pharmaceutically acceptable carrier.

In an aspect, there is provided a pharmaceutical composition including a bacteriophage and/or an isolated probiotic P. acnes bacterium and a pharmaceutically acceptable carrier.

In embodiments, the pharmaceutical composition is formulated for topical administration to the skin. In embodiments, the pharmaceutically acceptable carrier includes an emulsion. In embodiments, the emulsion is an oil-in-water emulsion or a water-in-oil emulsion. In embodiments, the pharmaceutical composition is in the form of a cream, lotion, suspension, or aqueous solution.

In embodiments, a composition or combination includes at least about 2, 3, 4, 5, 6, 7, 8, 9, or 10 P. acnes bacteriophages. In embodiments, the P. acnes bacteriophages include more than one type of P. acnes bacteriophage.

In embodiments, a combination or composition including an isolated probiotic P. acnes bacterium may further comprise at least one additional bacterium.

In embodiments, a P. acnes bacterium has a 16S rDNA sequence that includes a T992C, T838C, C1322T, and/or a C986T mutation compared to the KPA171202 type strain 16S rDNA sequence set forth as SEQ ID NO: 2. In embodiments, the P. acnes bacterium includes a 16S rDNA sequence with a T838C and a C1322T mutation compared to the KPA171202 type strain 16S rDNA sequence set forth as SEQ ID NO: 2. In embodiments, the P. acnes bacterium is the ProI strain. In embodiments, the P. acnes bacterium includes a 16S rDNA sequence with a C986T and a T992C mutation compared to the KPA171202 type strain 16S rDNA sequence set forth as SEQ ID NO: 2. In embodiments, the P. acnes bacterium is the ProII strain. In embodiments, the P. acnes bacterium: (a) includes a 16S rDNA sequence with a T992C mutation compared to the KPA171202 type strain 16S rDNA sequence set forth as SEQ ID NO: 2; (b) includes a 16S rDNA sequence with a T838C mutation compared to the KPA171202 type strain 16S rDNA sequence set forth as SEQ ID NO: 2; (c) includes a 16S rDNA sequence with a C1322T mutation compared to the KPA171202 type strain 16S rDNA sequence set forth as SEQ ID NO: 2; (d) includes a 16S rDNA sequence with a C986T mutation compared to the KPA171202 type strain 16S rDNA sequence set forth as SEQ ID NO: 2; (e) includes a 16S rDNA sequence that is identical to the sequence of SEQ ID NO: 3; (f) includes a 16S rDNA sequence that is identical to the sequence of SEQ ID NO: 4; (g) does not comprise a linear plasmid; (h) does not include a plasmid that includes a virulence factor; and/or (i) does not include a plasmid that encodes an extrachromosomal lipase and/or a tight adhesion virulence factor. In embodiments, the P. acnes bacterium has a 16S rDNA sequence that is identical to the sequence of SEQ ID NO: 3 or 4.

In embodiments, the P. acnes bacterium: (a) produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in a planktonic culture; (b) produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in an adherent culture; (c) adheres to epithelial cells at least 50% less than a pathogenic P. acnes strain; and/or (d) is less inflammatory than a pathogenic P. acnes strain. In embodiments, the pathogenic P. acnes strain (a) has a 16S rDNA sequence with a G1058C mutation compared to the KPA171202 type strain 16S rDNA sequence set forth as SEQ ID NO: 2; (b) has a 16S rDNA sequence with a G1058C and an A1201C mutation compared to the KPA171202 type strain 16S rDNA sequence set forth as SEQ ID NO: 2; (c) has a 16S rDNA sequence with a G529A mutation compared to the KPA171202 type strain 16S rDNA sequence set forth as SEQ ID NO: 2; (d) has a 16S rDNA sequence with a G1004A and a T1007C mutation compared to the KPA171202 type strain 16S rDNA sequence set forth as SEQ ID NO: 2; (e) has a 16S rDNA sequence with a G1268A mutation compared to the KPA171202 type strain 16S rDNA sequence set forth as SEQ ID NO: 2; (f) has a 16S rDNA sequence with a T554C and a G1058C mutation compared to the KPA171202 type strain 16S rDNA sequence set forth as SEQ ID NO: 2; (g) has a 16S rDNA sequence that is identical to the sequence of SEQ ID NO: 5; (h) has a 16S rDNA sequence that is identical to the sequence of SEQ ID NO: 6; (i) has a 16S rDNA sequence that is identical to the sequence of SEQ ID NO: 7; (j) has a 16S rDNA sequence that is identical to the sequence of SEQ ID NO: 8; (k) has a 16S rDNA sequence that is identical to the sequence of SEQ ID NO: 9; and/or (1) has a 16S rDNA sequence that is identical to the sequence of SEQ ID NO: 10.

| SEQ ID NO: 2 is the 16S rDNA sequence for the KPA171202 type strain, | |
|---|---|
| and is as follows: | |
| 1 | TTTTTCATTG GAGAGTTTGA TCCTGGCTCA GGACGAACGC TGGCGGCGTG CTTAACACAT |
| 61 | GCAAGTCGAA CGGAAAGGCC CTGCTTTTGT GGGGTGCTCG AGTGGCGAAC GGGTGAGTAA |
| 121 | CACGTGAGTA ACCTGCCCTT GACTTTGGGA TAACTTCAGG AAACTGGGGC TAATACCGGA |
| 181 | TAGGAGCTCC TGCTGCATGG TGGGGGTTGG AAAGTTTCGG CGGTTGGGGA TGGACTCGCG |
| 241 | GCTTATCAGC TTGTTGGTGG GGTAGTGGCT TACCAAGGCT TTGACGGGTA GCCGGCCTGA |
| 301 | GAGGGTGACC GGCCACATTG GGACTGAGAT ACGGCCCAGA CTCCTACGGG AGGCAGCAGT |
| 361 | GGGGAATATT GCACAATGGG CGGAAGCCTG ATGCAGCAAC GCCGCGTGCG GGATGACGGC |
| 421 | CTTCGGGTTG TAAACCGCTT TCGCCTGTGA CGAAGCGTGA GTGACGGTAA TGGGTAAAGA |
| 481 | AGCACCGGCT AACTACGTGC CAGCAGCCGC GGTGATACGT AGGGTGCGAG CGTTGTCCGG |
| 541 | ATTTATTGGG CGTAAAGGGC TCGTAGGTGG TTGATCGCGT CGGAAGTGTA ATCTTGGGGC |
| 601 | TTAACCCTGA GCGTGCTTTC GATACGGGTT GACTTGAGGA AGGTAGGGGA GAATGGAATT |
| 661 | CCTGGTGGAG CGGTGGAATG CGCAGATATC AGGAGGAACA CCAGTGGCGA AGGCGGTTCT |
| 721 | CTGGGCCTTT CCTGACGCTG AGGAGCGAAA GCGTGGGGAG CGAACAGGCT TAGATACCCT |
| 781 | GGTAGTCCAC GCTGTAAACG GTGGGTACTA GGTGTGGGGT CCATTCCACG GGTTCCGTGC |
| 841 | CGTAGCTAAC GCTTTAAGTA CCCCGCCTGG GGAGTACGGC CGCAAGGCTA AAACTCAAAG |
| 901 | GAATTGACGG GGCCCCGCAC AAGCGGCGGA GCATGCGGAT TAATTCGATG CAACGCGTAG |
| 961 | AACCTTACCT GGGTTTGACA TGGATCGGGA GTGCTCAGAG ATGGGTGTGC CTCTTTTGGG |
| 1021 | GTCGGTTCAC AGGTGGTGCA TGGCTGTCGT CAGCTCGTGT CGTGAGATGT TGGGTTAAGT |
| 1081 | CCCGCAACGA GCGCAACCCT TGTTCACTGT TGCCAGCACG TTATGGTGGG GACTCAGTGG |
| 1141 | AGACCGCCGG GGTCAACTCG GAGGAAGGTG GGGATGACGT CAAGTCATCA TGCCCCTTAT |
| 1201 | GTCCAGGGCT TCACGCATGC TACAATGGCT GGTACAGAGA GTGGCGAGCC TGTGAGGGTG |
| 1261 | AGCGAATCTC GGAAAGCCGG TCTCAGTTCG GATTGGGGTC TGCAACTCGA CCTCATGAAG |
| 1321 | TCGGAGTCGC TAGTAATCGC AGATCAGCAA CGCTGCGGTG AATACGTTCC CGGGGCTTGT |
| 1381 | ACACACCGCC CGTCAAGTCA TGAAAGTTGG TAACACCCGA AGCCGGTGGC CTAACCGTTG |
| 1441 | TGGGGGAGCC GTCGAAGGTG GGACTGGTGA TTAGGACTAA GTCGTAACAA GGTAGCCGTA |
| 1501 | CCGGAAGGTG CGGCTGGATC ACCTCCTTTC TAAGGAG |
| SEQ ID NO: 3 is the 16S rDNA sequence for the ProI probiotic strain, | |
| and is as follows: | |
| Nucleotides 838 . . . 838 | |
| ProI Mutation T838C | |
| Nucleotides 1322 . . . 1322 | |
| ProI Mutation C1322T | |
| 1 | TTTTTCATTG GAGAGTTTGA TCCTGGCTCA GGACGAACGC TGGCGGCGTG CTTAACACAT |
| 61 | GCAAGTCGAA CGGAAAGGCC CTGCTTTTGT GGGGTGCTCG AGTGGCGAAC GGGTGAGTAA |
| 121 | CACGTGAGTA ACCTGCCCTT GACTTTGGGA TAACTTCAGG AAACTGGGGC TAATACCGGA |
| 181 | TAGGAGCTCC TGCTGCATGG TGGGGGTTGG AAAGTTTCGG CGGTTGGGGA TGGACTCGCG |
| 241 | GCTTATCAGC TTGTTGGTGG GGTAGTGGCT TACCAAGGCT TTGACGGGTA GCCGGCCTGA |
| 301 | GAGGGTGACC GGCCACATTG GGACTGAGAT ACGGCCCAGA CTCCTACGGG AGGCAGCAGT |
| 361 | GGGGAATATT GCACAATGGG CGGAAGCCTG ATGCAGCAAC GCCGCGTGCG GGATGACGGC |
| 421 | CTTCGGGTTG TAAACCGCTT TCGCCTGTGA CGAAGCGTGA GTGACGGTAA TGGGTAAAGA |
| 481 | AGCACCGGCT AACTACGTGC CAGCAGCCGC GGTGATACGT AGGGTGCGAG CGTTGTCCGG |
| 541 | ATTTATTGGG CGTAAAGGGC TCGTAGGTGG TTGATCGCGT CGGAAGTGTA ATCTTGGGGC |
| 601 | TTAACCCTGA GCGTGCTTTC GATACGGGTT GACTTGAGGA AGGTAGGGGA GAATGGAATT |
| 661 | CCTGGTGGAG CGGTGGAATG CGCAGATATC AGGAGGAACA CCAGTGGCGA AGGCGGTTCT |
| 721 | CTGGGCCTTT CCTGACGCTG AGGAGCGAAA GCGTGGGGAG CGAACAGGCT TAGATACCCT |
| 781 | GGTAGTCCAC GCTGTAAACG GTGGGTACTA GGTGTGGGGT CCATTCCACG GGTTCCGCGC |
| 841 | CGTAGCTAAC GCTTTAAGTA CCCCGCCTGG GGAGTACGGC CGCAAGGCTA AAACTCAAAG |
| 901 | GAATTGACGG GGCCCCGCAC AAGCGGCGGA GCATGCGGAT TAATTCGATG CAACGCGTAG |
| 961 | AACCTTACCT GGGTTTGACA TGGATCGGGA GTGCTCAGAG ATGGGTGTGC CTCTTTTGGG |
| 1021 | GTCGGTTCAC AGGTGGTGCA TGGCTGTCGT CAGCTCGTGT CGTGAGATGT TGGGTTAAGT |
| 1081 | CCCGCAACGA GCGCAACCCT TGTTCACTGT TGCCAGCACG TTATGGTGGG GACTCAGTGG |
| 1141 | AGACCGCCGG GGTCAACTCG GAGGAAGGTG GGGATGACGT CAAGTCATCA TGCCCCTTAT |
| 1201 | GTCCAGGGCT TCACGCATGC TACAATGGCT GGTACAGAGA GTGGCGAGCC TGTGAGGGTG |
| 1261 | AGCGAATCTC GGAAAGCCGG TCTCAGTTCG GATTGGGGTC TGCAACTCGA CCTCATGAAG |
| 1321 | TTGGAGTCGC TAGTAATCGC AGATCAGCAA CGCTGCGGTG AATACGTTCC CGGGGCTTGT |
| 1381 | ACACACCGCC CGTCAAGTCA TGAAAGTTGG TAACACCCGA AGCCGGTGGC CTAACCGTTG |
| 1441 | TGGGGGAGCC GTCGAAGGTG GGACTGGTGA TTAGGACTAA GTCGTAACAA GGTAGCCGTA |
| 1501 | CCGGAAGGTG CGGCTGGATC ACCTCCTTTC TAAGGAG |
| SEQ ID NO: 4 is the 16S rDNA sequence for the ProII probiotic strain, | |
| and is as follows: | |
| Nucleotides 986 . . . 986 | |
| ProII Mutation C986T | |
| Nucleotides 992 . . . 992 | |
| ProII Mutation T992C | |
| 1 | TTTTTCATTG GAGAGTTTGA TCCTGGCTCA GGACGAACGC TGGCGGCGTG CTTAACACAT |
| 61 | GCAAGTCGAA CGGAAAGGCC CTGCTTTTGT GGGGTGCTCG AGTGGCGAAC GGGTGAGTAA |
| 121 | CACGTGAGTA ACCTGCCCTT GACTTTGGGA TAACTTCAGG AAACTGGGGC TAATACCGGA |
| 181 | TAGGAGCTCC TGCTGCATGG TGGGGGTTGG AAAGTTTCGG CGGTTGGGGA TGGACTCGCG |
| 241 | GCTTATCAGC TTGTTGGTGG GGTAGTGGCT TACCAAGGCT TTGACGGGTA GCCGGCCTGA |
| 301 | GAGGGTGACC GGCCACATTG GGACTGAGAT ACGGCCCAGA CTCCTACGGG AGGCAGCAGT |
| 361 | GGGGAATATT GCACAATGGG CGGAAGCCTG ATGCAGCAAC GCCGCGTGCG GGATGACGGC |
| 421 | CTTCGGGTTG TAAACCGCTT TCGCCTGTGA CGAAGCGTGA GTGACGGTAA TGGGTAAAGA |
| 481 | AGCACCGGCT AACTACGTGC CAGCAGCCGC GGTGATACGT AGGGTGCGAG CGTTGTCCGG |
| 541 | ATTTATTGGG CGTAAAGGGC TCGTAGGTGG TTGATCGCGT CGGAAGTGTA ATCTTGGGGC |
| 601 | TTAACCCTGA GCGTGCTTTC GATACGGGTT GACTTGAGGA AGGTAGGGGA GAATGGAATT |
| 661 | CCTGGTGGAG CGGTGGAATG CGCAGATATC AGGAGGAACA CCAGTGGCGA AGGCGGTTCT |
| 721 | CTGGGCCTTT CCTGACGCTG AGGAGCGAAA GCGTGGGGAG CGAACAGGCT TAGATACCCT |
| 781 | GGTAGTCCAC GCTGTAAACG GTGGGTACTA GGTGTGGGGT CCATTCCACG GGTTCCGTGC |
| 841 | CGTAGCTAAC GCTTTAAGTA CCCCGCCTGG GGAGTACGGC CGCAAGGCTA AAACTCAAAG |
| 901 | GAATTGACGG GGCCCCGCAC AAGCGGCGGA GCATGCGGAT TAATTCGATG CAACGCGTAG |
| 961 | AACCTTACCT GGGTTTGACA TGGATTGGGA GCGCTCAGAG ATGGGTGTGC CTCTTTTGGG |
| 1021 | GTCGGTTCAC AGGTGGTGCA TGGCTGTCGT CAGCTCGTGT CGTGAGATGT TGGGTTAAGT |
| 1081 | CCCGCAACGA GCGCAACCCT TGTTCACTGT TGCCAGCACG TTATGGTGGG GACTCAGTGG |
| 1141 | AGACCGCCGG GGTCAACTCG GAGGAAGGTG GGGATGACGT CAAGTCATCA TGCCCCTTAT |
| 1201 | GTCCAGGGCT TCACGCATGC TACAATGGCT GGTACAGAGA GTGGCGAGCC TGTGAGGGTG |
| 1261 | AGCGAATCTC GGAAAGCCGG TCTCAGTTCG GATTGGGGTC TGCAACTCGA CCTCATGAAG |
| 1321 | TCGGAGTCGC TAGTAATCGC AGATCAGCAA CGCTGCGGTG AATACGTTCC CGGGGCTTGT |
| 1381 | ACACACCGCC CGTCAAGTCA TGAAAGTTGG TAACACCCGA AGCCGGTGGC CTAACCGTTG |
| 1441 | TGGGGGAGCC GTCGAAGGTG GGACTGGTGA TTAGGACTAA GTCGTAACAA GGTAGCCGTA |
| 1501 | CCGGAAGGTG CGGCTGGATC ACCTCCTTTC TAAGGAG |

In embodiments, the P. acnes bacterium produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in a planktonic culture. In embodiments, the P. acnes bacterium produces about 1-5%, 1-10%, 1-20%, 1-30%, 5-50%, 5-40%, 5-30%, 5-20%, 5-10%, 10-50%, 10-40%, 10-30%, 10-20%, 20-50%, 20-40%, or 20-30% of the level of lipase that is produced by a pathogenic P. acnes strain when grown in a planktonic culture. In embodiments, the P. acnes bacterium produces less than about 5% of the level of lipase that is produced by a pathogenic P. acnes strain when grown in a planktonic culture. In embodiments, the P. acnes bacterium produces less than about 10% of the level of lipase that is produced by a pathogenic P. acnes strain when grown in a planktonic culture. In embodiments, the P. acnes bacterium produces less than about 20% of the level of lipase that is produced by a pathogenic P. acnes strain when grown in a planktonic culture. In embodiments, the P. acnes bacterium produces less than about 30% of the level of lipase that is produced by a pathogenic P. acnes strain when grown in a planktonic culture. In embodiments, the P. acnes bacterium produces less than about 40% of the level of lipase that is produced by a pathogenic P. acnes strain when grown in a planktonic culture. In embodiments, the P. acnes bacterium produces less than about 50% of the level of lipase that is produced by a pathogenic P. acnes strain when grown in a planktonic culture. In embodiments, the P. acnes bacterium produces less than about 1%, 5%, 10%, 15%, 20%, 25%, 30%, 35%, 40%, 45%, 50%, 55%, 60%, 65%, 70%, 75%, 80%, 85%, 90% or 95% of the level of lipase that is produced by a pathogenic P. acnes strain when grown in an adherent culture. In embodiments, the P. acnes bacterium produces less than about 5% of the level of lipase that is produced by a pathogenic P. acnes strain when grown in an adherent culture. In embodiments, the P. acnes bacterium produces less than about 10% of the level of lipase that is produced by a pathogenic P. acnes strain when grown in an adherent culture. In embodiments, the P. acnes bacterium produces less than about 20% of the level of lipase that is produced by a pathogenic P. acnes strain when grown in an adherent culture. In embodiments, the P. acnes bacterium produces less than about 30% of the level of lipase that is produced by a pathogenic P. acnes strain when grown in an adherent culture. In embodiments, the P. acnes bacterium produces less than about 40% of the level of lipase that is produced by a pathogenic P. acnes strain when grown in an adherent culture. In embodiments, the P. acnes bacterium produces less than about 50% of the level of lipase that is produced by a pathogenic P. acnes strain when grown in an adherent culture. In embodiments, the P. acnes bacterium produces about 1-5%, 1-10%, 1-20%, 1-30%, 5-50%, 5-40%, 5-30%, 5-20%, 5-10%, 10-50%, 10-40%, 10-30%, 10-20%, 20-50%, 20-40%, or 20-30% of the level of lipase that is produced by a pathogenic P. acnes strain when grown in an adherent culture. In embodiments, the lipase is extracellular lipase.

In embodiments, the level of lipase produced by a P. acnes bacterium (e.g., a probiotic or a pathogenic P. acnes bacterium, such as for comparison) is the level of lipase in culture supernatant. In embodiments, the culture supernatant is filtered. In embodiments, the culture supernatant is from a liquid (planktonic) culture. In embodiments, the culture supernatant is from an adherent culture. Non-limiting examples of methods for detecting a level of lipase include absorbance, Bradford protein assays, Biuret test derived assays, fluorescamine, amino black, colloidal gold, nitrogen detection, High-performance liquid chromatography (HPLC), Liquid chromatography-mass spectrometry (LC/MS), enzyme-linked immunosorbent assay (ELISA), protein immunoprecipitation, immunoelectrophoresis, and Western blot.

In embodiments, the P. acnes bacterium adheres to epithelial cells at least about 1%, 5%, 10%, 15%, 20%, 25%, 30%, 35%, 40%, 45%, 50%, 55%, 60%, 65%, 70%, 75%, 80%, 85%, 90% or 95% less than a pathogenic P. acnes strain. In embodiments, the P. acnes bacterium adheres to epithelial cells at least about 50% less than a pathogenic P. acnes strain. In embodiments, the P. acnes bacterium adheres to epithelial cells at least about 60% less than a pathogenic P. acnes strain. In embodiments, the P. acnes bacterium adheres to epithelial cells at least about 70% less than a pathogenic P. acnes strain. In embodiments, the P. acnes bacterium adheres to epithelial cells at least about 80% less than a pathogenic P. acnes strain. In embodiments, the P. acnes bacterium adheres to epithelial cells at least about 90% less than a pathogenic P. acnes strain. In embodiments, the P. acnes bacterium adheres to epithelial cells 1-5%, 1-10%, 1-20%, 1-30%, 5-50%, 5-40%, 5-30%, 5-20%, 5-10%, 10-50%, 10-40%, 10-30%, 10-20%, 20-50%, 20-40%, 20-30%, 50-60, 50-70, 50-80, 50-90, 60-80, 70-90 less than a pathogenic P. acnes strain.

In embodiments, adherence of a P. acnes bacterium (e.g., a probiotic or a pathogenic P. acnes bacterium, such as for comparison) to epithelial cells is determined using A-432 epithelial cells. In embodiments, the epithelial cells are confluent on a tissue culture plate or flask. In embodiments, adherence is detected by determining a number of colonies that are formed by P. acnes bacteria that have adhered to cultured epithelial cells.

In embodiments, the P. acnes bacterium is less inflammatory than a pathogenic P. acnes strain.

In embodiments, a P. acnes bacterium is less inflammatory than a pathogenic P. acnes strain if a lower level of an inflammatory cytokine (e.g., at least about 10%, 20%, 30%, 40%, 50%, 60%, 70%, 80%, or 90% less) is released by an immune cell that contacts the P. acnes bacterium or a compound produced by the P. acnes bacterium compared to a bacterium of the pathogenic P. acnes strain or a compound produced by the bacterium of the pathogenic P. acnes strain. In embodiments, a P. acnes bacterium is less inflammatory than a pathogenic P. acnes strain if a lower level of an inflammatory cytokine is released in tissue (such as skin tissue) that is contacted with P. acnes bacterium. In embodiments, the tissue is skin tissue. In embodiments, the tissue is ear tissue, e.g., of a mouse. In embodiments, the inflammatory cytokine is IL-10, IL-6, IL-17, or TNFα, or any combination thereof.

In embodiments, the pathogenic P. acnes strain (a) has a 16S rDNA sequence with a G1058C mutation compared to the KPA171202 type strain 16S rDNA sequence set forth as SEQ ID NO: 2; (b) has a 16S rDNA sequence with a G1058C and an A1201C mutation compared to the KPA171202 type strain 16S rDNA sequence set forth as SEQ ID NO: 2; (c) has a 16S rDNA sequence with a G529A mutation compared to the KPA171202 type strain 16S rDNA sequence set forth as SEQ ID NO: 2; (d) has a 16S rDNA sequence with a G1004A and a T1007C mutation compared to the KPA171202 type strain 16S rDNA sequence set forth as SEQ ID NO: 2; (e) has a 16S rDNA sequence with a G1268A mutation compared to the KPA171202 type strain 16S rDNA sequence set forth as SEQ ID NO: 2; (f) has a 16S rDNA sequence with a T554C and a G1058C mutation compared to the KPA171202 type strain 16S rDNA sequence set forth as SEQ ID NO: 2; (g) has a 16S rDNA sequence that is identical to the sequence of SEQ ID NO: 5; (h) has a 16S rDNA sequence that is identical to the sequence of SEQ ID NO: 6; (i) has a 16S rDNA sequence that is identical to the sequence of SEQ ID NO: 7; (j) has a 16S rDNA sequence that is identical to the sequence of SEQ ID NO: 8; (k) has a 16S rDNA sequence that is identical to the sequence of SEQ ID NO: 9; and/or (1) has a 16S rDNA sequence that is identical to the sequence of SEQ ID NO: 10.

SEQ ID NO: 5 is as follows (mutations compared to the 16S
sequence of the type strain KPA171202 are underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTGATAC
GTAGGGTGCGAGCGTTGTCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGGAGTGCTCAGAGATGGGTGTGCCTCTTTTGGGGTCGGT
TCACAGGTGGTGCATGCCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCCTCATGCCCC
TTATGTCCAGGGCTTCACGCATGCTACAATGGCTGGTACAGAGAGTGGCGAGCCTGT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA
SEQ ID NO: 6 is as follows (a mutation compared to the 16S
sequence of the type strain KPA171202 is underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTGATAC
GTAGGGTGCGAGCGTTGTCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGGAGTGCTCAGAGATGGGTGTGCCTCTTTTGGGGTCGGT
TCACAGGTGGTGCATGCCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCATCATGCCCC
TTATGTCCAGGGCTTCACGCATGCTACAATGGCTGGTACAGAGAGTGGCGAGCCTGT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA
SEQ ID NO: 7 is as follows (a mutation compared to the 16S
sequence of the type strain KPA171202 is underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTAATAC
GTAGGGTGCGAGCGTTGTCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGGAGTGCTCAGAGATGGGTGTGCCTCTTTTGGGGTCGGT
TCACAGGTGGTGCATGGCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCATCATGCCCC
TTATGTCCAGGGCTTCACGCATGCTACAATGGCTGGTACAGAGAGTGGCGAGCCTGT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA
SEQ ID NO: 8 is as follows (mutations compared to the 16S
sequence of the type strain KPA171202 are underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTGATAC
GTAGGGTGCGAGCGTTGTCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGAAGCGCTCAGAGATGGGTGTGCCTCTTTTGGGGTCGGT
TCACAGGTGGTGCATGGCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCATCATGCCCC
TTATGTCCAGGGCTTCACGCATGCTACAATGGCTGGTACAGAGAGTGGCGAGCCTGT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA
SEQ ID NO: 9 is as follows (a mutation compared to the 16S
sequence of the type strain KPA171202 is underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTGATAC
GTAGGGTGCGAGCGTTGTCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGGAGTGCTCAGAGATGGGTGTGCCTCTTTTGGGGTCGGT
TCACAGGTGGTGCATGGCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCATCATGCCCC
TTATGTCCAGGGCTTCACGCATGCTACAATGGCTGGTACAGAGAGTGGCGAGCCTAT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA
SEQ ID NO: 10 is as follows (mutations compared to the 16S
sequence of the type strain KPA171202 are underlined):
AGAGTTTGATCCTGGCTCAGGACGAACGCTGGCGGCGTGCTTAACACATGCAAGTC
GAACGGAAAGGCCCTGCTTTTGTGGGGTGCTCGAGTGGCGAACGGGTGAGTAACAC
GTGAGTAACCTGCCCTTGACTTTGGGATAACTTCAGGAAACTGGGGCTAATACCGGA
TAGGAGCTCCTGCTGCATGGTGGGGGTTGGAAAGTTTCGGCGGTTGGGGATGGACT
CGCGGCTTATCAGCTTGTTGGTGGGGTAGTGGCTTACCAAGGCTTTGACGGGTAGCC
GGCCTGAGAGGGTGACCGGCCACATTGGGACTGAGATACGGCCCAGACTCCTACGG
GAGGCAGCAGTGGGGAATATTGCACAATGGGCGGAAGCCTGATGCAGCAACGCCGC
GTGCGGGATGACGGCCTTCGGGTTGTAAACCGCTTTCGCCTGTGACGAAGCGTGAGT
GACGGTAATGGGTAAAGAAGCACCGGCTAACTACGTGCCAGCAGCCGCGGTGATAC
GTAGGGTGCGAGCGTTGCCCGGATTTATTGGGCGTAAAGGGCTCGTAGGTGGTTGAT
CGCGTCGGAAGTGTAATCTTGGGGCTTAACCCTGAGCGTGCTTTCGATACGGGTTGA
CTTGAGGAAGGTAGGGGAGAATGGAATTCCTGGTGGAGCGGTGGAATGCGCAGATA
TCAGGAGGAACACCAGTGGCGAAGGCGGTTCTCTGGGCCTTTCCTGACGCTGAGGA
GCGAAAGCGTGGGGAGCGAACAGGCTTAGATACCCTGGTAGTCCACGCTGTAAACG
GTGGGTACTAGGTGTGGGGTCCATTCCACGGGTTCCGTGCCGTAGCTAACGCTTTAA
GTACCCCGCCTGGGGAGTACGGCCGCAAGGCTAAAACTCAAAGGAATTGACGGGGC
CCCGCACAAGCGGCGGAGCATGCGGATTAATTCGATGCAACGCGTAGAACCTTACC
TGGGTTTGACATGGATCGGGAGTGCTCAGAGATGGGTGTGCCTCTTTTGGGGTCGGT
TCACAGGTGGTGCATGCCTGTCGTCAGCTCGTGTCGTGAGATGTTGGGTTAAGTCCC
GCAACGAGCGCAACCCTTGTTCACTGTTGCCAGCACGTTATGGTGGGGACTCAGTGG
AGACCGCCGGGGTCAACTCGGAGGAAGGTGGGGATGACGTCAAGTCATCATGCCCC
TTATGTCCAGGGCTTCACGCATGCTACAATGGCTGGTACAGAGAGTGGCGAGCCTGT
GAGGGTGAGCGAATCTCGGAAAGCCGGTCTCAGTTCGGATTGGGGTCTGCAACTCG
ACCTCATGAAGTCGGAGTCGCTAGTAATCGCAGATCAGCAACGCTGCGGTGAATAC
GTTCCCGGGGCTTGTACACACCGCCCGTCAAGTCATGAAAGTTGGTAACACCCGAA
GCCGGTGGCCTAACCGTTGTGGGGGAGCCGTCGAAGGTGGGACTGGTGATTAGGAC
TAAGTCGTAACAAGGTAGCCGTACCGGAAGGTGCGGCTGGATCACCTCCTTTCTAAG
GA

In embodiments, the at least one additional bacterium comprises, consists essentially of, or consists of a probiotic bacterium. In embodiments, the at least one bacterium includes 1, 2, 3, 4, 5, 6, 7, 8, 9, or 10 bacterial strains and/or species, less than about 10, 9, 8, 7, 6, 5, 4, 3, or 2 bacterial strains and/or species, or 1-10, 2-10, 3-10, 4-10, 5-10, 1-5, 2-5, 3-5, or 4-5 bacterial strains and/or species. In embodiments, the at least one bacterium includes a plurality of bacterial strains and/or species, e.g., at least about 2, 3, 4, 5, 6, 7, 8, 9, 10 bacterial strains and/or species. In embodiments, the least one bacterium includes an isolated Propionibacterium granulosum bacterium, an isolated Propionibacterium avidum bacterium, an isolated Staphylococcus epidermidis bacterium, an isolated Staphylococcus aureus bacterium, and/or an isolated Corynebacterium jeikeium bacterium. In embodiments, the least one bacterium includes 1, 2 (of any combination of), 3 (of any combination of), 4 (of any combination of), or 5 of an isolated Propionibacterium granulosum bacterium, an isolated Propionibacterium avidum bacterium, an isolated Staphylococcus epidermidis bacterium, an isolated Staphylococcus aureus bacterium, and/or an isolated Corynebacterium jeikeium bacterium.

In embodiments, a composition or combination provided herein includes an enhancing peptide or enzyme. In embodiments, the enhancing peptide or enzyme has one or more or any combination of the following properties: biofilm degradation, improving skin penetration, antibacterial, reducing inflammation (e.g., of the skin), reducing irritation (e.g., of the skin), reducing redness (e.g., of the skin), firming skin, removing lines, removing wrinkles, or otherwise improving appearance (e.g., of the skin).

In an aspect, a composition that includes a P. acnes bacteriophage and an anti-acne compound is provided. In embodiments, the composition includes a pharmaceutically acceptable carrier. In embodiments, the dose of the P. acnes bacteriophage is adjusted (e.g., increased or decreased) for stability. In embodiments, the dose of the P. acnes bacteriophage is adjusted up or down depending on the anti-acne compound to adjust for its stability in combination with the anti-acne compound.

In an aspect, a combination or system that includes a P. acnes bacteriophage and one or more anti-acne compounds is provided. In an example, the bacteriophage is within one composition (e.g., within one vessel such as a bottle, tube, or other container), and the one or more anti-acne compounds are in a separate composition (within another vessel such as a bottle, tube, or other container). In embodiments, the composition that includes the bacteriophage includes a pharmaceutically acceptable carrier. In embodiments, the composition that includes the anti-acne compound includes a pharmaceutically acceptable carrier. In embodiments, an additional one or more compounds (e.g. an enzyme, a hydrating compound, an ultraviolet radiation absorbing or blocking compound, etc.) are present in the composition that includes the bacteriophage, the composition that includes the one or more anti-acne compounds, or a third separate composition (within a third vessel such as a bottle, tube, or other container). In embodiments, one or more probiotic bacteria are present in the composition that includes the bacteriophage, the composition that includes the one or more anti-acne compounds, or a third separate composition (within a third vessel such as a bottle, tube, or other container). In embodiments, the combination or system further includes instructions for administration. In embodiments, the combination or system includes at least about 2, 3, 4, 5, 6, 7, 8, 9, or 10 P. acnes bacteriophages.

In an aspect, a combination or system that includes a P. acnes bacteriophage and one or more probiotic bacteria and/or one or more compounds (such as one or more enzymes or anti-acne compounds) is provided. In an example, the bacteriophage is within one composition (e.g., within one vessel such as a bottle, tube, or other container), and the one or more probiotic bacteria are in a separate composition (within another vessel such as a bottle, tube, or other container), and optionally, an additional one or more compounds are present in the composition that includes the bacteriophage, the composition that includes the one or more probiotic bacteria, or a third separate composition (within a third vessel such as a bottle, tube, or other container). In embodiments, the combination or system further includes instructions for administration. In embodiments, the combination or system includes at least about 2, 3, 4, 5, 6, 7, 8, 9, or 10 P. acnes bacteriophages.

In embodiments, a system, combination, or composition includes an enzyme such as a biofilm degradation enzyme or an anti-aging enzyme. Non-limiting examples of biofilm degradation enzymes include DNAses (e.g., DNAse I), proteases (e.g., papain, bromelain, Trypsin, Proteinase K, Subtilisin, or serratiopeptidase), glycosidases (e.g., dispersin, alginate lyase, amylase, or cellulase). Non-limiting examples of anti-aging enzymes include superoxide dismutase, and peroxidase.

In embodiments, a system, combination, or composition includes a topical retinoid, an antibiotic, and/or an alpha-hydroxy acid. In embodiments, a system or composition further includes a topical retinoid. In embodiments, a system or composition further includes an antibiotic. In embodiments, a system or composition further includes an alpha-hydroxy acid. In embodiments, the system or composition further includes benzoyl peroxide, salicylic acid, sulfur, resorcinol, resorcinol monoacetate, or any combination thereof. In embodiments, the benzoyl peroxide is present at a concentration of 2.5% to 10%, e.g., about 2.5%, 3%, 3.5%, 4%, 4.5%, 5%, 5.5%, 6%, 6.5%, 7%, 7.5%, 8%, 8.5%, 9%, 9.5%, or 10% (weight/volume). In embodiments, the benzoyl peroxide is present at a concentration of less than 2.5% but greater than about 0.1%, 0.5%, 1%, 1.5%, or 2% (weight/volume). In embodiments, the salicylic acid is present at a concentration of 0.5% to 2%, e.g., about 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% (weight/volume). In embodiments, the salicylic acid is present at a concentration of less than 0.5% but greater than about 0.1% (weight/volume). In embodiments, the sulfur is present at a concentration of 3% to 10%, e.g., about 3%, 3.5%, 4%, 4.5%, 5%, 5.5%, 6%, 6.5%, 7%, 7.5%, 8%, 8.5%, 9%, 9.5%, or 10% (weight/volume). In embodiments, the sulfur is present at a concentration of less than 3% but greater than about 0.1%, 0.5%, 1, 1.5%, 2%, or 2.5% (weight/volume). In embodiments, resorcinol is present at a concentration of 2% and sulfur is present at a concentration of 3% to 8% (e.g., about 2.5%, 3%, 3.5%, 4%, 4.5%, 5%, 5.5%, 6%, 6.5%, 7%, 7.5%, or 8%) (weight/volume). In embodiments, resorcinol monoacetate is present at a concentration of 3% and sulfur is present at a concentration of 3% to 8% (e.g., about 2.5%, 3%, 3.5%, 4%, 4.5%, 5%, 5.5%, 6%, 6.5%, 7%, 7.5%, or 8%) (weight/volume). In embodiments, the resorcinol is present at a concentration of less than 2% but greater than about 0.1%, 0.5%, 1%, 1.5% (weight/volume). In embodiments, the resorcinol monoacetate is present at a concentration of less than 3% but greater than about 0.1%, 0.5%, 1%, 1.5%, 2%, or 2.5% (weight/volume).

In embodiments, a composition provide herein includes a moisturizer.

### Methods of Treating Acne

In an aspect, provided herein is a method of preventing or treating acne in a subject in need thereof, the method including administering an effective amount of a composition or combination provided herein. In embodiments, an effective amount of a composition comprising, consisting essentially of, or consisting of at least one P. acnes bacteriophage, at least one anti-acne compound, and a pharmaceutically acceptable carrier is administered to the subject. In embodiments, an effective amount of a composition that includes at least one P. acnes bacteriophage, at least one anti-acne compound and a pharmaceutically acceptable carrier, wherein the composition does not comprise a probiotic bacterium, is administered to the subject. In embodiments, an effective amount of a composition that includes a P. acnes bacteriophage and an enzyme is administered to the subject.

In embodiments, an effective amount of a composition that includes a bacteriophage as described herein, including embodiments thereof, is administered to the subject. In embodiments, the bacteriophage is a wild-type bacteriophage.

In embodiments, the bacteriophage is administered topically. In embodiments, the bacteriophage is in a composition (e.g., a pharmaceutical or cosmetic composition) that further includes a pharmaceutically or cosmetically acceptable carrier.

In embodiments, the method further includes administering a probiotic bacterium to the subject.

In an aspect, a method of treating acne in a subject in need thereof is provided. The method includes administering an effective amount of a probiotic P. acnes bacterium to the subject. In embodiments, the method further includes administering a bacteriophage to the subject.

In embodiments, the P. acnes bacterium has a 16S rDNA sequence that includes a T992C, T838C, C1322T, and/or a C986T mutation compared to the KPA171202 type strain 16S rDNA sequence set forth as SEQ ID NO: 2. In embodiments, the P. acnes bacterium includes a 16S rDNA sequence with a T838C and a C1322T mutation compared to the KPA171202 type strain 16S rDNA sequence set forth as SEQ ID NO: 2. In embodiments, the P. acnes bacterium is the ProI strain. In embodiments, the P. acnes bacterium includes a 16S rDNA sequence with a C986T and a T992C mutation compared to the KPA171202 type strain 16S rDNA sequence set forth as SEQ ID NO: 2. In embodiments, the P. acnes bacterium is the ProII strain.

In embodiments, the P. acnes bacterium: (a) includes a 16S rDNA sequence with a T992C mutation compared to the KPA171202 type strain 16S rDNA sequence set forth as SEQ ID NO: 2; (b) includes a 16S rDNA sequence with a T838C mutation compared to the KPA171202 type strain 16S rDNA sequence set forth as SEQ ID NO: 2; (c) includes a 16S rDNA sequence with a C1322T mutation compared to the KPA171202 type strain 16S rDNA sequence set forth as SEQ ID NO: 2; (d) includes a 16S rDNA sequence with a C986T mutation compared to the KPA171202 type strain 16S rDNA sequence set forth as SEQ ID NO: 2; (e) includes a 16S rDNA sequence that is identical to the sequence of SEQ ID NO: 3; (g) includes a 16S rDNA sequence that is identical to the sequence of SEQ ID NO: 4; (h) does not comprise a linear plasmid; (i) does not include a plasmid that includes a virulence factor; and/or (j) does not include a plasmid that encodes an extrachromosomal lipase and/or a tight adhesion virulence factor.

In embodiments, the method further includes administering at least one additional probiotic bacterium to the subject.

In embodiments, the at least one additional bacterium comprises, consists essentially of, or consists of a probiotic bacterium. In embodiments, the at least one bacterium includes 1, 2, 3, 4, 5, 6, 7, 8, 9, or 10 bacterial strains and/or species, less than about 10, 9, 8, 7, 6, 5, 4, 3, or 2 bacterial strains and/or species, or 1-10, 2-10, 3-10, 4-10, 5-10, 1-5, 2-5, 3-5, or 4-5 bacterial strains and/or species. In embodiments, the at least one bacterium includes a plurality of bacterial strains and/or species, e.g., at least about 2, 3, 4, 5, 6, 7, 8, 9, 10 bacterial strains and/or species. In embodiments, the least one bacterium includes a Propionibacterium sp., Staphylococcus sp., and/or Corynebacterium sp. bacterium. In embodiments, the least one bacterium includes bacterium within the class Betaproteobacteria. In embodiments, the least one bacterium includes an isolated Propionibacterium granulosum bacterium, an isolated Propionibacterium avidum bacterium, an isolated Staphylococcus epidermidis bacterium, an isolated Staphylococcus aureus bacterium, and/or an isolated Corynebacterium jeikeium bacterium. In embodiments, the least one bacterium includes 1, 2, 3, 4, or 5 of an isolated Propionibacterium granulosum bacterium, an isolated Propionibacterium avidum bacterium, an isolated Staphylococcus epidermidis bacterium, an isolated Staphylococcus aureus bacterium, and/or an isolated Corynebacterium jeikeium bacterium.

In embodiments, the subject has been administered a bacteriophage as described herein, including embodiments thereof.

In embodiments, the subject has been administered an antibiotic that kills P. acnes. In embodiments, the antibiotic is clindamycin, doxycycline, erythromycin, or tetracycline, or a derivative of clindamycin, doxycycline, erythromycin, or tetracycline.

In embodiments, the antibiotic is clindamycin, doxycycline, erythromycin, or tetracycline, or a derivative of clindamycin, doxycycline, erythromycin, or tetracycline.

In embodiments, the method further includes administering an enzyme to the subject such as a biofilm degradation enzyme or an anti-aging enzyme. Non-limiting examples of biofilm degradation enzymes include DNAses (e.g., DNAse I), restriction endonucleases, proteases (e.g., papain, bromelain, Trypsin, Proteinase K, Subtilisin, or serratiopeptidase), glycosidases (e.g., dispersin, alginate lyase, amylase, or cellulase). Non-limiting examples of anti-aging enzymes include superoxide dismutase, and peroxidase.

In embodiments, the method further includes administering a topical retinoid, an antibiotic, and/or an alpha-hydroxy acid. In embodiments, the method further includes administering a topical retinoid. In embodiments, the method further includes administering an antibiotic. In embodiments, the method further includes administering an alpha-hydroxy acid. In embodiments, the method further includes administering benzoyl peroxide, salicylic acid, sulfur, resorcinol, and/or resorcinol monoacetate to the subject. In embodiments, the method further includes administering benzoyl peroxide. In embodiments, the method further includes administering salicylic acid. In embodiments, the method further includes administering sulfur. In embodiments, the method further includes administering resorcinol and/or sulfur. In embodiments, the method further includes administering resorcinol and/or resorcinol monoacetate.

In embodiments, the method further includes administering an enhancing peptide or enzyme. In embodiments, the enhancing peptide or enzyme has one or more or any combination of the following properties: biofilm degradation, improving skin penetration, antibacterial, reducing inflammation (e.g., of the skin), reducing irritation (e.g., of the skin), reducing redness (e.g., of the skin), firming skin, removing lines, removing wrinkles, or otherwise improving appearance (e.g., of the skin).

In embodiments, at least about 2, 3, 4, 5, 6, 7, 8, 9, or 10P. acnes bacteriophages are administered to the subject. In embodiments, the P. acnes bacteriophages include more than one types of P. acnes bacteriophage.

### Exemplary Methods and Compositions for Treating Acne

In an aspect, provided herein is a composition that includes a bacteriophage. In embodiments, the bacteriophage is present in a composition, such as a therapeutic or cosmetic composition. In embodiments, the composition further includes a strain of probiotic bacteria. In embodiments, the composition further includes an enzyme that degrades a bacterial biofilms (e.g., a component thereof) in or on human skin pores. In embodiments, the enzyme enhances penetration of the bacteriophage and/or the probiotic bacteria. In embodiments, a bacteriophage (“phage”) destroys an acne-causing (i.e., pathogenic) strain of P. acnes with a high degree of specificity and efficacy, without killing beneficial skin bacteria. In embodiments, the biofilm-degrading enzyme dissolves the biofilm to increase the susceptibility of the pathogen (e.g., by reducing pathogen adherence to host cells and/or by increasing access of the bacteriophage to pathogenic cells). In embodiments, the probiotic bacteria are immune to the bacteriophage (e.g., the bacteria lack a cellular receptor to which the bacteriophage specifically binds). In embodiments, the probiotic bacteria occupy the niche left by a killed P. acnes pathogenic strain. In embodiments the probiotic bacteria reduce or prevent the recolonization or growth of a subject's skin (such as a pore) by surviving pathogenic bacteria.

In an aspect a composition for the therapeutic treatment of the skin disease acne is provided. In embodiments, the composition includes a lytic P. acnes bacteriophage, and optionally a probiotic bacterium sourced from healthy skin, and/or optionally a biofilm-degrading enzyme in the composition as an adjuvant to increase penetration of the active components.

In embodiments, a lytic P. acnes bacteriophage infects virulent P. acnes in a skin comedone. In embodiments, the bacteriophage replicates and lyses within the P. acnes. In embodiments, when the P. acnes lyses, it releases new virions. In embodiments, enzymes unclog the blocked comedones, dissolve the P. acnes biofilms and increase access of virions to P. acnes. In embodiments, the exponential proliferation of lytic P. acnes phages rapidly kills the P. acnes with high specificity, without disturbing the growth beneficial skin commensal bacteria. In embodiments, the niche vacated by the P. acnes is then be filled by the probiotic bacteria. In embodiments, the bacteria are sourced from healthy skin and expand to occupy the niche, thereby preventing any surviving P. acnes bacteria from growing back. In embodiments, this strategy helps to balance the skin microbiome in subjects and recalibrates their microbiome toward a healthy skin bacterial community. In embodiments, the biofilm-degrading enzyme is in a formulation as an adjuvant that helps unclog blocked comedones and increase access of the phage and probiotic bacteria to the pores.

In an aspect, a combination that includes a bacteriophage, a probiotic bacterium, and (optionally) an enzyme that enhances the penetration of the bacteriophage is provided. In embodiments, the pathogens are killed and the probiotic bacterium replaces the pathogen. In embodiments, a “kill and replace” approach to is used to treat acne. In embodiments, a biologic that selectively kills pathogenic bacteria that cause acne is administered to a subject. In embodiments, probiotic bacteria sourced from healthy skin are applied to occupy the niche of the killed pathogen. In embodiments, this approach avoids the problems of rampant drug resistance associated with antibiotics. In embodiments, the presence of actively dividing probiotic bacteria prevents relapses by not allowing any pathogens to grow back. In embodiments, dysbiosis on the skin of the subject is treated. In embodiments, a microbiome associated with acne is recalibrated into a healthy one.

In embodiments, the bacteriophage is a naturally occurring P. acnes bacteriophage.

Non-limiting examples of enzymes that may be co-administered with a bacteriophage include BL00275 from Bacillus licheniformis; DNase I; restriction endonucleases; deoxyribonucleases (e.g. from Staphylococcus aureus thermonuclease, B. licheniformis NucB, DNase 1L2); glycoside hydrolases (e.g. Dispersin B, alginate lyase, amylase, cellulase, glycanase); and proteases (e.g. subtlisin, proteinase K, trypsin, serratiopeptidase).

Non-limiting examples of probiotic bacteria that may be administered or present in a system or composition include one or more or any combination of the following bacterial species: Propionibacterium acnes, Propionibacterium granulosum, Propionibacterium avidum, Staphylococcus epidermidis, Staphylococcus aureus, and Corynebacterium jeikeium. In embodiments, a probiotic bacterial strain is be selected based on its ability to (a) colonize the skin without eliciting an adverse immune response, characterized by low lipase activity and reduced adhesion to human keratinocytes; and (b) occupy a niche similar to Propionibacterium acnes.

In embodiments, a biofilm degrading enzyme is present in the formulation and acts as an adjuvant, to increase the efficacy of the active ingredients (such as a bacteriophage). In embodiments, the enzyme has the capacity to degrade P. acnes biofilms in vitro.

### EMBODIMENTS

Embodiments and examples are provided below to facilitate a more complete understanding of the invention. The following embodiments and examples illustrate the exemplary modes of making and practicing the invention. However, the scope of the invention is not limited to specific embodiments disclosed in these embodiments and examples, which are for purposes of illustration only, since alternative methods can be utilized to obtain similar results.

Embodiments include Embodiments P1 to P56 following:
Embodiment P1. A composition consisting essentially of at least one Propionibacterium acnes bacteriophage, at least one anti-acne compound, and a pharmaceutically acceptable carrier.
Embodiment P2. A composition comprising at least one Propionibacterium acnes bacteriophage, at least one anti-acne compound, and a pharmaceutically acceptable carrier, wherein the composition does not comprise a probiotic bacterium.
Embodiment P3. The composition of Embodiment P2, wherein the composition further comprises a P. acnes biofilm degrading enzyme.
Embodiment P4. The composition of any one of Embodiments P1-P3, wherein the at least one anti-acne compound is benzoyl peroxide.
Embodiment P5. The composition of Embodiment P4, wherein the benzoyl peroxide is present at a concentration of 2.5% to 10% (weight/volume).
Embodiment P6. The composition of Embodiment P4, wherein the benzoyl peroxide is present at a concentration of less than 2.5% but greater than about 0.1%, 0.5%, 1%, 1.5%, or 2% (weight/volume).
Embodiment P7. The composition of any one of Embodiments P1-P3, wherein the at least one anti-acne compound is salicylic acid.
Embodiment P8. The composition of Embodiment P7, wherein the salicylic acid is present at a concentration of 0.5% to 2% (weight/volume).
Embodiment P9. The composition of Embodiment P7, wherein the salicylic acid is present at a concentration of less than 0.5% but greater than about 0.1% (weight/volume).
Embodiment P10. The composition of any one of Embodiments P1-P3, wherein the at least one anti-acne compound is sulfur.
Embodiment P11. The composition of Embodiment P10, wherein the sulfur is present at a concentration of 3% to 10% (weight/volume).
Embodiment P12. The composition of Embodiment P10, wherein the sulfur is present at a concentration of less than 3% but greater than about 0.1%, 0.5%, 1%, 1.5%, 2%, or 2.5% (weight/volume).
Embodiment P13. The composition of any one of Embodiments P1-P3, wherein the at least one anti-acne compound is resorcinol and sulfur.
Embodiment P14. The composition of Embodiment P13, wherein the resorcinol is present at a concentration of 2% and sulfur is present at a concentration of 3% to 8% (weight/volume).
Embodiment P15. The composition of any one of Embodiments P1-P3, wherein the at least one anti-acne compound comprises resorcinol monoacetate and sulfur.
Embodiment P16. The composition of Embodiment P15, wherein the resorcinol monoacetate is present at a concentration of 3% and sulfur is present at a concentration of 3% to 8% (weight/volume).
Embodiment P17. The composition of any one of Embodiments P1-P3, wherein the anti-acne compound is an antibiotic, a retinoid, or an alpha-hydroxy acid.
Embodiment P18. A composition comprising a Propionibacterium acnes bacteriophage and an enzyme.
Embodiment P19. The composition of any one of Embodiments P1-P18, wherein the P. acnes bacteriophage is a lytic P. acnes bacteriophage.
Embodiment P20. The composition of any one of Embodiments P1-P19, wherein the P. acnes bacteriophage comprises a linear double stranded DNA genome.
Embodiment P21. The composition of any one of Embodiments P1-P20, wherein the P. acnes bacteriophage is within the bacteriophage family Siphoviridae.
Embodiment P22. The composition of any one of Embodiments P1-P21, wherein the genome of the P. acnes bacteriophage comprises a nucleotide sequence that is at least about 80%, 85%, 90%, 95%, 96%, 97%, 98%, or 99% identical to the nucleotide sequence of SEQ ID NO: 1.
Embodiment P23. The composition of any one of Embodiments P18-P21, wherein the enzyme is a P. acnes biofilm degrading enzyme.
Embodiment P24. The composition of any one of Embodiments P3 or P18-P23, wherein the enzyme is a glycosidase, a protease, a DNAse, or a restriction endonuclease.
Embodiment P25. The composition of any one of Embodiments P3 or P18-P24, wherein the enzyme is a glycosidase.
Embodiment P26. The composition of Embodiment P25, wherein the glycosidase is a glycoside hydrolase.
Embodiment P27. The composition of Embodiment P26, wherein the enzyme catalyzes the hydrolysis of linear polymers of N-acetyl-D-glucosamines.
Embodiment P28. The composition of Embodiment P27, wherein the enzyme is a (3-hexosaminidase.
Embodiment P29. The composition of Embodiment P28, wherein the enzyme is hydrolyzes β-1,6-glycosidic linkages of acetylglucosamine polymers.
Embodiment P30. The composition of any one of Embodiments P3 or P18-P24, wherein the enzyme is a DNAse I, a restriction endonuclease, papain, bromelain, Trypsin, Proteinase K, Subtilisin, serratiopeptidase, dispersin, alginate lyase, amylase, or cellulase.
Embodiment P31. The composition of any one of Embodiments P3 or P18-P24, wherein the enzyme is Dispersin B.
Embodiment P32. The composition of any one of Embodiments P3 or P18-P24, wherein the enzyme is a protease, and the protease is proteinase K or subtilisin.
Embodiment P33. composition of any one of Embodiments P18-P22, wherein the enzyme is an anti-aging enzyme.
Embodiment P34. The composition of Embodiment P33, wherein the anti-aging enzyme is a superoxide dismutase or a peroxidase.
Embodiment P35. The composition of any one of Embodiments P18-P34, further comprising a probiotic bacterium.
Embodiment P36. The composition of Embodiment P35, wherein the probiotic bacterium is a probiotic a P. sp., Staphylococcus sp., and/or Corynebacterium sp. bacterium.
Embodiment P37. The composition of Embodiment P35, wherein the probiotic bacterium is a bacterium within the class Betaproteobacteria.
Embodiment P38. The composition of Embodiment P36, wherein the probiotic bacterium is a probiotic P. acnes bacterium.
Embodiment P39. The composition of Embodiment P38, wherein the P. acnes bacterium
(a) comprises a 16S DNA sequence with a T992C mutation compared to the KPA171202 type strain 16S DNA sequence set forth as SEQ ID NO: 2;
(b) comprises a 16S DNA sequence with a T838C mutation compared to the KPA171202 type strain 16S DNA sequence set forth as SEQ ID NO: 2;
(c) comprises a 16S DNA sequence with a C1322T mutation compared to the KPA171202 type strain 16S DNA sequence set forth as SEQ ID NO: 2;
(d) comprises a 16S DNA sequence with a C986T mutation compared to the KPA171202 type strain 16S DNA sequence set forth as SEQ ID NO: 2;
(e) comprises a 16S DNA sequence that is identical to the sequence of SEQ ID NO: 3;
(f) comprises a 16S DNA sequence that is identical to the sequence of SEQ ID NO: 4;
(g) does not comprise a linear plasmid;
(h) does not comprise a plasmid that comprises a virulence factor; and/or
(i) does not comprises a plasmid that encodes an extrachromosomal lipase and/or a tight adhesion virulence factor.
Embodiment P40. The composition of Embodiment P38, wherein the P. acnes bacterium:
(a) produces less than about 20% of the level of lipase that is produced by a pathogenic P. acnes strain when grown in a planktonic culture;
(b) produces less than about 10% of the level of lipase that is produced by a pathogenic P. acnes strain when grown in an adherent culture;
(c) adheres to epithelial cells at least 50% less than a pathogenic P. acnes strain; and/or
(d) is less inflammatory than a pathogenic P. acnes strain.
Embodiment P41. The composition of any one of Embodiments P35-P40, further comprising at least one additional probiotic bacterium.
Embodiment P42. The composition of Embodiment P41, wherein said at least one additional probiotic bacterium comprises Propionibacterium granulosum and/or Propionibacterium avidum.
Embodiment P43. The composition of Embodiment P40, wherein said pathogenic P. acnes strain (a) comprises a 16S DNA sequence with a G1058C mutation compared to the KPA171202 type strain 16S DNA sequence set forth as SEQ ID NO: 2; (b) comprises a 16S DNA sequence with a G1058C and an A1201C mutation compared to the KPA171202 type strain 16S DNA sequence set forth as SEQ ID NO: 2; (c) comprises a 16S DNA sequence with a G529A mutation compared to the KPA171202 type strain 16S DNA sequence set forth as SEQ ID NO: 2; (d) comprises a 16S DNA sequence with a G1004A and a T1007C mutation compared to the KPA171202 type strain 16S DNA sequence set forth as SEQ ID NO: 2; (e) comprises a 16S DNA sequence with a G1268A mutation compared to the KPA171202 type strain 16S DNA sequence set forth as SEQ ID NO: 2; (f comprises a 16S DNA sequence with a T554C and a G1058C mutation compared to the KPA171202 type strain 16S DNA sequence set forth as SEQ ID NO: 2; (g) comprises a 16S DNA sequence that is identical to the sequence of SEQ ID NO: 5; (h) comprises a 16S DNA sequence that is identical to the sequence of SEQ ID NO: 6; (i) comprises a 16S DNA sequence that is identical to the sequence of SEQ ID NO: 7; (j) comprises a 16S DNA sequence that is identical to the sequence of SEQ ID NO: 8; (k) comprises a 16S DNA sequence that is identical to the sequence of SEQ ID NO: 9; and/or (1) comprises a 16S DNA sequence that is identical to the sequence of SEQ ID NO: 10.
Embodiment P44. The composition of any one of Embodiments P18-P43, further comprising at least one additional P. acnes bacteriophage.
Embodiment P45. The composition of any one of Embodiments P1-P44, comprising a pharmaceutically acceptable carrier.
Embodiment P46. The composition of Embodiment P45, wherein the pharmaceutically acceptable carrier comprises an emulsion.
Embodiment P47. The composition of Embodiment P46, wherein the emulsion is an oil-in-water emulsion or a water-in-oil emulsion.
Embodiment P48. The composition of any one of Embodiments P1-P47, which is in the form of a cream, lotion, suspension, or aqueous solution.
Embodiment P49. A combination consisting essentially of at least one Propionibacterium acnes bacteriophage, and at least one anti-acne compound, wherein each of the at least one Propionibacterium acnes bacteriophage and the at least one anti-acne compound is in a composition that further comprises a pharmaceutically acceptable carrier.
Embodiment P50. The combination of Embodiment P49, wherein the at least one P. acnes bacteriophage and the at least one anti-acne compound are within separate compositions.
Embodiment P51. The combination of Embodiment P50, wherein the at least one P. acnes bacteriophage and the at least one anti-acne compound are within separate containers.
Embodiment P52. A combination comprising a Propionibacterium acnes bacteriophage and an enzyme.
Embodiment P53. The combination of Embodiment P52, wherein the P. acnes bacteriophage and the enzyme are within separate compositions.
Embodiment P54. The combination of Embodiment P53, wherein the P. acnes bacteriophage and the enzyme are within separate containers.
Embodiment P55. A method of treating acne in a subject in need thereof, the method comprising administering an effective amount of the composition of any one of Embodiments P1-P46 or the combination of any one of Embodiments P49-P54 to the subject.
Embodiment P56. The method of Embodiment P55, wherein the composition is administered topically.

Additional embodiments include Embodiments 1 to 55 following:
Embodiment 1. A composition comprising at least one Propionibacterium acnes bacteriophage, at least one anti-acne compound, and a pharmaceutically acceptable carrier.
Embodiment 2. The composition of Embodiment 1, which does not comprise a probiotic bacterium.
Embodiment 3. The composition of Embodiment 1 or 2, wherein the composition further comprises a P. acnes biofilm degrading enzyme.
Embodiment 4. The composition of any one of Embodiments 1-3, wherein the at least one anti-acne compound is salicylic acid.
Embodiment 5. The composition of Embodiment 4, wherein the salicylic acid is present at a concentration of 0.5% to 2% (weight/volume).
Embodiment 6. The composition of Embodiment 5, wherein the salicylic acid is present at a concentration of less than 0.5% but greater than about 0.1% (weight/volume).
Embodiment 7. The composition of any one of Embodiments 1-3, wherein the at least one anti-acne compound is sulfur.
Embodiment 8. The composition of Embodiment 7, wherein the sulfur is present at a concentration of 3% to 10% (weight/volume).
Embodiment 9. The composition of Embodiment 7, wherein the sulfur is present at a concentration of less than 3% but greater than about 0.1%, 0.5%, 1%, 1.5%, 2%, or 2.5% (weight/volume).
Embodiment 10. The composition of any one of Embodiments 1-3, wherein the at least one anti-acne compound is resorcinol and sulfur.
Embodiment 11. The composition of Embodiment 10, wherein the resorcinol is present at a concentration of 2% and sulfur is present at a concentration of 3% to 8% (weight/volume).
Embodiment 12. The composition of any one of Embodiments 1-3, wherein the at least one anti-acne compound comprises resorcinol monoacetate and sulfur.
Embodiment 13. The composition of Embodiment 12, wherein the resorcinol monoacetate is present at a concentration of 3% and sulfur is present at a concentration of 3% to 8% (weight/volume).
Embodiment 14. The composition of any one of Embodiments 1-3, wherein the anti-acne compound is an antibiotic, a retinoid, or an alpha-hydroxy acid.
Embodiment 15. The composition of any one of Embodiments 1-14, wherein the Propionibacterium acnes bacteriophage is a naturally occurring Propionibacterium acnes bacteriophage.
Embodiment 16. The composition of any one of Embodiments 1-15, wherein the P. acnes bacteriophage is a lytic P. acnes bacteriophage.
Embodiment 17. The composition of any one of Embodiments 1-16, wherein the P. acnes bacteriophage comprises a linear double stranded DNA genome.
Embodiment 18. The composition of any one of Embodiments 1-17, wherein the P. acnes bacteriophage is within the bacteriophage family Siphoviridae.
Embodiment 19. The composition of any one of Embodiments 1-19, wherein the genome of the P. acnes bacteriophage comprises a nucleotide sequence that is at least about 80%, 85%, 90%, 95%, 96%, 97%, 98%, or 99% identical to the nucleotide sequence of SEQ ID NO: 1.
Embodiment 20. The composition of any one of Embodiments 3-19, wherein the enzyme is a P. acnes biofilm degrading enzyme.
Embodiment 21. The composition of any one of Embodiments 3-20, wherein the enzyme is a glycosidase, a protease, a DNAse, or a restriction endonuclease.
Embodiment 22. The composition of any one of Embodiments 3-21, wherein the enzyme is a glycosidase.
Embodiment 23. The composition of Embodiment 22, wherein the glycosidase is a glycoside hydrolase.
Embodiment 24. The composition of Embodiment 23, wherein the enzyme catalyzes the hydrolysis of linear polymers of N-acetyl-D-glucosamines.
Embodiment 25. The composition of Embodiment 24, wherein the enzyme is a (3-hexosaminidase.
Embodiment 26. The composition of Embodiment 25, wherein the enzyme is hydrolyzes β-1,6-glycosidic linkages of acetylglucosamine polymers.
Embodiment 27. The composition of any one of Embodiments 3-20, wherein the enzyme is a DNAse I, a restriction endonuclease, papain, bromelain, Trypsin, Proteinase K, Subtilisin, serratiopeptidase, dispersin, alginate lyase, amylase, or cellulase.
Embodiment 28. The composition of any one of Embodiments 3-20, wherein the enzyme is Dispersin B.
Embodiment 29. The composition of any one of Embodiments 3-20, wherein the enzyme is a protease, and the protease is proteinase K or subtilisin.
Embodiment 30. The composition of any one of Embodiments 1-29, further comprising an anti-aging enzyme.
Embodiment 31. The composition of Embodiment 30, wherein the anti-aging enzyme is a superoxide dismutase or a peroxidase.
Embodiment 32. The composition of any one of Embodiments 1-31, further comprising a probiotic bacterium.
Embodiment 33. The composition of Embodiment 32, wherein the probiotic bacterium is a probiotic a P. sp., Staphylococcus sp., and/or Corynebacterium sp. bacterium.
Embodiment 34. The composition of Embodiment 32, wherein the probiotic bacterium is a bacterium within the class Betaproteobacteria.
Embodiment 35. The composition of Embodiment 33, wherein the probiotic bacterium is a probiotic P. acnes bacterium.
Embodiment 36. The composition of Embodiment 35, wherein the P. acnes bacterium
(a) comprises a 16S rDNA sequence with a T992C mutation compared to the KPA171202 type strain 16S rDNA sequence set forth as SEQ ID NO: 2;
(b) comprises a 16S rDNA sequence with a T838C mutation compared to the KPA171202 type strain 16S rDNA sequence set forth as SEQ ID NO: 2;
(c) comprises a 16S rDNA sequence with a C1322T mutation compared to the KPA171202 type strain 16S rDNA sequence set forth as SEQ ID NO: 2;
(d) comprises a 16S rDNA sequence with a C986T mutation compared to the KPA171202 type strain 16S rDNA sequence set forth as SEQ ID NO: 2;
(e) comprises a 16S rDNA sequence that is identical to the sequence of SEQ ID NO: 3;
(f) comprises a 16S rDNA sequence that is identical to the sequence of SEQ ID NO: 4;
(g) does not comprise a linear plasmid;
(h) does not comprise a plasmid that comprises a virulence factor; and/or
(i) does not comprises a plasmid that encodes an extrachromosomal lipase and/or a tight adhesion virulence factor.
Embodiment 37. The composition of Embodiment 35 or 36, wherein the P. acnes bacterium:
(a) produces less than about 20% of the level of lipase that is produced by a pathogenic P. acnes strain when grown in a planktonic culture;
(b) produces less than about 10% of the level of lipase that is produced by a pathogenic P. acnes strain when grown in an adherent culture;
(c) adheres to epithelial cells at least 50% less than a pathogenic P. acnes strain; and/or
(d) is less inflammatory than a pathogenic P. acnes strain.
38. The composition of any one of Embodiments 32-37, further comprising at least one additional probiotic bacterium.
Embodiment 39. The composition of Embodiment 38, wherein said at least one additional probiotic bacterium comprises Propionibacterium granulosum and/or Propionibacterium avidum.
Embodiment 40. The composition of Embodiment 37, wherein said pathogenic P. acnes strain
(a) comprises a 16S rDNA sequence with a G1058C mutation compared to the KPA171202 type strain 16S rDNA sequence set forth as SEQ ID NO: 2;
(b) comprises a 16S rDNA sequence with a G1058C and an A1201C mutation compared to the KPA171202 type strain 16S rDNA sequence set forth as SEQ ID NO: 2;
(c) comprises a 16S rDNA sequence with a G529A mutation compared to the KPA171202 type strain 16S rDNA sequence set forth as SEQ ID NO: 2;
(d) comprises a 16S rDNA sequence with a G1004A and a T1007C mutation compared to the KPA171202 type strain 16S rDNA sequence set forth as SEQ ID NO: 2;
(e) comprises a 16S rDNA sequence with a G1268A mutation compared to the KPA171202 type strain 16S rDNA sequence set forth as SEQ ID NO: 2;
(f comprises a 16S rDNA sequence with a T554C and a G1058C mutation compared to the KPA171202 type strain 16S rDNA sequence set forth as SEQ ID NO: 2;
(g) comprises a 16S rDNA sequence that is identical to the sequence of SEQ ID NO: 5;
(h) comprises a 16S rDNA sequence that is identical to the sequence of SEQ ID NO: 6;
(i) comprises a 16S rDNA sequence that is identical to the sequence of SEQ ID NO: 7;
(j) comprises a 16S rDNA sequence that is identical to the sequence of SEQ ID NO: 8;
(k) comprises a 16S rDNA sequence that is identical to the sequence of SEQ ID NO: 9; and/or
(l) comprises a 16S rDNA sequence that is identical to the sequence of SEQ ID NO: 10.
Embodiment 41. The composition of any one of Embodiments 1-40, further comprising at least one additional P. acnes bacteriophage.
Embodiment 42. The composition of any one of Embodiments 1-41, wherein the pharmaceutically acceptable carrier comprises an emulsion.
Embodiment 43. The composition of Embodiment 42, wherein the emulsion is an oil-in-water emulsion or a water-in-oil emulsion.
Embodiment 44. The composition of any one of Embodiments 1-44, which is in the form of a cream, lotion, suspension, or aqueous solution.
Embodiment 45. A combination comprising at least one Propionibacterium acnes bacteriophage and at least one anti-acne compound, wherein each of the at least one Propionibacterium acnes bacteriophage and the at least one anti-acne compound is in a composition that further comprises a pharmaceutically acceptable carrier.
Embodiment 46. The combination of Embodiment 45, wherein the at least one P. acnes bacteriophage and the at least one anti-acne compound are within separate compositions.
Embodiment 47. The combination of Embodiment 46, wherein the at least one anti-acne compound is benzoyl peroxide.
Embodiment 48. The combination of Embodiment 47, wherein the benzoyl peroxide is present at a concentration of 2.5% to 10% (weight/volume).
Embodiment 49. The combination of Embodiment 47, wherein the benzoyl peroxide is present at a concentration of less than 2.5% but greater than about 0.1%, 0.5%, 1%, 1.5%, or 2% (weight/volume).
Embodiment 50. A method of treating acne in a subject in need thereof, the method comprising administering an effective amount of the composition of any one of Embodiments 1-44 to the subject.
Embodiment 51. The method of Embodiment 50, wherein the composition is administered topically.
Embodiment 52. A method of treating acne in a subject in need thereof, the method comprising administering an effective amount of the combination of any one of Embodiments 45-49 to the subject.
Embodiment 53. A composition comprising a Propionibacterium acnes bacteriophage and an enzyme.
Embodiment 54. A combination comprising a Propionibacterium acnes bacteriophage and an enzyme.
Embodiment 55. A composition consisting essentially of at least one Propionibacterium acnes bacteriophage, at least one anti-acne compound, and a pharmaceutically acceptable carrier.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1. P. Acnes Bacteriophage PHIT-101 Kills P. Acnes Selectively and Efficiently

P. acnes bacteriophages have been shown to be genetically highly similar and exhibit a broad range against multiple strains of P. acnes. A lead bacteriophage (PHIT-101) was used for experimentation. PHIT-101 is a single lytic phage that killed all the strain types of P. acnes tested (data not shown). PHIT-101 has the sequence of SEQ ID NO: 1. In order to showcase the efficacy and specificity of this phage, a plate assay was performed as follows. P. acnes KPA171202 and P. granulosum (a closely related but benign skin bacterium) were plated on separate BHI-agar plates. Sterile cotton pads were placed on each plate. The sterile cotton pads were soaked in either minocycline, an antibiotic commonly used to treat acne, or a phage solution with a titer of 2×10⁷ pfu/mL. After incubating the plates anaerobically for 72 hours at 37° C., the minocycline pads killed bacteria indiscriminately, showing a zone of killing on both the acne-causing P. acnes and the commensal P. granulosum (FIG. 1). In contrast, the PHIT-101 pads killed only the P. acnes, without disturbing the growth of beneficial P. granulosum.

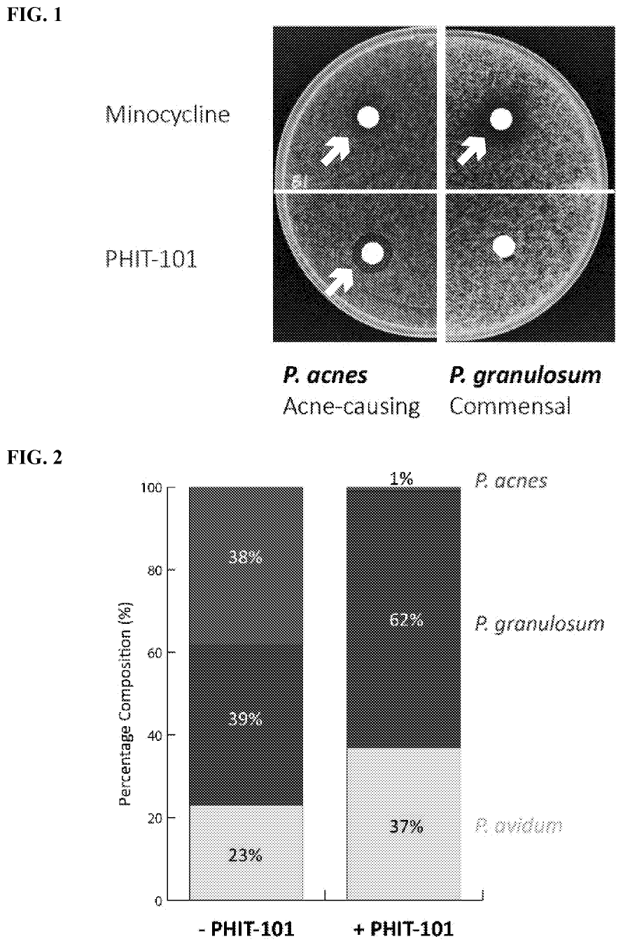

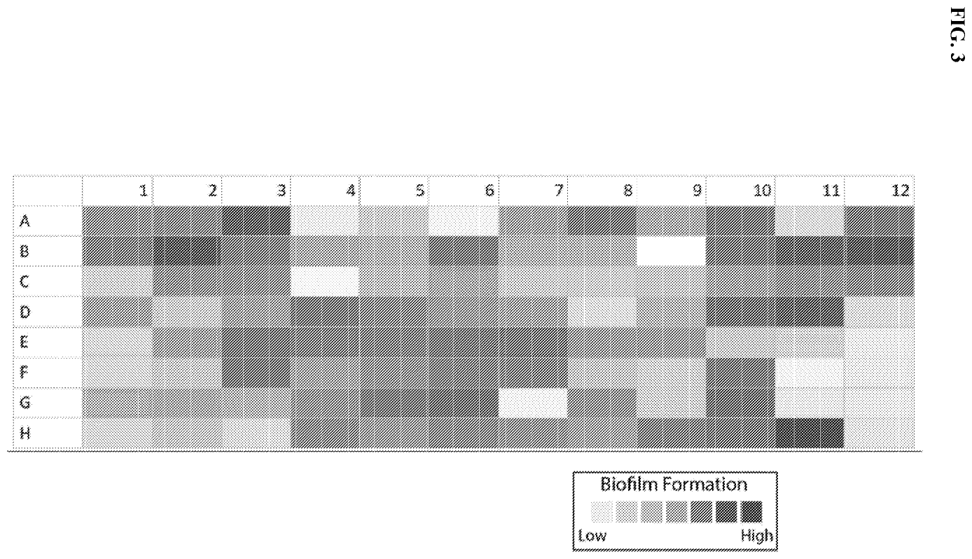

Further evidence of the ability of PHIT-101 to kill selectively was obtained in a synthetic skin microbiome assay. A synthetic skin microbiome was formulated comprising P. acnes, P. granulosum, and P. avidum, three skin bacteria that comprise 60-80% of microbiota in the skin pore [Science (2009) 324:1190-1192]. This synthetic skin microbiome was grown anaerobically in the presence or absence of PHIT-101 (final concentration 5×10⁵ pfu/mL). After 48 hours of incubation at 37° C., the cells were pelleted and washed, and the relative proportions of the three species was determined using 16S amplicon next-generation sequencing (NGS) on Illumina MiSeq. The results in FIG. 2 show that PHIT-101 is able to kill P. acnes almost completely, without negatively affecting the growth of the commensal P. granulosum and P. avidum.

Screening Biofilm Degrading Enzymes (BDEs) to Disrupt P. acnes Biofilms.

Several recent reports (Exp Dermatol (2014) 23:687, Br J Dermatol (2015) 172:13) have established that P. acnes produces significant amounts of biofilm in skin pores, which prevents antibiotic penetration and results in poor treatment outcomes. In order to validate this, biofilm production of several strains of P. acnes was quantified. FIG. 3 shows that adherent cultures of multiple strains isolated from the microbiota of a single subject produce markedly different levels of biofilm under similar conditions. Thus the previous proof-of-concept using planktonic cells did not reflect the true conditions under which P. acnes grows on the skin.

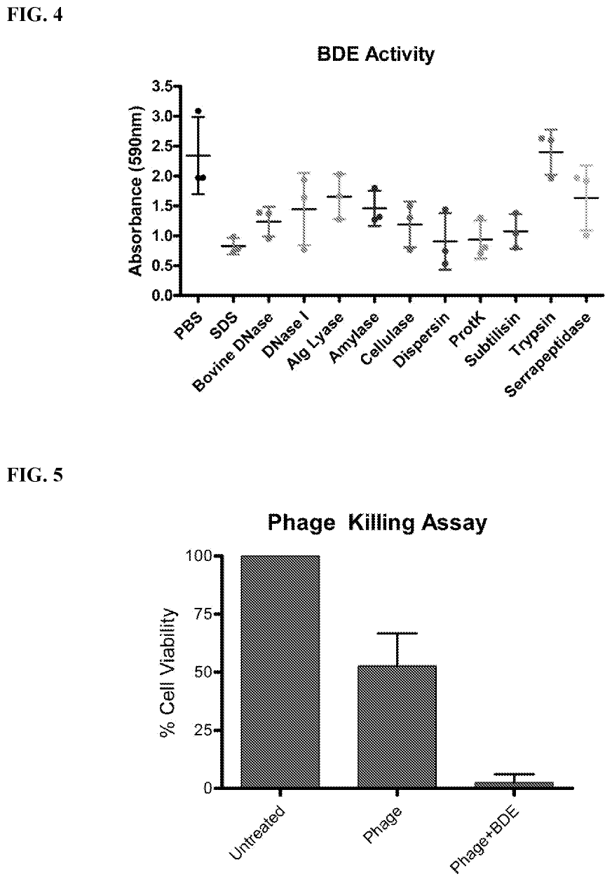

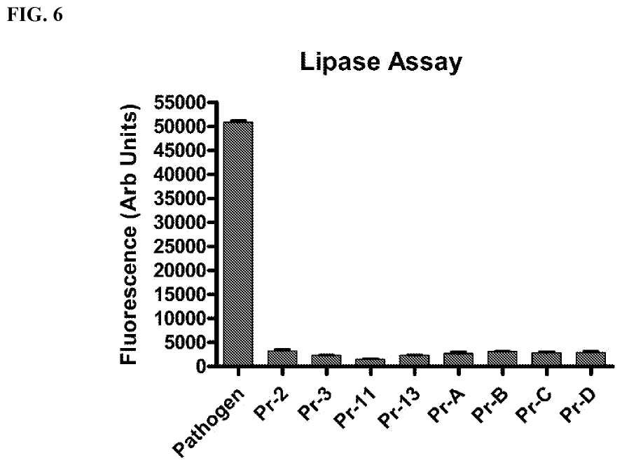

Without being bound by any scientific theory, we hypothesized that biofilms might present a significant barrier to phage killing of sessile P. acnes cells. This hypothesis was validated in a cell survival assay (FIG. 5) which showed that unlike planktonic P. acnes (99% killing, FIG. 2), PHIT-101 was only able to kill about 50% of the P. acnes cells encased in biofilms. In order to determine whether biofilm degradation would improve phage killing, a number of enzymes was screened to find a BDE specific for P. acnes. The screen comprised three classes of enzymes that might degrade types of materials that may be found in biofilms: DNA, polysaccharides, and proteins. FIG. 4 shows that in the screen, DNAses had moderate activity while the best rates of biofilm degradation were found in proteases and dispersin, a glycoside hydrolase from Aggregatibacter actinomycetemcomitans.

In selecting the BDE to pair with the phage, dispersin was selected for two reasons: firstly, as a glycoside hydrolase it was unlikely to attack the protein coat of the phage itself, thereby avoiding possible degradation of the phage. Secondly, P. acnes co-forms robust biofilms with Staphylococcus aureus [Anaerobe (2016) 40:63-67] and dispersin is active against biofilms from both organisms. Whether the addition of dispersin would increase the efficiency of phage killing in sessile P. acnes was determined. FIG. 5 shows that bacterial killing of PHIT-101 was enhanced in the presence of dispersin, restoring a ˜99% killing efficiency to the phage.

### Example 2. Probiotic Bacteria

### Genotypic Characterization of Probiotic Strains.

Strains of P. acnes were characterized based on point mutations in the 16S rDNA sequence which leads to phylogenetic sorting into pathogenic and probiotic strain types, and the absence of a linear plasmid found in pathogenic strains, which carries virulence factors. Using 16S-specific primers the full 16S rDNA sequence of each P. acnes strain was amplified and Sanger-sequenced. A probiotic strain was identified as having ribosequence (RS) of ProI or ProII. ProI strains have T838C and C1322T mutations relative to the KPA171202 type strain's 16S rDNA sequence (NIH Accession No. NC_006085.1). ProII strains have C986T and T992C mutations relative to the KPA171202 sequence. Further, using specific primer pairs, the presence or absence of a linear plasmid within each strain was determined. Probiotic strains were identified as lacking this plasmid, which carries an extrachromosomal lipase as well as the Tad (tight adhesion) virulence factor.

In embodiments, the probiotic strains are characterized primarily by their 16S sequences, e.g., SEQ ID NO: 3 and SEQ ID NO: 4. In embodiments, they can be genotypically identified by the lack of the plasmid bearing virulence factors, such as an extrachromosomal lipase and a Tad locus.

The cohort of probiotic strains was further characterized for their immunogenic potential. A lead probiotic candidate based on two factors: low lipase production, and less tight adherence to epithelial cells. The phenotypic validation of these features was important in selecting the probiotic lead candidate.

Testing the Immunogenic Potential of Probiotic P. acnes Strains: Lipase Activity.

Lipases play an important role in pathogenesis of acne by hydrolyzing sebum triglycerides and releasing irritating free fatty acids in the pilosebaceous follicles. Lipase is a strong chemotactic and proinflammatory antigen. Therefore, lipase is of high interest as a pharmacological target for anti-acne drugs. In embodiments, the overall strategy is to replace the pathogenic P. acnes that secretes high levels of lipase with a low-secreting probiotic P. acnes. In order to quantify the lipase expression phenotype for each strain in our panel, lipase production of the probiotic P. acnes strains was compared against pathogenic P. acnes strains with a fluorescent lipase activity assay.

One of the most interesting findings was that each strain secreted different amounts of lipases when grown in planktonic vs adherent culture. This has been previously reported in P. acnes strains [Res Microbiol, (2007) 158:386-392]. Further, the data showed that when these strains were grown in liquid culture, there was no significant difference between the lipase output of the pathogenic and probiotic strains. However, when these strains were grown under biofilm conditions, an interesting change was seen. While variability in production between strains could still be observed, several probiotic strains had significantly less lipase activity than pathogenic strains (FIG. 11). Interestingly, not all strains within the probiotic cohort had low lipase activity. For example, the lipase production of strains Pr-1 and Pr-5 was over the threshold for a probiotic strain, and was not developed further. Thus by quantifying lipase production in sessile P. acnes cells, it was possible to screen amongst probiotic strains and select those lead candidates with the most consistent low levels of lipase activity.

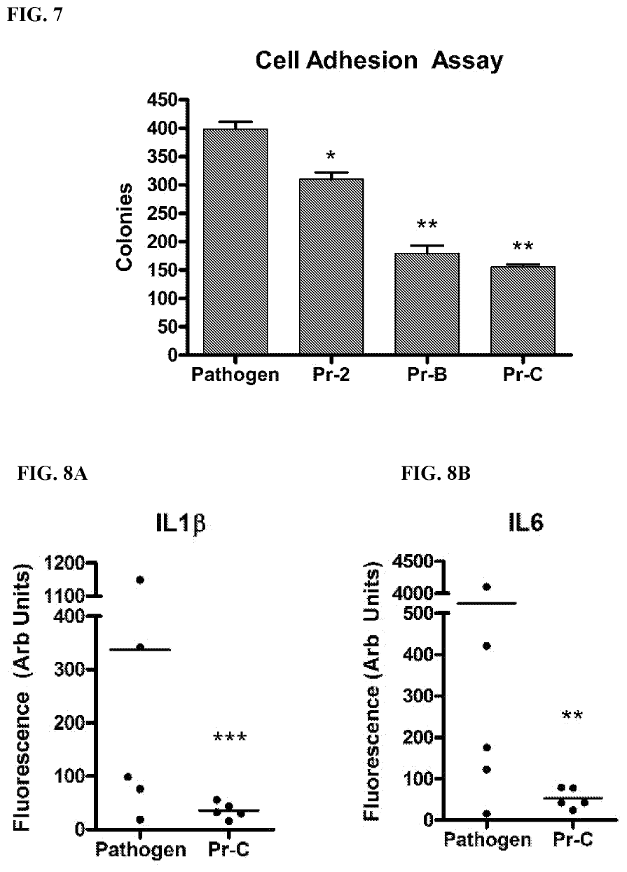

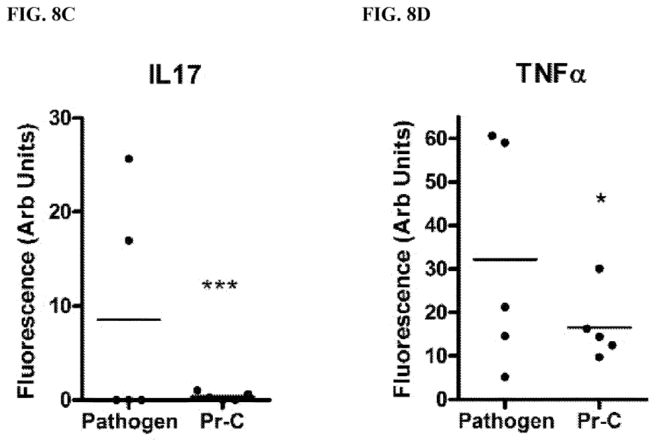

Thus, while pathogenic and probiotic strains secreted similar amounts of lipase in planktonic culture, the probiotic strains secreted far less lipase in adherent culture than pathogenic strains. FIG. 8 shows that the top probiotic candidates had a low lipase profile compared to the pathogenic strain.

Testing the Immunogenic Potential of Probiotic P. acnes Strains: Cell Adherence.

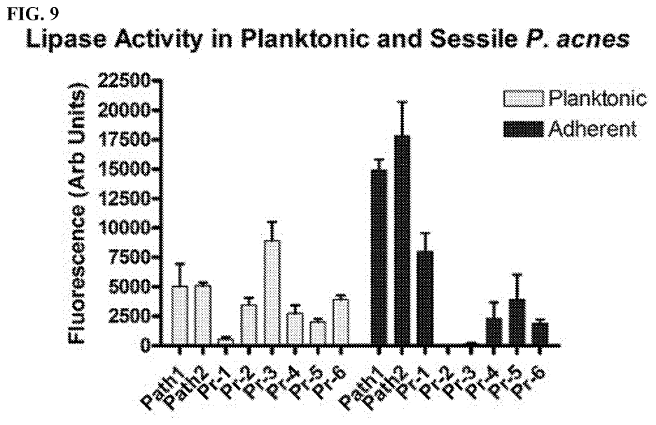

Available pathogenic strains were confirmed to possess a tight adhesion (tad) locus that plays a role in the virulence of other mammalian pathogens [J Bacteriol (2000) 182:6169-6176; Nat Rev Microbiol (2007) 5:363-375; PNAS (2003) 100:7295-7300]. Greater adherence to host cells may increase virulence or induce an inflammatory host response. The probiotic strains were previously genotypically verified to not contain the tad locus, and thus predicted to adhere less tightly to epithelial cells. The adhesion of pathogenic and probiotic strains to A-431 dermal epithelial cells was compared, in order to assess whether there was an appreciable difference in adherence. FIG. 9 shows that the top three probiotic candidates adhered less tightly to epithelial cells than the pathogenic strain. Interestingly, once again a subtle but persistent difference in cell adhesion was found between different strain families of P. acnes. Thus the strains of P. acnes with ProI ribosequence exhibited a slightly higher cell adherence (Pr-2 in FIG. 9) while the ProII strains adhered to cells less tightly (Pr-B, Pr-C in FIG. 9).

Comparison of Pathogenic and Probiotic P. acnes in Mouse Ear Inflammation Model.

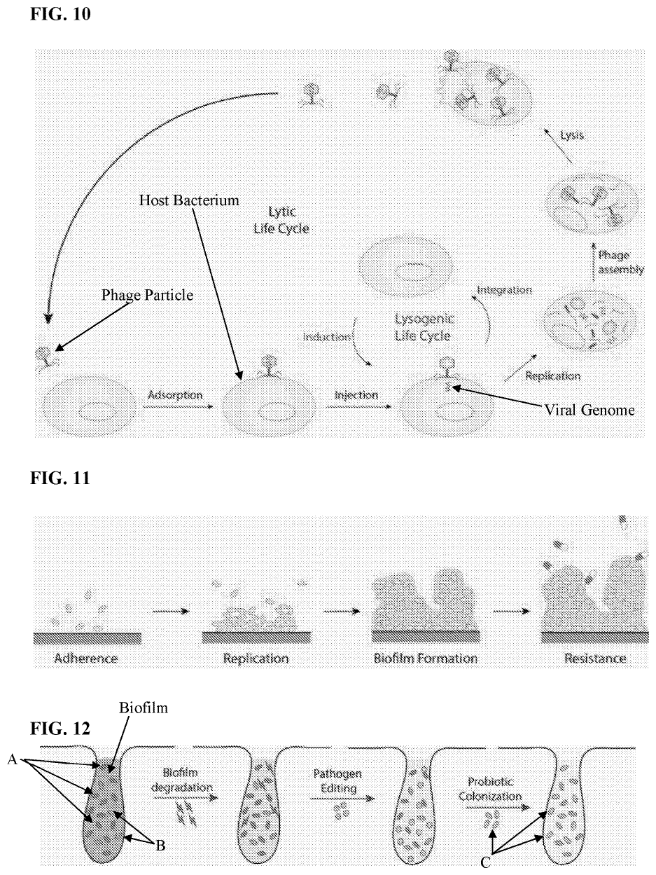

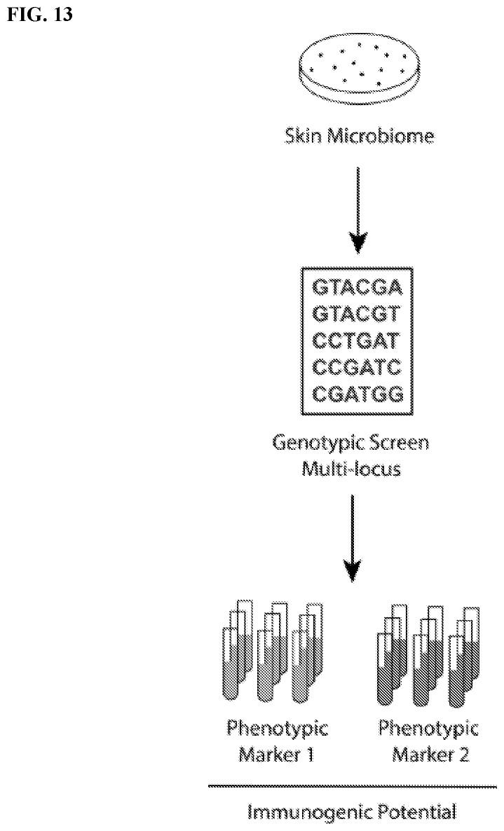

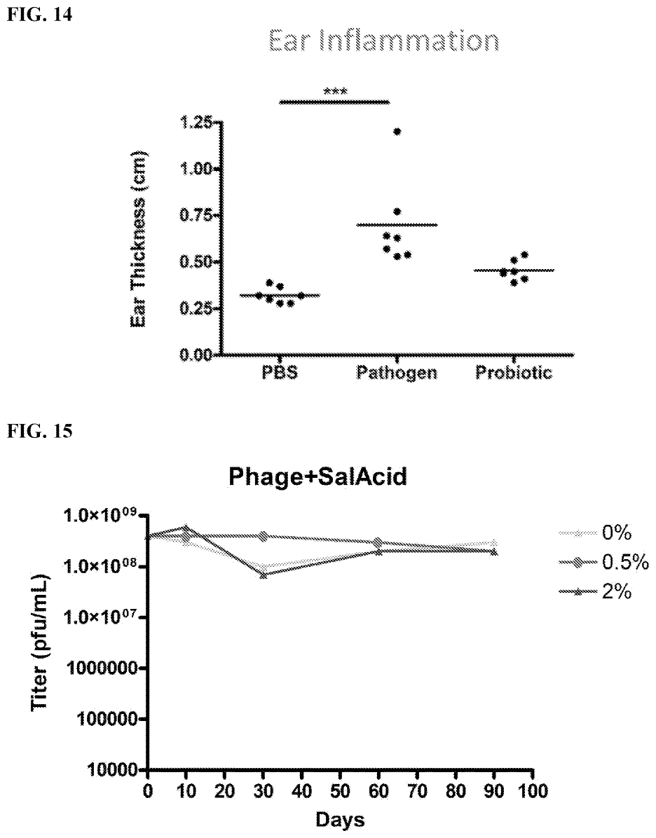

Upon validating the low immunogenic potential of the probiotic strains showing that they produced less lipase and adhered less tightly to epithelial cells, the inflammatory response of these strains was tested in a mouse ear inflammation model, which is well established and has been used previously to evaluate the inflammatory potential of P. acnes in the context of acne. The inflammatory potential of pathogenic and probiotic strains was compared in the following study: 10¹⁰ cfu of a strain was injected into the ears of CBA/J mice. A cohort of 5 mice was assigned to each strain. After 5 days the ears were excised and examined for inflammation. The levels of several inflammatory cytokines (IL-1 (3, IL-6, IL-17, TNFα) were measured and the sections of the tissue were examined by histology. FIG. 10 shows that the pathogenic strain had significantly higher levels of IL-1β, IL-6, IL-17, and TNFα compared to the probiotic strain.

### Acute Dermal Safety and Toxicity of Probiotic Strains in Miniswine Skin Model.

A miniswine model was used to test the probiotic strain for skin irritation. Swine are one of the major animals used in translational research, and pig skin is physiologically, anatomically, biochemically and immunologically similar to human skin. Miniswine are particularly commonly used to model human dermal diseases and conditions like acne [Vet Pathol (2012) 49:344-356]. The probiotic strain was applied to the skin of three separate miniswine in two doses—10⁸ cfu and 10⁹ cfu—in delimited skin areas. The animals were observed daily for clinical signs and the dosing site skin was scored using the Draize Scoring System at pre-dose, 0.5, 1, 4, 8, and 24 hours post dose administration. There was no erythema or edema associated with the lead probiotic strain during the entire period (Table 1), and a Draize score of 0 was observed throughout. This demonstrates the safety to acute exposure of our probiotic strain in an animal skin model.

TABLE 1
Acute dermal safety/tox in miniswine skin model shows good safety
profile of probiotic strain. Probiotic bacteria was applied at normal
(10⁸ cfu) and acute (10⁹ cfu) doses on delimited skin areas
in 3 male miniswine and monitored for 24 hours post-application.
Erythema and edema were quantified using the Draize Scoring
System. The Draize score provides the relative severity of
erythema and edema. A Draize score of 0, indicating complete
absence of erythema and edema, was observed on all the skin
areas throughout the monitoring period.

| Group | | | | Total Sites with non- |
|---|---|---|---|---|
| (Animal) | Dose Site | Treatment | Dose Level | zero Draize score* |
| 3 Male | Left #1 | P. acnes | ~10⁸ CFU | 0 |
| | | Normal | | |
| | Right #1 | P. acnes | ~10⁹ CFU | 0 |
| | | Acute | | |
| | Left #2 | PHIT-101 | ~10⁸ CFU | 0 |
| | | Normal | | |
| | Right #2 | PHIT-101 | ~10⁹ CFU | 0 |
| | | Acute | | |

### Example 3. Bacteriophage Stability in Compositions with Anti-Acne Compounds

In order to determine whether the phage was stable in co-formulation with either salicylic acid or benzoyl peroxide (BPO), the phage was co-incubated with these agents at a low and high concentration. The range of concentrations was determined by the permitted concentrations of these agents specified in the United States Food and Drug Administration (FDA) acne monograph for over-the-counter use. For salicylic acid, this is 0.5% to 2% (w/v), while for BPO the range is 2.5% to 10% (w/v). Buffered solutions of phage were added to these agents, and its stability at 4° C. was tested over 60-90 days. FIG. 15 shows that the phages are stable in the presence of both low and high doses of salicylic acid. In contrast, FIG. 16 shows that benzoyl peroxide destabilizes the phages, and the observed rate of decrease in phage viability is steeper at a higher concentration of BPO.

### Example 4 (Prophetic). Treatment with a Combination of Bacteriophage with Salicylic Acid

A double-blind, placebo-controlled study of a composition comprising Propionibacterium acnes bacteriophage and salicylic acid is conducted determine the comparative efficacy of this treatment with placebo, Propionibacterium acnes bacteriophage alone, and salicylic acid alone. Concentrations of 0.5% and 2% (w/v) salicylic acid are administered with and without Propionibacterium acnes bacteriophage. In all conditions that include the Propionibacterium acnes bacteriophage, the phage is present in a dose of 10⁹ pfu (plaque forming units) per dose. Ten subjects who have comparably severe acne are treated for each of the following groups:
(i) Placebo (no active agent)
(ii) 0.5% salicylic acid as the sole active agent
(iii) 2% salicylic acid as the sole active agent
(iv) Propionibacterium acnes bacteriophage as the sole active agent
(v) the combination of 0.5% salicylic acid and Propionibacterium acnes bacteriophage (in a single composition)
(vi) the combination of 2% salicylic acid and Propionibacterium acnes bacteriophage (in a single composition)

The combination of the Propionibacterium acne bacteriophage with salicylic acid achieves more than an additive effect, i.e., a synergistic effect (the combined effect of the bacteriophage and the salicylic acid is greater than the sum of the effects of the bacteriophage and the salicylic acid when each agent is used separately) in treating acne. The effectiveness of treatment is measured using lesion counts and an IGA (investigator global assessment) score.

### Example 5 (Prophetic). Treatment with a Combination of Bacteriophage with Sulfur

A double-blind, placebo-controlled study of a composition comprising Propionibacterium acnes bacteriophage and sulfur is conducted determine the comparative efficacy of this treatment with placebo, Propionibacterium acnes bacteriophage alone, and sulfur alone. Concentrations of 3% and 10% (w/v) sulfur are administered with and without Propionibacterium acnes bacteriophage. In all conditions that include the Propionibacterium acnes bacteriophage, the phage is present in a dose of 10⁹ pfu per dose. Ten subjects who have comparably severe acne are treated for each of the following groups:
(i) Placebo (no active agent)
(ii) 3% sulfur as the sole active agent
(iii) 10% sulfur as the sole active agent
(iv) Propionibacterium acnes bacteriophage as the sole active agent
(v) the combination of 3% sulfur and Propionibacterium acnes bacteriophage (in a single composition)
(vi) the combination of 10% sulfur and Propionibacterium acnes bacteriophage (in a single composition)

The combination of the Propionibacterium acne bacteriophage with sulfur achieves more than an additive effect, i.e., a synergistic effect (the combined effect of the bacteriophage and the sulfur is greater than the sum of the effects of the bacteriophage and the sulfur when each agent is used separately) in treating acne. The effectiveness of treatment is measured using lesion counts and an IGA (investigator global assessment) score.

### Example 6 (Prophetic). Treatment with a Combination of Bacteriophage with Benzoyl Peroxide

A double-blind, placebo-controlled study of a composition comprising Propionibacterium acnes bacteriophage and BPO is conducted determine the comparative efficacy of this treatment with placebo, Propionibacterium acnes bacteriophage alone, and BPO alone. Concentrations of 2.5% and 10% (w/v) BPO are administered with and without Propionibacterium acnes bacteriophage. In all conditions that include the Propionibacterium acnes bacteriophage, the phage is present in a dose of 10⁹ pfu per dose. Ten subjects who have comparably severe acne are treated for each of the following groups:
(i) Placebo (no active agent)
(ii) 2.5% BPO as the sole active agent
(iii) 10% BPO as the sole active agent
(iv) Propionibacterium acnes bacteriophage as the sole active agent
(v) the combination of 2.5% BPO and Propionibacterium acnes bacteriophage (in separate compositions)
(vi) the combination of 10% BPO and Propionibacterium acnes bacteriophage (in a single compositions)

The combination of the Propionibacterium acne bacteriophage with BPO achieves more than an additive effect, i.e., a synergistic effect (the combined effect of the bacteriophage and the BPO is greater than the sum of the effects of the bacteriophage and the BPO when each agent is used separately) in treating acne. The effectiveness of treatment is measured using lesion counts and an IGA (investigator global assessment) score.

### Example 7 (Prophetic). Assay with a Combination of Bacteriophage with Benzoyl Peroxide

An in vitro study is performed to determine the efficacy of (i) BPO; (ii) Propionibacterium acne bacteriophage; or (iii) Propionibacterium acne bacteriophage+BPO in killing planktonic and sessile pathogenic P. acnes bacteria.

The combination of the Propionibacterium acne bacteriophage with BPO achieves more than an additive effect, i.e., a synergistic effect (the combined effect of the bacteriophage and the BPO is greater than the sum of the effects of the bacteriophage and the BPO when each agent is used separately) in killing sessile pathogenic P. acnes bacteria. The keratolytic action of BPO (similar to salicylic acid and retinoids) assists the phage in penetrating skin pores to access the P. acnes deep within the pores.

1.-55. (canceled)

56. A composition formulated for topical administration comprising:
(a) a PHIT-101 bacteriophage;
(b) a dispersin enzyme; and
(c) a probiotic bacteria comprising a strain of Propionibacterium acnes (P. acnes), wherein the P. acnes strain comprises 16S rDNA sequences comprising SEQ ID NO: 3 and SEQ ID NO: 4.

57.-61. (canceled)

62. A method for treating acne in a subject in need thereof, the method comprising:
topically administering the composition of claim 77 to a region of the subject's skin comprising the acne.

63. The method of claim 62, comprising:
prior to topical administration, pretreating the region of the subject's skin with a composition comprising benzoyl peroxide.

64. The method of claim 62, wherein the benzoyl peroxide composition is washed off of the region of the subject's skin prior to the topical administration of the composition.

65. A method for treating acne in a subject in need thereof, the method comprising:
(a) topically administering a composition comprising a dispersin enzyme to a region of the subject's skin comprising the acne;
(b) topically administering the composition of claim 77 to the region of the subject's skin comprising the acne; and
(c) topically administering a composition comprising a strain of Propionibacterium acnes (P. acnes), wherein the P. acnes strain comprises 16S rDNA sequences comprising SEQ ID NO: 3 and SEQ ID NO: 4 to the region of the subject's skin comprising the acne.

66. The method of claim 65, wherein the dispersin enzyme comprises dispersin B.

67. (canceled)

68. The method of claim 65, wherein the dispersin B enzyme comprises SEQ ID NO: 12.

69. The method of claim 65, wherein before step (a), the region of the subject's skin comprising the acne is pretreated with benzoyl peroxide.

70. The method of claim 69, wherein the benzoyl peroxide is removed prior to step (a).

71. The method of claim 65, further comprising (d) topically administering a composition comprising one or more of salicylic acid, resorcinol, and sulfur to the region of the subject's skin comprising the acne.

72. The method of claim 65, wherein the composition comprising the bacteriophage further comprises salicylic acid.

73. The method of claim 65, wherein the composition comprising the dispersin enzyme and the composition comprising the bacteriophage are the same.

74. The method of claim 65, wherein the composition comprising the bacteriophage and the composition comprising the probiotic bacteria are the same.

75. The method of claim 74, wherein the composition comprising the bacteriophage and the probiotic bacteria also comprises salicylic acid.

76. (canceled)

77. A composition comprising:
(a) at least one Propionibacterium bacteriophage, wherein the genome of the Propionibacterium bacteriophage comprises a nucleotide sequence that is at least about 90%, 95%, 96%, 97%, 98%, or 99% identical to the nucleotide sequence of SEQ ID NO: 1;
(b) at least one anti-acne compound; and
(c) a pharmaceutically acceptable carrier.

78. The composition of claim 77, wherein the anti-acne compound is one or more of salicylic acid, benzoyl peroxide, and sulfur.

79. The composition of claim 77, wherein the Propionibacterium bacteriophage has one or more of the following properties:
a) it is a lytic Propionibacterium acnes (P. acnes) bacteriophage;
b) it is a linear double stranded DNA genome;
c) it is within the bacteriophage family Siphoviridae.

80. The composition of claim 77, further comprising a biofilm degrading enzyme.

81. The composition of claim 80, wherein the enzyme is a P. acnes biofilm degrading enzyme.

82. The composition of claim 80, wherein the enzyme is one of:
a glycosidase;
a protease;
a DNAse;
a restriction endonuclease;
catalyst of hydrolysis of linear polymers of N-acetyl-D-glucosamines;
a β-hexosaminidase;
hydrolyzes β-1,6-glycosidic linkages of acetylglucosamine polymers;
a restriction endonuclease;
papain;
bromelain;
Trypsin;
Proteinase K;
Subtilisin;
Serratiopeptidase;
Dispersin;
alginate lyase;
amylase;
cellulase;
Dispersin B;
a protease;
an anti-aging enzyme;
superoxide dismutase; or
a peroxidase.

83. The composition of claim 77, further comprising a probiotic bacterium, P. acnes bacterium comprising at least one of the following characteristics:
(a) comprises a 16S rDNA sequence with a T992C mutation compared to the KPA171202 type strain 16S rDNA sequence set forth as SEQ ID NO: 2;
(b) comprises a 16S rDNA sequence with a T838C mutation compared to the KPA171202 type strain 16S rDNA sequence set forth as SEQ ID NO: 2;
(c) comprises a 16S rDNA sequence with a C1322T mutation compared to the KPA171202 type strain 16S rDNA sequence set forth as SEQ ID NO: 2;
(d) comprises a 16S rDNA sequence with a C986T mutation compared to the KPA171202 type strain 16S rDNA sequence set forth as SEQ ID NO: 2;
(e) comprises a 16S rDNA sequence that is identical to the sequence of SEQ ID NO: 3;
(f) comprises a 16S rDNA sequence that is identical to the sequence of SEQ ID NO: 4;
(g) does not comprise a linear plasmid;
(h) does not comprise a plasmid that comprises a virulence factor;
(i) does not comprises a plasmid that encodes an extrachromosomal lipase and/or a tight adhesion virulence factor;
(j) produces less than about 20% of the level of lipase that is produced by a pathogenic P. acnes strain when grown in a planktonic culture;
(k) produces less than about 10% of the level of lipase that is produced by a pathogenic P. acnes strain when grown in an adherent culture;
(l) adheres to epithelial cells at least 50% less than a pathogenic P. acnes strain; or
(m) is less inflammatory than a pathogenic P. acnes strain.

84. The composition of claim 83, wherein the probiotic bacterium includes at least one of:
Propionibacterium. sp.;
Staphylococcus sp.;
Corynebacterium sp.;
a bacterium within class Betaproteobacteria;
Propionibacterium acnes;
Propionibacterium granulosum; or
Propionibacterium avidum.

85. A composition comprising:
at least one Propionibacterium bacteriophage, wherein the genome of the P. acnes bacteriophage comprises a nucleotide sequence that is at least about 90%, 95%, 96%, 97%, 98%, or 99% identical to the nucleotide sequence of SEQ ID NO: 1; and
at least one compound selected from salicylic acid, benzoyl peroxide, and sulfur.

86. The composition of claim 77, wherein at least one of the Propionibacterium bacteriophage is a Propionibacterium acnes (P. acnes) bacteriophage, wherein the genome of the P. acnes bacteriophage comprises a nucleotide sequence of SEQ ID NO: 1, and wherein the anti-acne compound is salicylic acid.

87. The composition of claim 85, wherein the genome of the P. acnes bacteriophage comprises a nucleotide sequence of SEQ ID NO: 1, and wherein the anti-acne compound is sulfur.

88. The composition of claim 85, wherein the genome of the P. acnes bacteriophage comprises a nucleotide sequence of SEQ ID NO: 1, and wherein the anti-acne compound is benzoyl peroxide.

89. A method for treating acne in a subject in need thereof, the method comprising:
topically administering the composition of claim 85 to a region of the subject's skin comprising the acne.

90. The composition of claim 77, wherein the genome of the P. acnes bacteriophage comprises a nucleotide sequence of SEQ ID NO: 1, and wherein the anti-acne compound is sulfur.

91. The composition of claim 77, wherein the genome of the P. acnes bacteriophage comprises a nucleotide sequence of SEQ ID NO: 1, and wherein the anti-acne compound is benzoyl peroxide.


SEQUENCE LISTING (28 sequences)

SEQ ID NO: 1 - DNA, 29752 bp, Cutibacterium acnes
  misc_feature 1..29752: note = Propionibacterium acnes bacteriophage
  source 1..29752: mol_type = genomic DNA
agtgaaatacctcccttttgtggttttgtctgtttgtcgactttttgtgttggtggtgagtgttgtgcagcctgagcttcctgagtctcgtgagtggtgtggggagacgcgtcgttggtggcgtgtgtggggtgaggatagtcgcgcgccgtatgtgtctgatgaggagtggttgtttcttatggatgctgcggtgattcatgattgtgtgtggcgtgagggtcgcgcggatttggtggcttcgcttcgtgcgcatgtgaaggcttttatgggcatgttggataggtattcggttgatgtggcgtctggtggccgtggtgggggttctgctgtggcgatgattgaccggtataggaagcgtaggggggcttgagtaggtgtctggtgttgttgggtctcaggttcctcgtcaccgtgtggctgcggcgtattcggtgtctgctgggggtgatgctggggagcttggtcgtgcgtatgggttgacgcctgatccgtggcagcagcaggtgttggatgattggctggctgtcggtagcaatggcaggcttgcttctggtgtgtgtggggtgtttgttccgcggcagaatggcaagaatgctattttggagattgtggagttgtttaaggcgactattcagggtcgccgtattttgcatacggctcacgagttgaagtcggctcgtaaggcgtttatgcggttgaggtcgttttttgagaatgagcggcagtttcctgacttgtatcgtatggtgaagtcgattcgtgcgacgaatggtcaggaggctattgtgttgcatcatccggattgtgccacttttgagaagaagtgtggctgcagcggttggggttcggttgagtttgtggctcgtagccggggttcggctcgcgggtttacggttgatgatttggtgtgtgatgaggctcaggagttgtcggatgagcagttggaggctttgcttcctacggtaagtgctgccccgtctggtgatccgcagcagattttccttggtacgccgcctgggccgttggctgatggttctgtggtgttgcgtttgcgtgggcaggcgcttggtggcggtaaaaggtttgcgtggacggagttttcgattcctgacgagtctgatccggatgatgtgtcgcggcagtggcggaagttggcgggggatacgaatccggcgttggggcgtcgcctgaattttgggaccgtaagcgatgagcatgagtcgatgtctgctgccggttttgctcgggagcggcttggctggtgggatcgtggccagtctgctgcgtctgtggttcctgctgataagtgggctcagtctgcggtggatgaggcgagtctggttggcgggaaagtgtttggtgtctcgttttctcgttctggggatcgggttgctttggcgggtgccggcaagactgatgctggggttcatgttgaggttattgatgggctgtcgggaacgattgttgatggtgtgggccggttggctgactggttggcggttcgttggggtgatactgaccggatcatggttgccgggtctggtgcggtgttgttgcagaaggcgttgacggatcgtggtattccgggccgtggcgtggtggttgctgatactggcgtttatgtggaggcttgtcaggcgtttcttgagggtgtcaggtcgggtgtgatcagtcatcctcgtgctgattctcgccgtgacatgttggatattgctgtgaggtcggctgtgcagaagcgtaaggggtctgcgtggggttggggttcctcgtttaaggatggttctgaggttcctttggaggctgtgtctttggcgtttttgggggctaaacgtgttcgtcgtggccgtcgggagcgtagtggtaggaagcgggtgtctgtggtatgaactcggatgagttggctctgattgagggcatgtacgatcgtatccaaaggttgtcttcgtggcattgttgtattgagggctactatgagggctctaatcgggtgcgtgaccttggtgtggctattccgccggagttgcagcgtgtgcagactgtggtgtcgtggcctggtatagctgtggatgctttggaggagcgtctggattggcttggctggactaatggtgacggctacggccttgatggtgtgtatgctgcgaatcggcttgctacggcgtcgtgtgatgtgcatttggatgcgctgatttttgggttgtcgtttgttgcgatcattcctcatggtgatggtacggtgtcggttcgtccgcagtcaccaaagaattgtacgggcaagttttcggctgacgggtctcgtttggatgcgggtttggtggtgcagcagacgtgtgatcctgaggttgttgaggctgagcttttgcttcctgatgtgattgttcaggtggagcggcggggttcgcgtgaatgggttgaggtggatcgtataccgaatgtgttgggtgcggttccgttggtgcctattgtgaatcgtcgccgtacttctaggattgatggccgttcggagattacgaggtctattagggcttacacggatgaggctgtgcgcacactgttggggcagtctgtgaatcgtgatttttatgcgtatcctcagcgttgggtgactggcgtgagcgcggatgagttttcgcagcctggctgggtcctgtcgatggcttctgtgtgggctgtggataaggatgatgacggtgacactccgaatgtggggtcgtttcctgtcaatagtcctacaccgtattcggatcagatgagactgttggcgcagttgactgcgggtgaggcggctgttccggaacgctatttcgggtttatcacgtctaacccacctagtggggaggctttggctgccgaggaatctcggcttgtgaagcgtgctgagcggcgtcaaacgtcgtttggtcagggttggctgtcggttggttttttggctgccaaggcgttggattctcgtgttgatgaggccgatttttttggtgatgttggtttgcgttggcgtgatgcttcgacgcctacccgggcggctacggctgatgctgtgacgaagcttgttggtgccggtattttgcctgctgattctcgtacggtgttggagatgttggggcttgatgatgtgcaggttgaggctgtgatgcgtcatcgtgctgagtcgtctgacccgttggcggtgcttgctggggctatatcgcgtcaaactaacgaggtatgataggcgatggcttcgggggttgaggcgaggcttgcggcgactgagtatcagcgtgaggcggtcaggtttgctgggaagtatgcgggctattattctgagcttggtcgtttgtggcgtgccggcaggatgagtgacacgcagtatgtgcgtttgtgtgtggagttggagcgtgccggccatgatggttcggcatcgttggctgccaggtttgtgtcggattttcgccggttgaatggtgtggatccgggtttgattgtgtatgacgagtttgatgctgcggcggctttggctaggtctatttcgaccacgaagattcttgagagtgacccggatagggcgaatgacacgattgatgcgatggcggcgggttttgatcgggctgttatgaatgctggccgtgacacggttgagtggtctgcgggtgcgcagggtaggtcgtggcgtcgggtgacggatggtgatccgtgtgctttttgtgccatgttggctacgaggtcggattatacgacaaaagagagggcacttactactggacatactcggcgtcataagcgtggtggtaagcgtccgtttggttcgaagtatcatgatcattgtggttgtacggtggttgaggttgttggcccttgggaaccaaatagggctgatgccgagtatcagaggacgtatgagaaggcctgtgagtgggttgatgatcatgggttgcagcaatcgcctggcaatattttgaaggctatgcgtactgttggcgacatgagataatttgatgtggtttccggttgtgcgccgccggttattggtgcacagggttgtctcccgcacgggggtcaacaatattgtgttgttttccgcaaggagtgtagggttaggctatggccgatcagagtgttgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggctgaggcccgtaagtgggagtctcgtgctaaaagtaattttgccgagttggagaagcttcgcgcctcggatggtgatgcggggtctacgattgatgagcttcgccgcaagaatgaggaactcgaagaccggatcaatgggtttgttcttgagggtgtgaagcgcgaggtggctgccgagtgtggcctgtcgggtgatgctgtcgctttcttgtcgggtggcgataaggagtcgcttgccgagtctgcgaaagctttgaagggtttgatcgaccatagtagtggtggcgcgggtgtgcgccgtcttgcggggagtgcccccgttgatgatgttaaacgacgtgagggtgtcgcgtttgtggatgctcttgtcaataattctaggagatgatttgtgatggctgacgattttctttctgcagggaagcttgagcttcctggttctatgattggtgcggttcgtgaccgtgctatcgattctggtgttttggcgaagctttcgccggagcagccgactattttcgggcctgtgaagggtgccgtgtttagtggtgttcctcgcgccaagattgttggtgagggcgaggttaagccttccgcgtctgttgatgtttcggcgtttactgcgcagcctatcaaggttgtgactcagcagcgtgtctcggatgagtttatgtgggctgatgctgattaccgtctgggtgtgcttcaggatctgatttccccggctcttggtgcttcgattggtcgcgccgtggatctgattgctttccatggtattgatcctgccactggtaaagcggcttccgctgtgcatacttcgctgaataagacgaagaatattgttgatgccacggattctgctacggctgatcttgttaaggctgtcggcctgattgctggtgctggtttgcaggttcctaacggggttgctttggatccggcgttctcgtttgcgctgtctactgaggtgtatccgaaggggtctccgcttgccggtcagcctatgtatcctgccgccgggtttgccggtttggataattggcgcgggctgaatgttggtgcttcttcgactgtttctggcgccccggagatgtcgcctgcctctggcgttaaggctattgttggtgatttctctcgtgttcattggggtttccagcgtaacttcccgatcgagcttatcgagtatggtgacccggatcagactgggcgtgacttgaagggccataatgaggttatggttcgtgccgaggctgtcctgtatgttgcgattgagtcgcttgattcgtttgctgttgtgaaggagaaggctgccccgaagcctaatccgccggccgagaactgattcatttgttgcggtgatgttttctatgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatacaaggatgccgctaaggctattctgcgcagggccctgttgcgctggaatgataccggggtttcgggtcaggtgcagtacgagtctgcgggcccgtttgctcagactacacggtcgaatactcccacgaatttgttgtggccttctgagattgccgcgttgaagaagttgtgtgagggtgatggtggggctggtaaagcgttcactattacaccgaccatgaggagtagtgtgaatcattctgaggtgtgttccacggtgtggggtgagggttgctcgtgcggatctgatattaacggctatgctggccctttgtgggagatatgatatgaccggttttccttacggtgaaacggttgtgatgcttcaaccgactgttcgtgtcgatgatcttggcgacaaggtggaagactggtctaagcctgtcgagactgtgtaccataacgtggccatctatgcttccgtttcgcaggaggatgaggctgccggccgtgactctgactatgagcattggtcgatgcttttcaagcagcctgttgtgggtgccggttatcgttgccggtggcgtattcggggtgtggtttgggaggcggacgggtctcctatcgtgtggcatcatccgatgtctggttgggatgctggtacgcaggttaatgtgaagcgtaagaagggctgatgggttgtggctcaggatgtgaatgtgaagctgaacttgccgggtattcgtgaggtgttgaagtcttctggggtgcagtcgatgttggctgagcgtggcgagcgggtgaggcgtgcggcttcggcgaatgttggcggtaatgcttttgatagggcccaataccgtagtggtttgtcgtcggaggtgcaggttcaccgtgtggaggctgtggcgaggattggcaccacctataagggtgggaagcgtattgaggcgaagcatggcacgttggcgaggtcgattggggctgcgtcgtgatcgtttacggtgatccgcgtgtgtgggctaaacgtgtgctcaaggatgatggctggctgtccgatataccctgtgtggggacggtgcctgacgatttcagcggtgacctgatttggttggcgttggatggcggcccacagttgcatgttcgcgagcaggtgtttttgcgggtgaacgtgttttctgatatgcctgatcgtgccatgtcgctagccaggcgggttgaggctgtccttgtagacggtgtggacggtgacccggtggtgttttgtcgacggtctactggccctgatttgctggttgatggtgcacgttttgatgtgtattcgctgtttgagctgatatgcaggcctgtcgaatccgagtaaacgttttgttttgatattgttgtttgttttttgtttgatattgtttttgggggttatgatggctggaacacgtaaagcgtctaatgttcgttccgcggttacgggtgacgtctatattggtaaagctcatgccggtgacactattgatggtgtgaagacggttcctgacgggcttacagctttagggtatctgtctgatgacgggtttaagattaaaccggagcgtaaaacggatgatttgaaggcttggcagaatgcggatgttgttcgcactgtggctacggaatcgtctatcgagatttctttccagctgatcgagtctaagaaggaggttatcgagctgttttggcagtcgaaggttactgccggagccgattcgggttcgttcgatatttctcctggtgccacgacgggtgttcatgccctgttgatggatattgttgatggcgatcaggttattcgctactatttccctgaggttgagttgatcgatcgtgacgagattaagggtaagaatggcgaggtgtatgggtatggtgtgacgttgaaggcgtatcctgcccagattaataagaagggtgatgcggtgtctggtcgggggtggatgacggctttaaaagctgatactcctccgactcctcctccggccccgaatcctccgaagcctgagccggatccgaatccgccgtctaataactgatacacatagtttgagggattgttgatagatgagtgacacgggttacacgttgaagattggtgaccgtagctgggtgttggcggatgcggaggagacggctcaggctgttcctgcccgcgttttccgtcgtgctgctaagattgcccagtcgggtgagtctgcggatttcgcccaggttgaggtgatgttttctatgttggaggctgccgccccggctgacgcggtggaggccctggaggggcttcctatggttcgtgtggccgagattttccgccagtggatggaatacaagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccacctgaatggttggaagcgcccgtttgagtggtgcgagtgggctgtgttggacatgctggatcattacaggtctgctaatagtgaggggcagccggagcctgtggcgaggcctacggatgagcgtagggcccggtttacgtctgggcaggtggacgatattttggcgcgtgttcgtgctggtggcggggtgtctcgcgagattaatattatggggtgaatagtgtatgtctggtgagattgcttccgcatatgtgtcgttgtatacgaagatgcctggtttgaaggcggatgttggtaaacagctttctggggtgatgcctgctgagggtcagcgttcgggtagtttgtttgctaagggaatgaagttggctcttggtggtgcggcgatgatgggtgccatcaatgttgctaagaagggcctcaagtcgatttatgatgtgactattggtggcggtattgctagggcgatggctattgatgaggctcaggctaagttgactggtttgggtcatacgtcttctgacacgtcttcgattatgaattcggctattgaggctgttactggtacgtcgtatgcgttgggggatgcggcgtctacggctgcggcgttgtctgcttcgggtgtgaagtctggcgggcagatgacggatgtgttgaagactgtcgccgatgtgtcttatatttcgggtaagtcgtttcaggatacgggcgctatttttacgtctgtgatggctcgcggtaagttgcagggcgatgacatgttgcagcttactatggcgggtgttcctgtcctgtctttgcttgccaggcagactggtaaaacgtctgctgaggtgtcgcagatggtgtcaaaggggcagattgattttaacacgtttgcggctgcgatgaagcttggcatgggtggtgctgcgcaggcgtctggtaagacgtttgagggcgctatgaagaatgttaagggcgccctgggttatcttggtgctacggctatggccccgtttcttaacgggttgcggcagatttttgttgcgttgaatccggttatcaagtctgtcacggattccgtgaagccgatgtttgctgccgtcgatgctggtattcagcgtatgatgccgtctattttggcgtggattaaccgtatgccggctatgatcactcgaatgaatgcacagatgcgcgccaaggtggagcagttgaagggcgtttttgcaaggttgcatttgcctgttcctaaggtgaatttgggtgccatgtttgctggcggcaccgcagtgttcggtattgttgctgcgggtgttgggaagcttgtcgcggggtttgccccgttggcggtgtcgttgaagaatctgttgccgtcgtttggtgctttgaggggtgccgccggggggcttggtggcgtgtttcgcgccttgggtggccctgttggtattgtgatcggcttgtttgctgccatgtttgctacgaacgcccagttccgtgccgctgttatgcagcttgtgggggtggttggccgggctttggggcagattatggtcgccttgcagccattgttcgggattgttgctggcgtggttgccaggttggctcccgtttttggccagattattggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtggtcatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgccgtgctacaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatatcaaatcgtttacgtcggcgtttattaacggtttcaagtcgatcatttctgccggtgttgccgcggttgtggggttttttacgcggcttggtttgtcggttgcctcccatgtgaggtctggttttaacgcggcccgtggtgctgtttcttctgcgatgaatgctattcggagtgttgtgtcttcggtggcgtctgctgttggcgggtttttcgggtcgatggcgtctagggttcgtagtggtgctgtgcgcgggtttaatggtgcccggagtgcggcttcttctgctatgcatgctatggggtctgcggtgtctaacggtgtgcatggtgtgctggggtttttccggaatttgcctggcaatattaggggcgccttgggtagtatggggtccctgttggtgtcggctggccgtgatgtggtgtctggtttgggtaacggtatccggaatgctttgagtggcctgttggatacggtgcgtaacatgggttcccagattgcgaacgcggcgaagtctgcgctgggtattcattccccgtctcgggtgtttcgtgacgaggttggccgtcaggttgttgccggtttggctgaggggatcaccgggaatgctggtttggcgttggatgcgatgtctggtgtggctggccgtcttccggatgctgtggatgcccggtttggtgtgcgatcgtctgtgggctcgtttaccccgtacgaccggtatcggcgtgcgaacgagaagagtgttgtggtgaatgtgaacggacccacgtatggggatcctgccgagtttgcgaagcggattgagcgtcagcagcgtgacgctttgaatgcgttggcttacgtgtgatcgagggggtgttgtgcatgtttattcctgacccgtctgatcgtgccggtttgactgtggattggactatgtttccgttggtgggtaatgctccggagcgtgtgcttcatttgacggattatacggggtcgtctccggtcatgttgttgaatgattcgttgcgcggcctgggtatgcctgaggtggagcagttttctcaaacgcatgttggtgtgcatggttcggagtggcgcgggtttaatgtgaagcctcgcgaggtgactttgccggtgttggtgtcgggtgttgacccggatccggtgggcgggtttcgtgacggttttttgaaggcgtatgacgcgttgtggtctgcgtttcctccgggcgaggtgggggagttgtctgtgaagactcctgccggtcgtgagcgtgtgttgaagtgccggtttgattcggctgatgacacgtttacggttgatccggtgaaccgtggctatgcgcgctatctgttgcatttgacagcttatgatccgttttggtatggggatgagcaaaagtttcgttttagtaacgcgaagttgcaggattggttgggtggcggccctgtcggcaagaagggtaccgcgtttcctgtggtgttaacaccgggtgtgggctcgggctgggataacctgtctaataagggtgatgtgcctgcgtggcctgtgattcgtgttgagggtcctttggagtcgtggtctgtgcagattgatggtttgcgtgtgtcttcggactatccggtcgaggagtttgattggatcactattgatacggatcctcgccagcagtctgcgttgttgaacgggtttgaggatgtgatggatcgtttgacagagtgggagtttgcgcctatcccgcctggcggttctaagagtgtgaatattgagatggttggtttgggtgctattgttgtgtcggtgcagtacaggtttttgagggcttggtgaatagttgatggctggtcttgttccgcatgtaacattgtttacacctgattatcgccgtgtggcgcctatcaatttttttgagtcgttgaagttgtcgttgaagtggaatggtttgtcgactttggagttggtggtgtcgggggatcattcgaggcttgacgggttgacgaagccgggtgcgcggctggttgttgattatggtggtggccagattttttctgggcctgtgcgtaaagtgcatggtgtgggtccgtggcgttcttcccgtgtgactataacgtgtgaggatgatattcggctgttgtggcgtatgttgatgtggcctgtgaattatcgtcctggtttggttggtatggagtggcgtgcggacagggattatgcccactattcgggtgcggctgagtcggttgctaagcaggtgttgggggataatgcttggcgttttccgcctggtttgtttatgaacgatgatgagagtcgtggccgctatattaaggattttcaggtgcggtttcacgtgtttgccgataagttgttgccggtgttgtcgtgggctcggatgactgtcacggtgaaccagtttgagaatgcgaagtttgatcagcgtggtttgttgtttgattgtgtgcctgctgtgacccggacgcatgtgttgactgccgagtctggttcgattgtgtcgtgggagtatgtgcgtgacgccccgaaggctacttcggtggtggttggtggccgcggcgagggcaaagatcggctgttttgcgaggatgttgattcgatggccgaggatgactggtttgatcgtgtcgaggtgtttaaggatgcccgtaacacggattccgagaatgtgcatcttattgatgaggctgagcgggtgttgtccgagtcgggggctacgtcggggtttaagatcgagttggctgagtcggatgtgttgcggtttgggcctggccgcctgatgccgggtgatcttatctatgtggatgtgggctcggggcctattgcggagattgtgcgccagattgatgtggagtgtgattcgcctggtgatgggtggacgaaggtgactccggttgctggggattatgaggataatccgtcggcgctgttggctcgccgtgtggctggtttggctgcgggtgtgcgggatttgcaaaaattctaattgttaggggtttgttgtgggtattgtgtgtaaagggtttgatggtgtgttgaccgagtatgattgggctcaaatgtctggtctgatgggtaatatgccgtccgtgaaagggccggatgattttcgtgtcggcactacgattcagggttccacggtgttgtgtgaggtcctgccggggcaggcttgggctcacggggtgatgtgcacgtcgaatgctgttgagacggtgacaggtcagcttccgggcccgggtgagacccgctacgactatgttgtcctgtcgcgggattggcaggagaatacggccaagttggagattgttcctggggggcgtgcggagcgtgcccgtgacgtgttgcgtgcggagcctggcgtgtaccatcagcagttgttggctactttggtggtgtcgtctaacgggttgcagcagcagcttgacaggagggctatagcggcccgtgtggcgtttggggagtctactgcatgtgatcctacccctgtggagggtgaccgggtgatggtgccttctggggctgtgttggctaatcatgctaacgagtggatgctgttgtctccgcggattgagacgggcactaagtcgatcatgtttggcgggtctgctgtgtatgcttacacgattccgtttgatcgccagtttgctagtccgcctgttgtggtggcgtctatggctacggcggctgggggcacgacccagattgatgtgaaagcctacaatgtgactgcccaaaattttagtttggcgtttattacgaatgatggttcgaagccgaatggtgtgcctgcggtggctaattggattgctgtcggcgtgtgactgtacaggtgttgtggcggatggtgtgatgttggggggctgtggtgtcgtggtttactcctgcactggtggcctctatttgtaccgcgttggccacggttttgggttctgttcaggctgtcacgtctaaatctaggaggcgtttgcgccgcctgtcggcgcaggtggatgcgatggaagagtatacgtggggtgtgcggcgcgaggtgcgaaggtttaacgccgggcttcctgacgaggtggagcctatgcatctccctgatttgcccgagtttttgaaagatactgttgatggtggaggtgagtagggttgagggagttggaggaggagaagcggcagcgccgcaattttgagaaggcttcactggtgttgctgtttttgtcgcttgtgttattggctgtggttgctgcgggtgctttgcgtttcggggctgtatcctctgagcgggattcggagcaggcgagggcccagtcgaatggtacagccgccaagggtttagccagcagtgtgcggcaggtgtgtgctcagggtggacgggagtctgtgcggcttcaccagtctggtttgtgtgtggatgctcagcgtgttgagcgtagtgtgcagggtgtgccgggtcctgccggtgagcgcggcccgcaaggcccggcaggtgtggacggccgggatggtgttaatggttcggctgggctggttggccctgtgggtccgcaggggtccccgggtttgaatggtgtgaaaggtcctgacgggttgcctggcgctaacggttcggatggccgtgatggtgtggacggtgtgaacggcaatgatggcgctgatggtcgggatggttcggccggtgagcgcggtgatgtgggcccctcaggtcctgccggcccgcaaggtgcacagggtgaacggggtgagcgcggccccgccggtgcgaatggcacgaatggcaaggacggtaaggatggtgccgacggccgtgatgggcgttcggttgtgtctgtgtactgtttcggtggcctgccagggtgtgaaaccatcacctgtggttaccgtgtcatcccgtaaatagaagaagagggaagggtgttactagtgttgattgtggtttttggtggtggtgtgtggtgagatacattcctgcagcgcatcactctgccggctctaataatccggtgaacagggttgtgattcatgcaacatgcccggatgtggggtttccgtccgcctcacgtaaggggcgggcggtgtctacagcaaactatttcgcttccccatcgtctggtggttcggcgcattatgtgtgtgatattggggagacggtgcaatgcttgtcggagtctacgattggttggcatgccccgccgaatccgcattctttgggtatcgagatttgcgcggatgggggttcgcatgcctcgttccgtgtgccggggcatgcttacactcgggagcagtggcttgatccgcaggtgtggcctgccgttgagagggcggcggtgctgtgtagacgtttgtgtgacaaatataatgttccgaaaaggaaactgtcggctgccgatttgaaggctggcaggcggggtgtgtgtggccatgtggatgttacggatgcgtggcatcagtcggatcatgacgatcctgggccgtggtttccgtgggacaaatttatggccgtcgtcaacggcggcagtggagatagtggggagttaactgtggctgatgtgaaagccttgcatgatcagattaaacaattgtctgctcagcttactggttcggtgaataagctgcaccatgatgttggtgtggttcaggttcagaatggtgatttgggtaaacgtgttgatgccttgtcgtgggtgaagaatcctgtgacggggaagctgtggcgcactaaggatgccctgtggagtgtctggtattacgtgttggagtgtcgtagccgtcttgacaggctcgagtctgctgtcaacgatttgaaaaagtgatggtggtttgttgtgggtaaacagttttggttaggtttgctagagcgggcggctaagacttttgtgcaaacgtttgttgctgtgttgggggtgacggcgggtgtcacgtatacggcggagtcgtttcgtggtttgccgtgggagtctgcgttgattacggctacggttgctgcggtcctgtcggtggctacctcgtttggtagcccgtcgtttgtggctggtaagccgaaaaccacgcctgtggatgcgggtttggttccgccggatgatcccggaatagtggagcctcacatggtggatgtgtcggatcctggcatgatcgagcctgcagatgatgtggatcttggtgtaggctatgtgccgaaacatgctgccgagtcggaggttggcacggtagagtcgactgttgcataagtgaatatagatgtgtgccccagcggtgctgccacgattgtgtggtggttgccgctggggcactatttttgtatattgcggtgtggctatgattcgttgctgtcgatggtgtcttcgagcatctggtacaggtggaggcaggtagagatagtttcgctggcctggtcgagaacgttccggccgataacatttttgttgttgtcgcggtggcggatgatagaccacatgatctcgtcggctgccgcctgcaatagttttgcctggtatgcgattccagcgagccagtctagtgcttcctggcttgcatagggtgtctggtcctcgctgttgcttgtggggtgtcctgcactgtcgcatagccacaggatttcgctgcactcgtctagcgtgtcctggtctatagcgagatcgtcgaggctgacattgttgacggtaaggttcacgttgtcgagggagatgggtacaccgtactggttttcgacaccgtcaacaatgttttccaattgctgcatgttggtgggctgttgttggacgatacggtgtatcgctgtgttgagggtggtgtaggtgatattgtgtgtgttgttcatcgtgttatgccattccttcgttatcgtctggcctgtagtatgtgctgtttgcgtactcggttaacgtcatcagtgtttggtctgcccactgtttcacagtctgccttgtcactccgagtcgttgggcggctgtggcgtaggtttggtcatacccgtatacttccctgaatgctgccaaccgtgccaaatgttttcgctgtttggatggctggcaggcgagggtgtagtcgtcgatggctagctgtagatcgatcatggtggcaatgttgttgccgtggtgttgtggcgcggttggtgggggtggcattcctggctccacactgggtttccatgggcctccgttccagatccattgggcggcttggatgatgtctgcggtggtgtaggttcggttcactggtcatcccctgaacaggttgtctgggttgctggtgcggattgtgtcgaatcgtccgacgcagtggcagtagtcgtacatgagtttgataatgtgttggtggtctcccaaataggtgtttccgctgatgctgtaggtggctgtgccgtctttactaatagtgtatttggcggtgatggtttcggggttttcggtgtcggtgatgatggctgtggtggtggtgcctacggtttggagcacggtggtttgggttccgtcgtcgatggtggttttaaccatgaggtgtgttctccctttgtgttagttgctggtttggttgtcggctagatgaatgatgtcgggtaagggtttcggctggtctaaatgttgtgtggttttgttggctagccgtttggctaccctgtagcacattttggtgtagtgtttgttgtctaggttgtggtattgttcccgcaccgcaatatatagcagggagtcttggtacaggtcgtctgcattgattgcggggtagtgtgcggctgttttagtgcatgcccggttgagtgtgcgtagatgatggtctgtggcccacacccacgatgcggtggtggctaggtcggcttttgttggtcgtcggctcatggcatctctttcatctggctatctggtagttgtttggtgttttgttgttgatagtgtagcacacgagtccggggtttccggtggtgcccgtcttgtgccggtaccatgtggattcgccttccatggatgggcattggatgaaggtgcgttgtccttgttcggagatttctaggtggtgcctgtgtccggccatgaggatgtgggatgtggtgccgttgtggaattcttgtccgcgccaccaatcatagtgtttgccggtgcgccattggtggccgtgggcgtgtagtatccgtgtgccggctacttcgacggtggtggtcatttcgtctcggctggggaaataaaagtgtaggttggggtattggttggtgagctggtaggcttctgcgatggcgcggcagcagtctacgtcgaaggagtcgtcgtaggtggtgactcctttgccgaagcgtacggcttctccgtggttgccggggatggatgtgatggtcacgtttttgcagtggtcgaacatgtggatgagttgcatcatggccatgcgggtgagcctgatttgttccgtcaagggggtttgtgtgcgccaggcgttgttgcctccttgtgacacgtatccttcgatcatgtcgccgaggaatgcgatgtggactcgttcgggtttgcctgcctgctgccagtagtgtttagctgatgtgagggagcgcaggtagtcgtcggcgaagtgtgatgtttccccgccggggatgcctttgccgatttggaagtcgcctgccccgatgacgaaggccgcagtgctgtagtcggtgcgggtgtcctgttcgggttttgggggtgtccattcggctagtttatcgacgagttcgtctacagggtaggggtttgttgcgggttggtggtcgatgattttttgtacggatctgcctgtttctccgttggggagtgtccattcggagatgcgtgtgcggcgtacggtgccgtttgcgagatcatcgcagatggtgtctgcttcgctatcgtggttggctagctgggtgagtagccggtctatgttgtctatcactgggtatcctcttcttgcggggtggtgttggcttgtttgcggcggtagtcttttataacggtggcggagatggggtatcctgcctgggtgagctgttttgctagccatgaggcggggatggttttgtcggcgagcacgtcggcagccttgttgccgtagcgttggatgagtgtttcagttttggttgccatggtgtcctatcggttgtgtggtgggctgccatcctgtgcggcagtcgccgtcgtggcctggtttgcgtgtgcaccacgatacggttctgtctgtgtggttgagtgttttgccgcacatgacgttttgtagatgctctggcagtgcgccgtcaccctggttgctggtttgtgtgtcgaagagtgttttctggttggtgaaatgctcggacacggtgccattatgtacgggtagtatccatgttttccattgttgttgtagccgggtgttccagtggaattgttttgctgcgttcgtggcttgtttgatggttttgtagtagccgacgaggatgcgctggtgttcactgtcgggagggttttggcctcgccagtattgtgccgccacggcgtagcggttgctggctgtgaaggcgtcccagcagtattcaataatgtgttgtagtacactatcgggcatgtctcgtacttggttttcgtcgagccacgcgtcgacaatgatgttgcgtatggcgcgtttgtctttggtggtgggtttgaatgcgatgctcacagtacgggcctgtcgtcttgcatgaaatcattaaaggatgattcgcttgcgcggcgtgcttgtgtgatttgctggtcagaccagtcggggtgttgctgtttcagatagtaccagtggcacgcattgtaggtttcgtcttgtagccgggtgagatggttttcggtgatgatttgtttccacatagtccatgacacgtcgagccggtccaatatttccattgctggaatgttgaactggttcaggaagagtatttcgtgggtgtagtattccttctcgtactggtcccatccacttcggtgcctgttgggctggtttttggggtaggcttcccggcatactttgtgcaaatgtttggccatgtcgtcgggtagtttaatgtcagggttggcgcggatcatggatcgcatcccatcataggtggtgccccaggtgtgcatgatgtaggtggggtcttcaccatcagcccatttttctgcacagatggcgaggcggatgcgtctcctggctgattggctggtgttgcgccggttggggatggggcacgtgtcgaggggatccatgatgttttggtgtacctttcttggtttaggttgcttgtgtggttttattgtagcactgtgtctagtgcttgtgtcaaccctgttttgccggcctgaaggtaggtgtctgtgacatcccccagggtgaggggcacatgggtggcttgggggagtgcggcctggagtgtttgggccatctggtggcccgccttgtctgggtctgaccagatgtagatgtggtcgtagccttcaaaaaatttggtccaaaaagtttgccacgaggttgcgccgggtagggctacggctggccatccgcattgttcgaggatcatggagtcgaattcgccttcgcaaatgtgcatttcggctgccgggttggccatggcggccatgttgtagatggagcctgtgtctcctgccggggttagatatttggggtggttgtgggttttgcaatcatgttggagtgagcagcggaaacgcatttttcgtatttcggctggcccttcccagacggggtacatgtatgggatggtgatgcactggttgtagttttcgtggccttggatggggtcattgtcgatgtatccaaggtggtggtagcgggctgtttcttcgctgatgcctcttgccgagagcaggtcgagtatgttttcgaggtgggtttcgtagcgggctgaggctttctggattcggcggcgttccgcaatgttgtaggggcgtatgctgtcgtacattcgggttttcttcctctaatcgttgtttcagtttgtggagtccgcctccgataccgcatgtgtggcagtaccagacgcccttgtcgaggttgatgctcatggagggctggtggtcgtcgtggaacgggcagaggatgtgttgctcgttccgtgacgggttgtagcgtatctggtgggcgtctaggaggcggcaggtgtcagaggtgtgggaggagctcgttgagggttgataccacataggcttcgctccagggtttgttgcgctgtttcatgatgacgagtccgatggtggattggttttcgcggtttcggtgtgtttcgtagttgcgtgcctcccggctggcttgtttcacgaattcggctaggtgtgcctgtcctgctttggcttcgatcacataggttttgttgccggttgtgaggatgaggtcgccttcgtcttctttaccgttgaggtggaggcgttctatatcatagccggtgtcgcgtagctggtggaggagtcttgtttcccattcggcgccggctcggcggttgcgtgcctgttgtgttgacatgatagtcctttatgttcttgtgtcatgttccagggctgtttttctactaggggcccgaagaatgtgtattcggggtaggctcgtagtcgttcgtattttgttccgtctgggctggatttgccggttctctgtttcaggacggcgatgcgtgcctcggcggggatggtgaggccgttgccgttgtcttcgccaccatacagggagactcccaatatgagttgtggtttttcggagaggccgtttttgatttcccgcctagctggggggtgttcgatgtcggtgccggttttgtcggttgcgtggtgggtgacgatgatggtggagccagtatctctacctaaggctgtgatccattgcatggcttcttgctgtgcctgatagtcggattcgcagtcttggatgtccatcaggttgtctataacaataatgggtgggaaggtgttccacatttccatgtaggcttgcagttccatggtgatgtctgtccatgtgatgggtgactggaatgagaaggtgatgtgtccgccgtggtggatgctgtctcgatagtattctggcccgtagttgtcgatgttgtgttgtatctgttgggtggtgtgttgggtgttgagtgagatgattcgtgtggaggcctcccagggtgtcatgtcccctgatatgtagagggctggctggttgagcatcgcggtgatgaacatggctagccctgatttttggctgccggaccgccccgcgatcatgaccaaatcccctttgtggatgtgcatgtccaggttgtcatacaagggtgctagttggggtatgcggggcagttcggcggctgtttgggaggccctctcgaaggatctttggagagagagcatcgggaccttaatctatctgttggttgggtgtgttttggtggtcagatggagtcgatgtcgatgtcagcatcggcgggggctgtggtgtcgtctagctggccgttgtcgcgtttgtctacatattcggcaaccttatcgtagatggcgtcgtcgaggggtttgaggacgaccgcgttgaacccgtttttggtgcgcacggtggcaagtttgaaggcttgttcttcgccgagatatgcttctaggtcgcggatcatggagtgtgggcggtcgttgttgccgcgtgctttttcgatgatggcgttggggatggtttctggggtgccgttgttgagatcctggagggtgtggaagattgtgacatcagcgtagatgcggtctgcgacctgtccaccgtagccttcggtgttgtgttctacgtcgcggattttgaaggcgatggcggtggcgtcctggtttcgggaggggttgaagaaggtgctgttgctgttgttgtggtagttggcgagtgccatgattgtgttatcctttactgttgtgtctgtttttgttgtcttatattggtttatcgggtgaggctgtttcgtttgctgcggaaagcctcggaaacgtcactgttactggtgatggtcttcttgtactgtttgagtaggtctgctagctgtgtcttgctggtggctttgtttatccggtcgatgatgatgtcgttttcctgtgatgcgattttgttgacgtagtctttggcggctttatcgtatcggtcttgaagcaggattgctgcgctagcgatgagggttgcgagatcccagtctttggatacggtttcgtctttcaatcctcctagcagatcaataatggattgtttgatgtcttctgcggtgtctccgcggatgactgtccatggggcggcatagtcgccaccgtatttgagtgtgatagttagttttccgctgtctgtggtgtgctcgtcggtcacgtgttttccttttcgttgttttcggcttctggtggctgtacggtggtttctatcgggtatctgtaggcgtctttcccgttgacggcccagcaggcgtccttgacggggcatcctttgcagagtgtggtgacgtggggtacgaagatgccttggctgattcctttcattgcttgactgtacatggatgatacatgccggtaggtgttgttgtcaagatcaatgagttcggttgctgtgccctgctcgactgattgctcgtctcccttggtggtggcgggtgtccaaaacatgcctttcgtcacatggatgccgtgttgggcgagcatgtaccggtatgtgtgcagctgcatactgtctgcgggtaggcgtccggttttgaggtccaaaatgaaggtttcgccggtgtcggtgtcggtgaatacccggtcaatatatccgactatttttgtgtcatcgtcgagggtggtttctaccgggtattcgatgcctggctggccgtcaataacagcggtggcgtattctgggtggttgcgcctccatgttttccagcggtccacaaaggtggggccgtacatcatccaccaattgtagtctttcttgtgtggcccgcctgactcgcacatgtttttgcatattctgccggagggctttatgtttgtgccttcggattcggcgagggcgatttgggtgtcgaaaatgtttgtgaaggatgagagtttgtctggcagtgcagggtattcggcggggttgtacaggtgtaggtcgtattgttcggtgatgtggtgtatggcgcttccggcgatggtggcgtaccaggtgtggtgttgggcgtggtagccgtgtgctaggcgccatttttcgccgcattcggcccactgtgtgagtgaactgtaggagatgtggcctggatggttgatggttttcgggtattgtgctaggggcattacttgtcgcctttgtgggtgttccatgggttgcgggtgtctttgccggcgtggtgttgctggtaggcgaggagtgcgaggcagtgccaggcagcgtgtgccagatgcggcaaatgtgattcgttgtcgaggttgttgccttgctgccatgataacaggtgccggtagagggcgtcgacactgtggctccacgggtatcctccggtccagttgttgtcgccgtacttggtggcaccgtagcctgccacggagcctagggcgtgcaaggctgcggggtcgatgagggagagcctgcagagtttcaattcttttcgggcaccgctgttggggtcggtgtacatgctggtgggctcatccatggtgtgtgtgctccttaagcgtgggttactggttattgtcgtgggcgagtgctacggcgagaataatgatggcgagggtttcagcgatcagtatgggtgttgtgatcatttagtgtctcggggattattggtgagtgttgatgcacctaggagggtggcgagggcgcatgcggcgatggtggcgagggctgccttgtgtggggtgccggttgcgtacatccatgtgatgatgccgccttggatccaggctagactggtgaagaacgtttcgtaactgtgtagctcaatgttgttgttgggtgtgttcatgcttgctcctgaagaatggtgttgatggttttataaatgttgtacaggtcggtttcgatagataacagttggttgatttggtggtcgagatcaatgtctgggttgagggtgtcgatgcgggcggcgatatcggtggcggtgcgtaggcttactgctgcaccgtggatgatgtggcacatgtcggtgaggccgactttggcgatatagtgtgacatgagaggcataataggtgtgctgtctttctggtcagcgtgaagggttgatggacatatcctctacctgtggtttgtcttcggtgccggagacttggcagaagactttcacatgcgtcttggatgctccggcctgtttggcggtggcaccgtaggcgatagtaaaggtgtctttgtgggcgccgatgactttgtgtaggaagaggtcgatgtcggggttgccgttccatttgacaccgttttctgcggctgtctgggtggctttctgattgcaggcgtgtgcggcggtgatcatggtgagacccttgctggtttcttcaccccttgcttgggcttgccggtgggctttggcctgctcggcttgtagggagcggactgctgcggcctggcgggccttcttctcagccttgcgctgctggacggttttgggtgtccattcggtgttggctgtggttacctgtggtgcgggttgtgaggcgagtggcggattgtcgtctggggctggcatgaaggatgctgcggcaataatggcgactgtggcgcctgcgatggtgtagcctgttttcttgttcatgattttatgttcccctttccggggtgttgttcgttgctgacatggttaatactttcagcggctgggcccactgtcaaggctgcgctcagtttgtgtgagcgtttcttgtgtggctaggggtgatggcttctttcgcccaataggatgtgccaccgctggtccagtatccgagtttgttgcgctgcatgcccttggcgtccatctcgtcgatagtgaggcacctgcggcgattggggcctgtcttgaccccgtggtcgcctgtccggtgcatgtcgcctgaggtggtactcgtgaatgtttcatggcagatggtacagtgctctggtcgatatccggtgattgtgctatcgcacttgtggcatgtccattccatgattgctcctattttccattataagacttcctgtagtgccattttagcgccttgcgggtcttgggggtacaactatataggtcaggtgtttctaggcgattctaggctcattgtgtgtggctggggttttatcgggcacacagggtgagcaggtggccaacattgatgcgggtcacattccagtagagttgcgtggcttccccactggtgagcggcttccactcgtcatggctgaacacggtgccatcggatgcgatgaacgtgttggggcgtagcttgtggagttcggcttccacgctctgccggtaggcttcggcgaggccctcaaaatccatgtggtcgcaggggaggttttcgaggcgtgtcaggtcgaagggtgtggggcagtcgtagctggcgggggtgtagagctgggtgaagtggttggcgatcttctgcatcatgattccttttctgatgatggtgtgttgagggtttatcgggtggatgcgacaaggatggcgtctacatcgatcatgtcgatgagatcgtggagttcctcggcctcgttctcagtgagtggctgccaggcgtagtcgccgtatacggcgccgtcgagggtgacagtccacgggggccggatgagtcgtatggcttcttgtactttagcgtggtacatgcggcgcaccatatccagatcgatgtcgtctgaatggtttccggtgaggctgtggaggctgagcgggtcgatgtctgtctgcctgtagagggatgtgaaggatggggtgatgagtgtgccatccatgagtgtgctcctttcggtggttgtaggggttgttgtggtttctagagtgtgcgggctgcgaccccacagtcaaggtgtcgctcaaactcagtgagcgtttcatatgggtgtgttgggtgtgacagatgtcacttaagccttgatggcctctctcagcgcctcaaatcttctaggggtaggattatgaagggttggccctgctgatcgattctaggccccatacagggcgtctgaggggtgtgtctgagtgatagtgggtgtggcagatgatctagcgagtcaaggtgccgagctgagacataagatctatcatctaggtgtgtgagatgtatcacatcctcccggcttggtgtgcaccctcaaggccacccagtcgatctgacgtggagggtgtagcccagaaatactgtttaaagccttcacacggcgcctaggagcgccttacagggtgggggctaggtatttatacccccagcacattctgatcgattctagacgcctacaggagcccgatacacgatcagccatccagacgcagatcatcagcacctatcatggttagctaagcctcaactatgtggacagtgttggttactgtgggggaagaaggacacggtaaaagaaagagggggagtatcagctttaaagccttaaggtcttagcgcttagcaccgatggtcttagcagttagcaccgagccccctcaagggctcggcatcagcccgaacaggcacagccatgaaaggagtacacgccatcagggaaggctttcgagtacgaggagcctcagcgacgagtactcgaaagcctgagggaacacccatcagcactgatgagcctagcgtattcggaaaggacacaagagtgaagtgtgacagctgtccgggagtgaaccccgttctgactaggggtttcagccttaaccaccctcaaaggttacaagactctaagaaaatttaaggaaaagtttaggtttaatttttggacctttactaccaaaaacacccgtttacagccctcaaacccgcctatagagccaaaaccaccagtttgactcatcccaggtggggtatgataggctggacaggtagccagctggacgcaaggccggaaagtgctaacgcactttccaacctcgcttaccatcagtctaccaaacacttaaagacctaagggcttagcgctaaggtgctgatagcttagcaccgagccccctcaagggctcggcatcagtcttaaagccttaaatacttaaagtaactataaaactttaaaagcttaacacttaaggatataaactttacatcagtgtttaagacttaaaaacttaaaataactattaagacttaaagtaactataaaacattaaagaccttaagtacttaaagttaaccatcagtcttaaactttactatgataacctataagtcttaaagcttataggtataataatataatataagtattaaagcttataagttataaaagttttagaagagttaaagggttaacttctttacttctcttctctctttggttctttctctcttctcttcttttcttcatcgggggagaagaggaacctttaacgtcaacgctgatggacttttcgccgtgtgtctcgtgtgcttctggtcgcaagctcccatcgcacactccccacactctttcacctgtgtccctttcaggcttagcgtgttcagctgaaggcgtacagcgtgtcacgcttaaacccttaacaccaggtaagacttaaagtgcatattataagtagaagactttaaaaccttaagggtgttcctgcttagcctgtgtcctttaacgctaggcgctaagccgtgaaacgtgaacacccatccacccctcttctttttaccgtgtccttcttcttttgacaccgctggggggcgatgtgatctttttaacatgccagggggtgcgggtagaaaacaaccaccccaccacaaacagaacaccccctcaaacgcacaaaacagcccccaggatcgatgaacagggcaagggcaaggtattcatacccccagacgattccaggccgttagagaggcaaataagacccgtacagggctaggtgaggaatagacacatcatggcacgcaccaatcgcacagctagccaagcccaccgacgctggcggcaacgactcatcacccaagcccaacaacaaggccaaaccgaatgcccactctgcggagtcaccatcacctgggacacacacgacctaccaaccagccccgaagccgaccacatcacacccgtcagcaggggaggactcaacaccctcgacaacgggcaaatcatctgcagaacatgcaacagaagcaaaggcaatcgcagcgaaccaaacatcaaattccaacaacaaaccacaaaaacattgattccatggtgacaaacccgccaacccccaccggggacaccccctgcacaggcgtgcaagacctcgtacggctt

SEQ ID NO: 2 - DNA, 1537 bp, synthetic construct
  misc_feature 1..1537: note = 16S DNA sequence for the KPA171202 type strain
  source 1..1537: mol_type = other DNA
tttttcattggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaaggag

SEQ ID NO: 3 - DNA, 1537 bp, synthetic construct
  misc_feature 1..1537: note = 16S DNA sequence for the ProI probiotic strain
  source 1..1537: mol_type = other DNA
tttttcattggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgcgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagttggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaaggag

SEQ ID NO: 4 - DNA, 1537 bp, synthetic construct
  misc_feature 1..1537: note = 16S DNA sequence for the ProII probiotic strain
  source 1..1537: mol_type = other DNA
tttttcattggagagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggattgggagcgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaaggag

SEQ ID NO: 5 - DNA, 1525 bp, synthetic construct
  misc_feature 1..1525: note = 16S DNA sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatgcctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcctcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 6 - DNA, 1525 bp, synthetic construct
  misc_feature 1..1525: note = 16S Sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatgcctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 7 - DNA, 1525 bp, synthetic construct
  misc_feature 1..1525: note = 16S Sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtaatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 8 - DNA, 1525 bp, synthetic construct
  misc_feature 1..1525: note = 16S Sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcggaagcgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 9 - DNA, 1525 bp, synthetic construct
  misc_feature 1..1525: note = 16S Sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgtccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatggctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctatgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 10 - DNA, 1525 bp, synthetic construct
  misc_feature 1..1525: note = 16S Sequence
  source 1..1525: mol_type = other DNA
agagtttgatcctggctcaggacgaacgctggcggcgtgcttaacacatgcaagtcgaacggaaaggccctgcttttgtggggtgctcgagtggcgaacgggtgagtaacacgtgagtaacctgcccttgactttgggataacttcaggaaactggggctaataccggataggagctcctgctgcatggtgggggttggaaagtttcggcggttggggatggactcgcggcttatcagcttgttggtggggtagtggcttaccaaggctttgacgggtagccggcctgagagggtgaccggccacattgggactgagatacggcccagactcctacgggaggcagcagtggggaatattgcacaatgggcggaagcctgatgcagcaacgccgcgtgcgggatgacggccttcgggttgtaaaccgctttcgcctgtgacgaagcgtgagtgacggtaatgggtaaagaagcaccggctaactacgtgccagcagccgcggtgatacgtagggtgcgagcgttgcccggatttattgggcgtaaagggctcgtaggtggttgatcgcgtcggaagtgtaatcttggggcttaaccctgagcgtgctttcgatacgggttgacttgaggaaggtaggggagaatggaattcctggtggagcggtggaatgcgcagatatcaggaggaacaccagtggcgaaggcggttctctgggcctttcctgacgctgaggagcgaaagcgtggggagcgaacaggcttagataccctggtagtccacgctgtaaacggtgggtactaggtgtggggtccattccacgggttccgtgccgtagctaacgctttaagtaccccgcctggggagtacggccgcaaggctaaaactcaaaggaattgacggggccccgcacaagcggcggagcatgcggattaattcgatgcaacgcgtagaaccttacctgggtttgacatggatcgggagtgctcagagatgggtgtgcctcttttggggtcggttcacaggtggtgcatgcctgtcgtcagctcgtgtcgtgagatgttgggttaagtcccgcaacgagcgcaacccttgttcactgttgccagcacgttatggtggggactcagtggagaccgccggggtcaactcggaggaaggtggggatgacgtcaagtcatcatgccccttatgtccagggcttcacgcatgctacaatggctggtacagagagtggcgagcctgtgagggtgagcgaatctcggaaagccggtctcagttcggattggggtctgcaactcgacctcatgaagtcggagtcgctagtaatcgcagatcagcaacgctgcggtgaatacgttcccggggcttgtacacaccgcccgtcaagtcatgaaagttggtaacacccgaagccggtggcctaaccgttgtgggggagccgtcgaaggtgggactggtgattaggactaagtcgtaacaaggtagccgtaccggaaggtgcggctggatcacctcctttctaagga

SEQ ID NO: 11 - DNA, 1089 bp, synthetic construct
  misc_feature 1..1089: note = DNA sequence that encodes Dispersin B
  source 1..1089: mol_type = other DNA
atgaattgttgcgtaaaaggcaattccatatatccgcaaaaaacaagtaccaagcagaccggattaatgctggacatcgcccgacatttttattcacccgaggtgattaaatcctttattgataccatcagcctttccggcggtaattttctgcacctgcatttttccgaccatgaaaactatgcgatagaaagccatttacttaatcaacgtgcggaaaatgccgtgcagggcaaagacggtatttatattaatccttataccggaaagccattcttgagttatcggcaacttgacgatatcaaagcctatgctaaggcaaaaggcattgagttgattcccgaacttgacagcccgaatcacatgacggcgatctttaaactggtgcaaaaagacagaggggtcaagtaccttcaaggattaaaatcacgccaggtagatgatgaaattgatattactaatgctgacagtattacttttatgcaatctttaatgagtgaggttattgatatttttggcgacacgagtcagcattttcatattggtggcgatgaatttggttattctgtggaaagtaatcatgagtttattacgtatgccaataaactatcctactttttagagaaaaaagggttgaaaacccgaatgtggaatgacggattaattaaaaatacttttgagcaaatcaacccgaatattgaaattacttattggagctatgatggcgatacgcaggacaaaaatgaagctgccgagcgccgtgatatgcgggtcagtttgccggagttgctggcgaaaggctttactgtcctgaactataattcctattatctttacattgttccgaaagcttcaccaaccttctcgcaagatgccgcctttgccgccaaagatgttataaaaaattgggatcttggtgtttgggatggacgaaacaccaaaaaccgcgtacaaaatactcatgaaatagccggcgcagcattatcgatctggggagaagatgcaaaagcgctgaaagacgaaacaattcagaaaaacacgaaaagtttattggaagcggtgattcataagacgaatggggatgagtga

SEQ ID NO: 12 - protein, 362 aa, Aggregatibacter actinomycetemcomitans
MNCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 13 - DNA, 1103 bp, synthetic construct
  misc_feature 1..1103: note = DNA sequence that encodes an alginate lyase
  source 1..1103: mol_type = other DNA
atgaaaacgtcccacctgatccgtatcgccctgcccggtgccctcgccgcggcattgctcgccagccaggtcagccaggccgccgacctggtacccccgcccggctactacgcggcggtcggcgagcgcaagggcagcgccggcagctgccccgcggtgccgccgccgtataccggcagcctggtcttcaccagcaagtacgaaggctccgattcggcgcgggcgaccctcaacgtcaaggcggagaagaccttccgctcgcagatcaaggacatcaccgacatggagcgcggcgccaccaagctggtcacccagtacatgcgcagcggccgcgacggcgacctggcctgcgcactgaactggatgagcgcctgggcccgcgccggcgccctgcagagcgacgacttcaaccacaccggcaagtccatgcgcaaatgggcgctgggcagcctctccggcgcctacatgcgcctgaagttctccagctcgcggccgctcgcggcccacgccgagcagagccgggaaatcgaggactggttcgcccggctcggcacccaggtagtccgcgactggagcggcctgccgctgaagaagatcaacaaccattcctactgggcggcctggtcggtgatgtccaccgcggtggtgaccaaccgccgcgacctcttcgactgggcggtgagcgagttcaaggtcgccgccaaccaggtcgacgagcagggcttcctgcccaacgaactcaagcgccgccagcgcgccctcgcctaccacaactatgcgctgccaccgctggcgatgatcgccgcgttcgcccaggtcaacggcgtcgacctgcgccaggagaaccacggcgccctgcagcgcctggccgagcgggtgatgaagggagtcgacgacgaggaaaccttcgaggagaagaccggcgaggaccaggacatgaccgacctcaaggtcgacaacaagtacgcctggctggagccctactgcgccctctaccgctgcgagccgaagatgctcgaggcgaagaaggaccgcgagccgttcaacagtttccgcctcggcggcgaagtgacgcgggtgttcagccgcgaagggggaagttg

SEQ ID NO: 14 - protein, 367 aa, Flavobacterium multivorum
MKTSHLIRIALPGALAAALLASQVSQAADLVPPPGYYAAVGERKGSAGSCPAVPPPYTGSLVFTSKYEGSDSARATLNVKAEKTFRSQIKDITDMERGATKLVTQYMRSGRDGDLACALNWMSAWARAGALQSDDFNHTGKSMRKWALGSLSGAYMRLKFSSSRPLAAHAEQSREIEDWFARLGTQVVRDWSGLPLKKINNHSYWAAWSVMSTAVVTNRRDLFDWAVSEFKVAANQVDEQGFLPNELKRRQRALAYHNYALPPLAMIAAFAQVNGVDLRQENHGALQRLAERVMKGVDDEETFEEKTGEDQDMTDLKVDNKYAWLEPYCALYRCEPKMLEAKKDREPFNSFRLGGEVTRVFSREGGS

SEQ ID NO: 15 - DNA, 1539 bp, synthetic construct
  misc_feature 1..1539: note = DNA sequence that encodes an amylase
  source 1..1539: mol_type = other DNA
atgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacacctaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatag

SEQ ID NO: 16 - protein, 512 aa, Bacillus licheniformis
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 17 - DNA, 996 bp, synthetic construct
  misc_feature 1..996: note = DNA sequence that encodes a cellulase
  source 1..996: mol_type = other DNA
atgaagtttcagagcactttgcttcttgccgccgcggctggttccgcgttggctgtgcctcatggctccggacataagaagagggcgtctgtgtttgaatggttcggatcgaacgagtctggtgctgaatttgggaccaatatcccaggcgtctggggaaccgactacatcttccccgacccctcgaccatctctacgttgattggcaagggaatgaacttcttccgcgtccagttcatgatggagaggttgcttcctgactcgatgactggttcatacgacgaggagtatctggccaacttgacgactgtggtgaaagcggtcacggatggaggcgcgcatgcgctcatcgaccctcataactatggcagatacaacggggagatcatctccagtacatcggatttccagactttctggcagaatctggcgggccagtacaaagataacgacttggtcatgtttgataccaacaacgaatactacgacatggaccaggatctcgtgctgaatctcaaccaagcagccattaacggcatccgcgctgcaggtgcaagccagtacattttcgtcgaaggcaactcctggaccggagcttggacatgggtcgatgtcaacgataatatgaagaatttgaccgacccagaagacaagatcgtctatgaaatgcaccagtacctagactccgacggttccggcacttcggagacctgtgtctccgggacaatcggaaaggagcggatcactgatgctacacagtggctcaaggacaataagaaggtcggcttcatcggcgaatatgccggggggtccaatgatgtgtgtcggagtgccgtgtccgggatgctagagtacatggcgaacaacaccgacgtatggaagggtgcgtcgtggtgggcagccgggccatggtggggagactacattttcagcctggagcccccagatggaactgcttacacgggtatgctggatatcctggagacgtatctctga

SEQ ID NO: 18 - protein, 331 aa, Aspergillus niger
MKFQSTLLLAAAAGSALAVPHGSGHKKRASVFEWFGSNESGAEFGTNIPGVWGTDYIFPDPSTISTLIGKGMNFFRVQFMMERLLPDSMTGSYDEEYLANLTTVVKAVTDGGAHALIDPHNYGRYNGEIISSTSDFQTFWQNLAGQYKDNDLVMFDTNNEYYDMDQDLVLNLNQAAINGIRAAGASQYIFVEGNSWTGAWTWVDVNDNMKNLTDPEDKIVYEMHQYLDSDGSGTSETCVSGTIGKERITDATQWLKDNKKVGFIGEYAGGSNDVCRSAVSGMLEYMANNTDVWKGASWWAAGPWWGDYIFSLEPPDGTAYTGMLDILETYL

SEQ ID NO: 19 - DNA, 1155 bp, synthetic construct
  misc_feature 1..1155: note = DNA sequence that encodes proteinase K
  source 1..1155: mol_type = other DNA
atgcgtttgtctgttcttctgagtcttcttcccctcgctctcggcgctcctgccgttgagcagcgctccgaggctgctcctctgatcgaggcccgcggcgagatggttgccaacaagtacattgtcaagttcaaggagggtagcgctctttctgctctcgatgctgccatggagaagatttctggcaagcccgaccacgtctacaagaacgtcttcagtggtttcgctgcgacccttgacgagaacatggttcgggttctccgcgcccatcccgatgttgagtacattgagcaggatgctgttgtcaccatcaacgctgcgcagaccaacgctccctggggccttgctcgcatctccagcaccagccccggtacctctacttactactatgacgaatctgccggccaaggctcctgcgtctacgtgattgacaccggtatcgaggcatcgcaccccgagtttgagggtcgtgcccagatggtcaagacctactactactccagtcgcgacggtaacggtcacggcactcactgcgctggtaccgttggctcccgaacctacggtgtcgccaagaagacccagctctttggtgtcaaggtcctcgatgacaacggcagtggccagtactccaccatcatcgccggtatggactttgttgccagcgacaagaacaaccgcaactgccccaaaggtgtcgttgcctccttgtcccttggcggtggttactcctcctccgtgaacagcgccgctgccaggctccagagctctggtgtcatggtcgccgtcgctgccggtaacaacaacgctgacgcccgcaactactcccctgcttctgagccctcggtctgcactgtcggtgcttctgaccgctacgacagacgctccagcttctccaactacggcagcgttttggacatctttggccctggtaccagcattctctccacctggatcggcggcagcacccgctccatctctggaacttccatggctactccccacgttgccggtctcgctgcctacctcatgactcttggaaagactaccgccgccagcgcttgccgatacattgccgacaccgccaacaagggcgacttgagcaacattcccttcggcactgtcaacctgcttgcctacaacaactaccaggcttaa

SEQ ID NO: 20 - protein, 384 aa, Parengyodontium album
MRLSVLLSLLPLALGAPAVEQRSEAAPLIEARGEMVANKYIVKFKEGSALSALDAAMEKISGKPDHVYKNVFSGFAATLDENMVRVLRAHPDVEYIEQDAVVTINAAQTNAPWGLARISSTSPGTSTYYYDESAGQGSCVYVIDTGIEASHPEFEGRAQMVKTYYYSSRDGNGHGTHCAGTVGSRTYGVAKKTQLFGVKVLDDNGSGQYSTIIAGMDFVASDKNNRNCPKGVVASLSLGGGYSSSVNSAAARLQSSGVMVAVAAGNNNADARNYSPASEPSVCTVGASDRYDRRSSFSNYGSVLDIFGPGTSILSTWIGGSTRSISGTSMATPHVAGLAAYLMTLGKTTAASACRYIADTANKGDLSNIPFGTVNLLAYNNYQA

SEQ ID NO: 21 - DNA, 1140 bp, synthetic construct
  misc_feature 1..1140: note = DNA sequence that encodes subtilisin
  source 1..1140: mol_type = other DNA
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggaacttacagcggcattgtaagcggaatcgagtgggcgacgacaaacggcatggatgttatcaacatgagtcttggaggaccatcaggctcaacagcgatgaaacaggcggttgacaatgcatatgcaagaggggttgtcgttgtggcggctgctgggaacagcggatcttcaggaaacacgaatacaatcggctatcctgcgaaatacgactctgtcatcgcagttggcgcggtagactctaacagcaacagagcttcattttccagcgtcggagcagagcttgaagtcatggctcctggcgcaggcgtgtacagcacttacccaaccagcacttatgcaacattgaacggaacgtcaatggcttctcctcatgtagcgggagcagcagctttgatcttgtcaaaacatccgaacctttcagcttcacaagtccgcaaccgtctctccagtacggcgacttatttgggaagctccttctactatggaaaaggtctgatcaatgtcgaagctgccgctcaataa

SEQ ID NO: 22 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 23 - DNA, 669 bp, synthetic construct
  misc_feature 1..669: note = DNA sequence that encodes trypsin
  source 1..669: mol_type = other DNA
atcgtcgggggctacacctgcgcagagaattccgtcccttaccaggtgtccctgaatgctggctaccacttctgcgggggctccctcatcaatgaccagtgggtggtgtccgcggctcactgctaccagtaccacatccaggtgaggctgggagaatacaacattgatgtcttggagggtggtgagcagttcatcgatgcgtccaagatcatccgccaccccaagtacagcagctggactctggacaatgacatcctgctgatcaaactctccacgcctgcggtcatcaatgcccgggtgtccaccttgctgctgcccagtgcctgtgcttccgcaggcacagagtgcctcatctccggctggggcaacaccctgagcagtggcgtcaactacccggacctgctgcaatgcctggtggccccgctgctgagccacgccgactgtgaagcctcataccctggacagatcactaacaacatgatctgcgctggcttcctggaaggaggcaaggattcctgccagggtgactctggcggccctgtggcttgcaacggacagctccagggcattgtgtcctggggctacggctgtgcccagaagggcaagcctggggtctacaccaaggtctgcaactacgtggactggattcaggagaccatcgccgccaac

SEQ ID NO: 24 - protein, 223 aa, Bos taurus
IVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 25 - DNA, 1464 bp, synthetic construct
  misc_feature 1..1464: note = DNA sequence that encodes serratiopeptidase
  source 1..1464: mol_type = other DNA
atgcaatctactaaaaaggcaattgaaattactgaatccagcctcgctgccgcgacaaccggttacgatgctgtagacgacctgctgcattatcatgagcggggtaacgggattcagattaatggcaaggattcattttctaacgagcaagctgggctgtttattacccgtgagaaccaaacctggaacggttacaaggtatttggccagccggtcaaattaaccttctcgttcccggactataagttctcttccaccaacgtcgccggcgacaccgggctgagcaagttcagcgcggaacagcagcagcaggctaagctgtcgctgcagtcctgggccgacgtcgccaatatcaccttcaccgaagtggcggccggtcaaaaggccaatatcaccttcggcaactacagccaggatcgtcccggccactatgattacggcacccaggcctacgccttcctgccgaacaccatttggcagggccaggatttgggcggccagacttggtacaacgtaaaccaatccaacgtgaagcatccggcgaccgaagactacggccgccagacgttcacccatgagattggccatgcgctgggcctgagccacccgggcgactacaacgccggtgagggcaacccgacctatagagatgtcacctatgcggaagatacccgccagttcagcctgatgagctactggagtgaaaccaataccggtggcgacaacggcggtcactatgccgcggctccgctgctggatgacattgccgccattcagcatctgtatggcgccaacctgtcgacccgcaccggcgacaccgtgtacggctttaactccaataccggtcgtgacttcctcagcaccaccagcaactcgcagaaagtgatctttgcggcctgggatgcgggcggcaacgataccttcgacttctccggttacaccgctaaccagcgcatcaacctgaacgagaaatggttctccgacgtgggcggcctgaagggcaacgtgtcgatcgccgccggtgtgaccattgagaacgccattggcggttccggcaacgacgtgatcgtcggcaacgcggccaataacgtgctgaaaggcggcgcgggtaacgacgtgctgttcggcggcggcggggcggatgaattgtggggcggtgccggcaaagacatcttcgtgttctctgccgccagcgattccgcaccgggcgcttcagactggatccgcgacttccagaaagggatcgacaagatcgacctgtcgttcttcaataaagaagcgcagagcagcgatttcattcacttcgtcgatcacttcagcggcacggccggtgaggcgctgctgagctacaacgcgtccagcaacgtgaccgatttgtcggtgaacatcggtgggcatcaggcgccggacttcctggtgaaaatcgtcggccaggtagacgtcgccacggactttatcgtgtaa

SEQ ID NO: 26 - protein, 487 aa, Serratia sp.
MQSTKKAIEITESSLAAATTGYDAVDDLLHYHERGNGIQINGKDSFSNEQAGLFITRENQTWNGYKVFGQPVKLTFSFPDYKFSSTNVAGDTGLSKFSAEQQQQAKLSLQSWADVANITFTEVAAGQKANITFGNYSQDRPGHYDYGTQAYAFLPNTIWQGQDLGGQTWYNVNQSNVKHPATEDYGRQTFTHEIGHALGLSHPGDYNAGEGNPTYRDVTYAEDTRQFSLMSYWSETNTGGDNGGHYAAAPLLDDIAAIQHLYGANLSTRTGDTVYGFNSNTGRDFLSTTSNSQKVIFAAWDAGGNDTFDFSGYTANQRINLNEKWFSDVGGLKGNVSIAAGVTIENAIGGSGNDVIVGNAANNVLKGGAGNDVLFGGGGADELWGGAGKDIFVFSAASDSAPGASDWIRDFQKGIDKIDLSFFNKEAQSSDFIHFVDHFSGTAGEALLSYNASSNVTDLSVNIGGHQAPDFLVKIVGQVDVATDFIV

SEQ ID NO: 27 - DNA, 783 bp, synthetic construct
  misc_feature 1..783: note = DNA sequence that encodes a bovine pancreatic DNAse I
  source 1..783: mol_type = other DNA
ttgaagattgctgctttcaacattagaactttcggtgaaactaaaatgtctaacgctactttggcatcttacatcgttagaattgtcagaagatatgatatcgttttaattcaagaagttagagactctcacttggttgcagttggtaaattgttagactacttgaaccaagatgacccaaacacttaccactacgttgtttctgaaccattgggtagaaactcttacaaagaaagatacttattcttgttcagaccaaacaaagtttcagttttggatacttaccaatacgacgacggttgcgaatcttgtggtaacgattctttctccagagaacctgctgttgttaaattctcatcacactctaccaaggttaaagagttcgctatcgttgctttgcattctgctccttctgacgctgttgctgaaattaactctttgtacgacgtttacttagatgttcaacagaaatggcacttgaacgacgtcatgttgatgggtgactttaacgctgattgctcttatgttacttcttctcaatggtcttcaattagattgagaacatcttcaactttccaatggttaattcctgattccgctgataccactgctactagtaccaactgtgcttacgatagaatcgttgttgctggatcattattgcaatcttctgttgtcccaggttcagcggcccctttcgatttccaagctgcatatggtttgtctaatgaaatggctttagccatttctgatcactacccagttgaagtcacattgacataa

SEQ ID NO: 28 - protein, 260 aa, Bos taurus
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT